(19) United States
(12) Patent Application Publication
(10) Pub. No.: US 2024/0316170 A1
(43) Pub. Date: Sep. 26, 2024

(54) POLYPEPTIDES FOR CANCER TREATMENT

(71) Applicant: OXFORD VACMEDIX UK LIMITED, Oxford, Oxfordshire (GB)

(72) Inventor: Shisong JIANG, Oxford (GB)

(21) Appl. No.: 18/140,244

(22) PCT Filed: Oct. 27, 2021
(86) PCT No.: PCT/GB2021/052794

§ 371 Date: Apr. 27, 2023

(30) Foreign Application Priority Data
Oct. 28, 2020 (GB) 2017119.5

(51) Int. Cl. A61K 39/00; A61K 39/12; A61K 45/06

(52) U.S. Cl. CPC A61K 39/00115; A61K 39/12; A61K 2039/585; A61K 45/06; C12N 2710/20034

(57) ABSTRACT

The present invention relates to methods, polypeptides and compositions of said polypeptides and/or their encoding nucleic acids for the prophylactic vaccination and/or therapeutic treatment of cancer, and the use of polypeptides in treating and/or preventing cancer, and/or improving the therapeutic efficacy of agents for the treatment of cancer.

### CROSS-REFERENCE TO RELATED APPLICATIONS

This application is a continuation of International Patent Application No. PCT/GB2021/052794 filed Oct. 27, 2021, which application claims the benefit of United Kingdom Patent Application No. 2017119.5 filed on Oct. 28, 2020, both of which are incorporated herein by reference in their entireties.

### FIELD OF THE INVENTION

The invention provides methods, polypeptides and compositions of said polypeptides and/or their encoding nucleic acids for the prophylactic vaccination and/or therapeutic treatment of cancer, and the use of polypeptides in treating and/or preventing cancer, and/or improving the therapeutic efficacy of agents for the treatment of cancer.

### BACKGROUND OF THE INVENTION

Cancers may arise due to mutations in, introduction of, or ectopic expression of genes functionally linked to cell cycle regulation and which result in dysregulated cell division in a process called ‘transformation’. Such mutations may be loss-of-function mutations in tumour suppressor genes, leading to inability to inhibit progression through the cell cycle, and/or may be gain-of-function mutations in protooncogenes, resulting in ectopic signalling and acceleration through the cell cycle. For example, the viral HPV E6 and E7 oncoproteins, present in individuals having been infected with HPV, promote cell cycle progression via various mechanisms, amongst which inhibition of tumour suppressors p53 (E6) and pRB (E7).

A normal function of the immune system is the destruction of cancerous cells through recognition of certain tumour-specific antigens (‘TSAs’) and tumour-associated antigens (‘TAAs’), molecular markers, either cell surface or excreted, which distinguish healthy cells from abnormal cells. This is commonly referred to as the ‘eliminating phase’. Over time, due to ongoing mutations within diseased tissues, the pace of cancer growth may outstrip the ability of immune cells to destroy the cancerous cells. Amongst these mutations are those which allow the cancerous cells to evade detection by the immune system, or otherwise inhibit the function of the immune system. One example of such a mutation, and one which is common to a number of cancers, is the ectopic upregulation of survivin (also ‘BIRC5’), a member of the ‘inhibitor of apoptosis’ (‘IAP’) family. Intracellular expression of survivin can repress apoptosis of said cell. Once mutations accumulate to the extent that the rate of cancerous cell growth outmatches the ability of the immune system to destroy the cancer cells, the cancerous tissues continue to grow. This is known as the ‘escape phase’.

Cancer therapeutics are available in a wide range of modalities, including chemotherapy, surgery, and radiotherapy. Unfortunately, many existing treatment strategies offer little selectivity of diseased cells and/or tissues over healthy cells and/or tissues, resulting in a range of side effects during the course of treatment. For example, many chemotherapeutic agents focus on disrupting processes which are shared between normal healthy cells and cancer cells, thus affecting both indiscriminately. In one such example, cisplatin crosslinks DNA bases to prevent DNA repair and ultimately leads to apoptosis. This targets the DNA repair process in both normal and cancerous cells and thus damages healthy and diseased tissues alike. Many cancers can become resistant to certain therapeutic strategies over the course of therapeutic treatment, resulting in said strategies becoming ineffective for the treatment of the cancer.

Whilst these approaches have their place, immunotherapy is increasingly being explored as a more targeted therapeutic intervention. Immunotherapy co-opts the immunosurveillance of the elimination phase, artificially directing the immune system to particular molecular targets present on tumour cells. Immunotherapy aims to increase the ability of the subject's immune system to detect and destroy cancer cells.

There are currently many different immunotherapeutic strategies being employed against cancer, including immune checkpoint therapy, TNFR agonists, and targeted vaccination strategies. However, cancer vaccines remain limited in their applicability—often only to a small subset of patients—and efficacy. Similarly, checkpoint inhibitors and TNFR agonists likewise are limited in their efficacy by high toxicity profiles and limited efficacy.

What is required is a therapeutic approach which allows maximal efficacy of the abovementioned agents whilst minimizing the risk of toxic events over the course of treatment of a subject. Surprisingly, we have found that a combination of immuno-oncology agents and recombinant polypeptides derived from a target tumour antigen can improve the efficacy of immune therapy approaches in the treatment of cancer and also result in lower doses of the immuno-oncology agent being required, thus improving treatment outcomes and also reducing or eliminating the toxic effects of administration of said immuno-oncology agent.

### SUMMARY OF THE INVENTION

In a first aspect is provided a method for the treatment of cancer in a subject comprising: administering, to the subject, a polypeptide comprising two or more peptide fragments, wherein a first peptide fragment comprises a first sequence derived from a first tumour antigen protein and wherein a second peptide fragment comprises a second sequence derived from a second tumour antigen protein, further comprising one or more exogenous cathepsin cleavage site sequences located between each of the two or more peptide fragments; and administering, to the subject, an immuno-oncology agent.

In some embodiments the first tumour antigen protein and/or the second tumour antigen protein is a tumour specific antigen, a tumour associated antigen, or a cancer/testis antigen. In some embodiments, the first tumour antigen protein and second tumour antigen protein are the same tumour antigen protein. In some embodiments, the first tumour antigen protein and/or second tumour antigen protein is a self-antigen, an altered-self-antigen, or a non-self-antigen.

In some embodiments, the first tumour antigen protein and/or second tumour antigen protein is survivin. In other embodiments, the first tumour antigen protein and/or second tumour antigen protein is a viral-derived cancer antigen, optionally an HPV protein, further optionally an HPV16 protein. In some embodiments, the first tumour antigen protein and/or second tumour antigen protein is HPV16 E7.

In some embodiments, the one or more exogenous cathepsin cleavage site sequences is a cathepsin S cleavage sequence, preferably an LRMK cleavage sequence.

In some embodiments, the polypeptide and the immuno-oncology agent are administered to the subject simultaneously, separately, or sequentially. In some embodiments, the immuno-oncology agent is a TNFR Superfamily agonist, or a checkpoint inhibitor.

In some embodiments, each administration of the polypeptide comprises between 1 μg·kg⁻¹ to 2000 μg·kg⁻¹ of the polypeptide, preferably 5 to 20 μg·kg⁻¹ or lower.

In some embodiments, the TNFR Superfamily agonist is a peptide or fragment thereof, a glycoprotein or fragment thereof, or a small molecule. In some embodiments, the checkpoint inhibitor is a peptide or fragment thereof, a glycoprotein or fragment thereof, or a small molecule. In other embodiments, the TNFR Superfamily agonist is an antibody, or fragment thereof. In some embodiments, the checkpoint inhibitor is an antibody, or fragment thereof.

In some embodiments, the TNFR Superfamily agonist is administered at a dose non-toxic to humans.

In some embodiments, the TNFR Superfamily agonist is a 4-1BB agonist. In some embodiments, the checkpoint inhibitor is a PD-1 antagonist.

In some embodiments, the 4-1BB agonist is administered at a dose below 1 mg·kg⁻¹.

In some embodiments, the administration of the polypeptide and the immuno-oncology agent to the subject is repeated periodically, preferably every 3, 4, 5, 6, or 7 days.

In some embodiments, the two or more peptide fragments comprise one or more overlapping sequences. In some embodiments, the one or more overlapping sequences are between 2 and 31 amino acids in length. In some embodiments, the one or more overlapping sequences are at least 8 amino acids in length.

In some embodiments, the polypeptide is delivered in a delivery vehicle, optionally further comprising administering the delivery vehicle comprising the polypeptide or the polypeptide in a pharmaceutically acceptable carrier.

In a second aspect of the invention is provided a composition for use in the treatment of cancer, wherein the composition comprises a polypeptide comprising two or more peptide fragments, wherein a first peptide fragment comprises a first sequence derived from a first tumour antigen protein and wherein a second peptide fragment comprises a second sequence derived from a second tumour antigen protein, further comprising one or more exogenous cathepsin cleavage site sequences located between each of the two or more peptide fragments, and wherein the treatment comprises co-administration of the polypeptide with an immuno-oncology agent.

In some embodiments, the composition further comprising the polypeptide of any of the embodiments above or otherwise herein described.

In a third aspect of the invention is provided a method of determining whether a cancer is suitable for treatment according to the method of treatment above, comprising: administering to a subject or an in vitro sample a polypeptide comprising two or more peptide fragments, wherein a first peptide fragment comprises a first sequence derived from a first tumour antigen protein and wherein a second peptide fragment comprises a second sequence derived from a second tumour antigen protein, further comprising one or more exogenous cathepsin cleavage site sequences located between each of the two or more peptide fragments; administering to a subject or an in vitro sample an immuno-oncology agent; and measuring T cell stimulation in said subject or in vitro sample.

In a fourth aspect is provided an immuno-oncology agent for use in the treatment of cancer, wherein the treatment comprises administering the immuno-oncology agent and a polypeptide comprising two or more peptide fragments, wherein a first peptide fragment comprises a first sequence derived from a first tumour antigen protein and wherein a second peptide fragment comprises a second sequence derived from a second tumour antigen protein, further comprising one or more exogenous cathepsin cleavage site sequences located between each of the two or more peptide fragments.

In some embodiments, the polypeptide co-administered with said immuno-oncology agent for use in the treatment of cancer is a polypeptide as described above or in any of the embodiments herein. In some embodiments, the treatment of cancer is by the method of treatment as described above or in any of the embodiments herein.

In a fifth aspect is provided a kit for the treatment of cancer comprising: a polypeptide comprising two or more peptide fragments, wherein the first peptide fragment comprises a first sequence derived from a first tumour antigen protein and wherein the second peptide fragment comprises a second sequence derived from a second tumour antigen protein, further comprising one or more exogenous cathepsin cleavage site sequences located between each of the two or more peptide fragments, and an immuno-oncology agent.

In some embodiments, the immuno-oncology agent is a TNFR Superfamily agonist, optionally a peptide or fragment thereof, a glycoprotein or fragment thereof, a small molecule, or an antibody or fragment thereof. In some embodiments, the immuno-oncology agent is a checkpoint inhibitor, optionally a peptide or fragment thereof, a glycoprotein or fragment thereof, a small molecule, or an antibody or fragment thereof.

In some embodiments, the kit further comprises one or more pharmaceutically acceptable carriers or a nucleic acid encoding the polypeptide.

In some embodiments, the TNFR Superfamily agonist is a 4-1BB agonist, or wherein the checkpoint inhibitor is a PD-1 antagonist.

In some embodiments, the present invention provides a method for the treatment of cancer in a subject comprising administering, to the subject, a polypeptide comprising two or more peptide fragments, wherein a first peptide fragment comprises a first sequence derived from survivin and wherein a second peptide fragment comprises a second sequence derived from survivin, further comprising one or more protease cleavage site sequences located between each of the two or more peptide fragments, and administering, to the subject, an immuno-oncology agent.

In some embodiments, the polypeptide and the immuno-oncology agent are administered to the subject simultaneously, separately, or sequentially. In some embodiments, the immuno-oncology agent is a Tumour Necrosis Factor Receptor (TNFR) Superfamily agonist or a checkpoint inhibitor. In some embodiments, each administration of the polypeptide comprises between 1 μg·kg⁻¹ to 2000 μg·kg⁻¹ of the polypeptide, preferably 5 to 20 μg·kg⁻¹ or lower.

In some embodiments, the TNFR Superfamily agonist is a peptide or fragment thereof, a glycoprotein or fragment thereof, or a small molecule, In some embodiments, the checkpoint inhibitor is a peptide or fragment thereof, a glycoprotein or fragment thereof, or a small molecule. In some embodiments, the TNFR Superfamily agonist is an antibody or fragment thereof. In some embodiments, the checkpoint inhibitor is an antibody or fragment thereof. In some embodiments, the TNFR Superfamily agonist is administered at a dose non-toxic to humans.

In some embodiments, the TNFR Superfamily agonist is a 4-1BB agonist. In some embodiments, the checkpoint inhibitor is a PD-1 antagonist. In some embodiments, the 4-11BE agonist is administered at a dose below 1 mg·kg⁻¹.

In some embodiments, the administration of the polypeptide and the immuno-oncology agent to the subject is repeated periodically, preferably every 3, 4, 5, 6, or 7 days.

In some embodiments, the first and second peptide fragments each comprise a sequence with at least 90% identity to a contiguous sequence from SEQ ID NO: 1, and the polypeptide stimulates a T cell response in a subject, preferably a human subject. In some embodiments, the first and second peptide fragments each comprise a contiguous sequence from SEQ ID NO: 1.

In some embodiments, the first and second peptide fragments each comprise a sequence with at least 90% identity to a contiguous sequence from SEQ ID NO: 43, and the polypeptide stimulates a T cell response in a subject, preferably a human subject. In some embodiments, the first and second peptide fragments each comprise a contiguous sequence from SEQ ID NO: 43.

In some embodiments, the one or more protease cleavage site sequences is a cathepsin cleavage sequence, preferably cathepsin S, more preferably an LRMK cleavage sequence.

In some embodiments, the polypeptide comprises three or more peptide fragments, preferably five or more peptide fragments, more preferably ten or more peptide fragments.

In some embodiments, at least one of the two or more peptide fragments comprises a sequence with at least 90% identity to a sequence selected from the group: SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, and the polypeptide elicits an immune response or is immunostimulatory.

In some embodiments, the two or more peptide fragments comprise a sequence with at least 90% identity to SEQ ID NO: 11 and/or SEQ ID NO: 12, and the polypeptide elicits an immune response, optionally a T cell response.

In some embodiments, at least one of the two or more peptide fragments comprises a sequence with at least 90% identity to a sequence selected from the group: SEQ ID NO: 45, 46, 47, and 48, and the polypeptide elicits an immune response or is immunostimulatory.

In some embodiments, the two or more peptide fragments comprise a sequence with at least 90% identity to SEQ ID NO: 45, SEQ ID NO: 46, SEQ ID NO: 47, and/or SEQ ID NO: 48, and the polypeptide elicits an immune response, optionally a T cell response.

In some embodiments, the two or more peptide fragments comprise one or more overlapping sequences. In some embodiments, the one or more overlapping sequences are between 2 and 31 amino acids in length. Optionally, the one or more overlapping sequences are at least 8 amino acids in length.

In some embodiments, the polypeptide is delivered in a delivery vehicle.

In some embodiments, the method comprises administering the delivery vehicle comprising the polypeptide or the polypeptide in a pharmaceutically acceptable carrier.

In an embodiment, the invention provides a composition for use in a combination therapy for the treatment of cancer, wherein the composition comprises a polypeptide comprising two or more peptide fragments, wherein a first peptide fragment comprises a first sequence derived from survivin and wherein a second peptide fragment comprises a second sequence derived from survivin, further comprising one or more protease cleavage site sequences located between each of the two or more peptide fragments, and wherein the combination therapy comprises co-administration of the polypeptide with an immuno-oncology agent.

In some embodiments, the composition further comprises the polypeptide or method steps as described in any prior aspect or embodiment. In some embodiments, the first and second peptide fragments each comprise a sequence with at least 90% homology to a contiguous sequence from SEQ ID NO: 1, and the polypeptide stimulates a T cell response in a subject, preferably a human subject. In some embodiments, the first and second peptide fragments each comprise a contiguous sequence from SEQ ID NO: 1. In some embodiments, the one or more protease cleavage site sequences is a cathepsin cleavage sequence, preferably cathepsin S, more preferably an LRMK cleavage sequence. In some embodiments, the polypeptide comprises three or more peptide fragments, preferably five or more peptide fragments, more preferably ten or more peptide fragments.

In some embodiments, at least one of the two or more peptide fragments comprises a sequence with at least 90% homology to a sequence selected from the group: SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, and wherein the polypeptide elicits an immune response in the subject or is immunostimulatory.

In some embodiments, the two or more peptide fragments comprise a sequence with at least 90% homology to SEQ ID NO: 11 and/or SEQ ID NO: 12, and the polypeptide elicits an immune response in the subject, optionally a T cell response.

In some embodiments, the two or more peptide fragments comprise one or more overlapping sequences. In some embodiments, the one or more overlapping sequences are 2 and 31 amino acids in length. Optionally, the one or more overlapping sequences are at least 8 amino acids in length.

In some embodiments, the polypeptide is delivered in a delivery vehicle. In some embodiments, the composition comprises the delivery vehicle comprising the polypeptide or the polypeptide in a pharmaceutically acceptable carrier.

In some embodiments, the immuno-oncology agent is a TNFR Superfamily agonist or a checkpoint inhibitor. In some embodiments, the TNFR Superfamily agonist is a peptide or fragment thereof, a glycoprotein or fragment thereof, or a small molecule, or wherein the checkpoint inhibitor is a peptide or fragment thereof, a glycoprotein or fragment thereof, or a small molecule.

In some embodiments, the TNFR Superfamily agonist is an antibody or fragment thereof. In some embodiments, the TNFR Superfamily agonist is administered at a dose non-toxic to humans. In some embodiments, the TNFR Superfamily agonist is a 4-1BB agonist. In some embodiments, the 4-1BB agonist is administered at a dose below 1 mg·kg¹. In some embodiments, the checkpoint inhibitor is an antibody or fragment thereof. In some embodiments, the checkpoint inhibitor is a PD-1 antagonist.

In an embodiment, the present invention provides an immuno-oncology agent for use in a combination therapy for the treatment of cancer, wherein the combination treatment comprises administering the immuno-oncology agent and a polypeptide comprising two or more peptide fragments, wherein a first peptide fragment comprises a first sequence derived from survivin and wherein a second peptide fragment comprises a second sequence derived from survivin, further comprising one or more protease cleavage site sequences located between each of the two or more peptide fragments.

In some embodiments, the polypeptide is as described in any preceding aspect of the invention. In some embodiments, the first and second peptide fragments each comprise a sequence with at least 90% homology to a contiguous sequence from SEQ ID NO: 1, and the polypeptide elicits an immune response in a subject, optionally a T cell response.

In some embodiments, the first and second peptide fragments each comprise a contiguous sequence from SEQ ID NO: 1. In some embodiments, the one or more protease cleavage site sequences is a cathepsin cleavage sequence, preferably cathepsin S, more preferably an LRMK cleavage sequence. In some embodiments, the polypeptide comprises three or more peptide fragments, preferably five or more peptide fragments, more preferably ten or more peptide fragments.

In some embodiments, at least one of the two or more peptide fragments comprises a sequence with at least 90% homology to a sequence selected from the group: SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, and wherein the polypeptide elicits an immune response in the subject or is immunostimulatory.

In some embodiments, the two or more peptide fragments comprise a sequence with at least 90% homology to SEQ ID NO: 11 and/or SEQ ID NO: 12, and the polypeptide elicits an immune response in the subject, optionally a T cell response, preferably in a human subject.

In some embodiments, the two or more peptide fragments comprise one or more overlapping sequences. In some embodiments, the one or more overlapping sequences are between 2 and 31 amino acids in length. Optionally, the one or more overlapping sequences are at least 8 amino acids in length.

In some embodiments, the polypeptide is delivered in a delivery vehicle. In some embodiments, the composition comprises the delivery vehicle comprising the polypeptide or the polypeptide in a pharmaceutically acceptable carrier.

In some embodiments, the immuno-oncology agent is a TNFR Superfamily agonist or a checkpoint inhibitor. In some embodiments, the TNFR Superfamily agonist is a peptide or fragment thereof, a glycoprotein or fragment thereof, or a small molecule. In some embodiments, the checkpoint inhibitor is a peptide or fragment thereof, a glycoprotein or fragment thereof, or a small molecule.

In some embodiments, the TNFR Superfamily agonist is an antibody or fragment thereof. In some embodiments, the TNFR Superfamily agonist is administered at a dose non-toxic to humans. In some embodiments, the TNFR Superfamily agonist is a 4-1BB agonist. In some embodiments, the 4-1BB agonist is administered at a dose below 1 mg·kg⁻¹. In some embodiments, the checkpoint inhibitor is an antibody or fragment thereof. In some embodiments, the checkpoint inhibitor is a PD-1 antagonist.

In an embodiment, the invention provides a kit for the treatment of cancer comprising a polypeptide comprising two or more peptide fragments, wherein the first peptide fragment comprises a first sequence derived from survivin and wherein the second peptide fragment comprises a second sequence derived from survivin, further comprising one or more protease cleavage site sequences located between each of the two or more peptide fragments, and the kit further comprises an immuno-oncology agent.

In some embodiments, the immuno-oncology agent is a TNFR Superfamily agonist or a checkpoint inhibitor.

In some embodiments the TNFR Superfamily agonist is a peptide or fragment thereof, a glycoprotein or fragment thereof, or a small molecule. In some embodiments, the checkpoint inhibitor is a peptide or fragment thereof, a glycoprotein or fragment thereof, or a small molecule.

In some embodiments, the TNFR Superfamily agonist is an antibody or fragment thereof. In some embodiments, the checkpoint inhibitor is an antibody or fragment thereof.

In some embodiments, the kit further comprises one or more pharmaceutically acceptable carriers or a nucleic acid encoding the polypeptide.

In some embodiments, the TNFR Superfamily agonist is a 4-1BB agonist. In some embodiments the checkpoint inhibitor is a PD-1 antagonist.

### DESCRIPTION OF THE FIGURES

Aspects and embodiments of the invention will now be described with reference to the Figures and Examples in which:

FIG. 1. Images showing the expression of survivin taken at 40× magnification and using immunohistochemical (IHC) staining. The figures show haemotoxylin stained B16-F10 cells (A) and B16-GFP-survivin cells (B). Both samples were stained with anti-survivin mouse antibody followed by an HRP-conjugated goat anti mouse IgG, with a subsequent diaminobenzidine stain. Expression of survivin was indicated by the IHC staining of B16-GFP-survivin cells. The B16-F10 showed haemotoxylin (nuclei) staining only, demonstrating no survivin presence.

FIG. 2. A graph showing the tumour volume in mice treated over 23 days with (i) adjuvant (MPL) alone with Phosphate-Buffered Saline (PBS) as a control (black solid line), (ii) the polypeptide (‘ROP-Survivin’)+adjuvant (grey dashed line), (iii) a low dose (0.6 mg·kg⁻¹) of a 4-1BB agonist (BE0239)+adjuvant (black dashed line), and (iv) a combination of all three of the polypeptide+a low dose (0.6 mg·kg⁻¹) of a 4-1 BB agonist+adjuvant (black dotted line). At low doses of 4-1 BB agonist, the combination of all three of the polypeptide+4-1 BB agonist+adjuvant inhibits tumour volume growth to the largest extent, and to a greater extent than treatment with 4-1 BB agonist+adjuvant in the absence of polypeptide ROP-Survivin.

FIG. 3. A graph showing the survival rates of mice treated over 23 days with (i) adjuvant (MPL) with PBS as a control (large dotted line), (ii) S-polypeptide ROP-Survivin (‘ROP-Survivin’)+adjuvant (small dotted line), (iii) a low dose (0.6 mg·kg⁻¹) of a 4-1EE agonist+adjuvant (black dashed line), and (iv) a combination of all three of the polypeptide+a low dose (0.6 mg·kg⁻¹) of a 4-1EE agonist+adjuvant (solid black line). Mice receiving the combination of all three of the polypeptide+4-1 BB agonist+adjuvant had the highest survival rate; mice receiving only adjuvant had the lowest survival rate.

FIG. 4. A graph showing the tumour volume in mice treated over 23 days with (i) adjuvant (MPL) with PBS as a control (solid black line), (ii) S-polypeptide ROP-Survivin (‘ROP-Survivin’)+adjuvant (grey dashed line), (iii) a high dose (1.8 mg·kg⁻¹) of a 4-1BB agonist+adjuvant (black dashed line), and (iv) a combination of all three of the polypeptide+a high dose (1.8 mg·kg⁻¹) of a 4-1BB agonist+adjuvant (black dotted line).

FIG. 5. Electrophoretic gels demonstrating:

(a) successful synthesis of the S-polypeptide ROP-Survivin (with theoretical molecular weight 879 bp). Lane 1=DNA MW ladder; Lane 2=S-polypeptide ROP-Survivin PCR product;

(b) successful transformation and amplification of S-polypeptide ROP-Survivin-containing plasmids in E. coli, identified via Bam HI and Xho I cleavage. Lane 1=plasmid P1/YN8735; Lane 2=plasmid P2/YN8736; Lane 3=plasmid P3/YN8737; Lane 4=vector PYR1688 control.

FIG. 6. Plasmid map of engineered S-polypeptide ROP-Survivin expression plasmid ‘pYR1688’.

FIG. 7.

(a) Electrophoretic gel demonstrating, in lanes 1 and 3, the presence of S-polypeptide (ROP-Survivin, 879 bp) in plasmids extracted from pYR1688-transformed E. coli and digested with Nde I+Xho I. Lane 2 shows a plasmid not having the inserted S-polypeptide (ROP-Survivin).

(b) SDS-PAGE demonstrating successful induction of S-polypeptide ROP-Survivin (indicated by arrow). ‘0h’ lanes=Total protein before induction with IPTG; ‘3h’ lanes=Total protein induced by IPTG for 3 hours; ‘5h’ lanes=Total protein induced by IPTG for 5 hours; ‘BL21’ lane=null vector in host cell line (control).

FIG. 8. A graph showing the tumour volume in mice treated over 14 days with (i) adjuvant (MPL) with PBS as a control, (ii) mouse S-polypeptide ROP-Survivin (‘mROP-Survivin)+adjuvant, (iii) PD-1 antagonist, and (iv) a combination of all three of S-polypeptide ROP-Survivin+PD-1 antagonist+adjuvant, as indicated.

FIG. 9. A graph showing the body weight of mice treated over 14 days with (i) adjuvant (MPL) with PBS as a control, (ii) mouse S-polypeptide ROP-Survivin (‘mROP-Survivin)+adjuvant, (iii) PD-1 antagonist, and (iv) a combination of all three of S-polypeptide ROP-Survivin+PD-1 antagonist+adjuvant.

FIG. 10. ELISPOT analysis of IFN-γ release by activated splenocytes harvested from mice having received i) S-polypeptide ROP-Survivin+adjuvant, (ii) S-polypeptide ROP-Survivin+PD-1 antagonist+adjuvant, iii) PD-1 antagonist, iv) adjuvant (MPL), v) and vehicle (PBS), as indicated.

FIG. 11. Example vaccination regimen for therapeutic treatment of mice with ROP-HPV or Protein HPV16 E7. Day 0 marks day of inoculation with TC1 tumour cell line.

FIG. 12. A graph showing the TC-1 tumour volume in mice treated over 22 days with (i) ROP-HPV (‘ROP-HPV16E7’; hollow line), ii) Protein HPV16 E7 (dotted line), iii) Adjuvant (dash-dot line), and iv) vehicle (PBS; dashed line), as indicated.

FIG. 13. Survival curve for mice receiving i) ROP-HPV (‘ROP-HPV16E7’; square points), ii) Protein HPV16 E7 (circular points), iii) Adjuvant (triangle points), or iv) vehicle (PBS; diamond points), as indicated. Day 0=tumour inoculation.

FIG. 14. Example vaccination regimen for therapeutic combination treatment of mice with ROP-HPV (‘Vaccination’) and either 4-1BB agonistic antibody or anti-PD-1 antagonistic antibody (‘Antibody treatment’) Protein HPV16 E7. Day 0 marks day of inoculation with TC1 tumour cell line.

FIG. 15. A graph showing the TC-1 tumour volume in mice treated over 22 days with i) ROP-HPV (‘ROP-HPV16E7’; long-dashed hollow line), ii) ROP-HPV+4-1BB agonist (solid black line), iii) 4-1BB agonist (‘4-1BB’; short-dashed hollow line), iv) adjuvant (dashed-dotted line black line), v) vehicle (PBS; dashed black line).

FIG. 16. A graph showing the TC-1 tumour volume in mice treated over 47 days with i) ROP-HPV (‘ROP-HPV16E7’; long-dashed hollow line), ii) ROP-HPV+4-1BB agonist (solid black line), iii) 4-1BB agonist (‘a4-1 BB’; short-dashed hollow line), iv) adjuvant (dashed-dotted line black line), v) vehicle (PBS; dashed black line). Premature termination of data/lines is due to mouse death.

FIG. 17. Survival curve to 27 days for mice receiving i) ROP-HPV+4-1BB agonist (circle points), ii) ROP-HPV (square points), iii) adjuvant (upwards-triangle points), iv) 4-1 BB agonist (downwards-triangle points), or v) vehicle (PBS; diamond points). Day 0=tumour inoculation.

FIG. 18. Survival curve to 47 days for mice receiving i) ROP-HPV+4-1BB agonist, ii) ROP-HPV, iii) adjuvant, or iv) 4-1BB agonist, as indicated. Day 0=tumour inoculation.

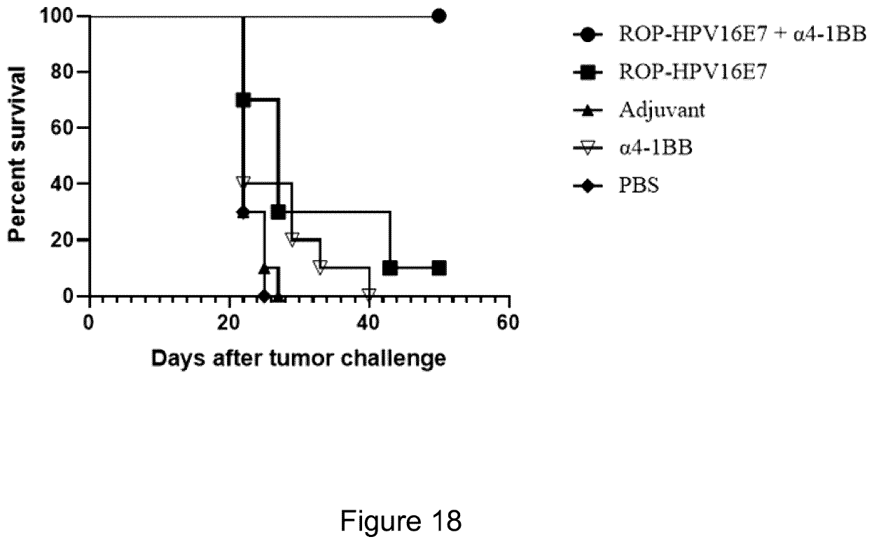

FIG. 19. A graph showing the tumour volume in mice treated over 47 days with i) ROP-HPV (‘ROP-HPV16E7’; hollow line), ii) ROP-HPV+anti-PD-1 antagonist (solid black line), iii) anti-PD-1 antagonist (‘αPD-1’; dotted line), iv) adjuvant (dashed-dotted line black line), or v) vehicle (PBS; dashed black line). Premature termination of data/lines is due to mouse death.

FIG. 20. Survival curve for mice receiving i) ROP-HPV (‘ROP-HPV16E7’), ii) ROP-HPV+anti-PD-1 antagonist, iii) anti-PD-1 antagonist, iv) adjuvant, or v) vehicle (PBS), as indicated. Day 0=tumour inoculation.

### DETAILED DESCRIPTION OF THE INVENTION

There are currently many different immunotherapeutic strategies being employed against cancer, including immune checkpoint therapy, adoptive T-cell transfer therapy, and vaccination (see Waldman et al., 2020). TSAs and TAAs are attractive vaccine candidates but require potent adjuvants to elicit effective responses, for example the 4-1 BBL trialled in Srivastava 2012 & Srivastava 2014. Molecule-targeted approaches are also common, aiming to specifically target molecular markers and/or molecular drivers of tumour progression. As noted in Wheatley and Altieri (2019), despite being a notable molecular marker of cancer, and despite possessing many desirable characteristics as a therapeutic target, “rather disappointingly, a truly specific anti-survivin agent is yet to reach the clinic.”

Two areas in immune therapy for cancer are the Tumor Necrosis Factor Receptor (‘TNFR’) Superfamily receptors (‘TNFRSF’ receptors), such as 4-1EE, and immune checkpoint molecules, such as PD-1 (Programmed cell death protein 1). 4-1BB is a co-stimulatory molecule which, when activated, causes T-cell expansion, cytokine induction, and upregulation of antiapoptotic genes. Therapeutic strategies typically focus on monoclonal antibody (mAb) agonists of 4-1BE, such as urelumab, which stimulate 4-1BB and thus produce potent anti-tumour effects. Unfortunately, 4-1BB agonism is associated with severe liver toxicity, and in mice have been shown to cause immune anomalies, affecting the function of the spleen, liver, and bone marrow (see Compte et al., 2018). These toxic profiles are described as the major hurdle for first generation 4-1 BB mAb agonists.

Likewise, PD-1 checkpoint inhibitors have been extensively explored as potential cancer-therapeutics, and numerous inhibitors of either PD-1, or it's ligand PD-L1, have been developed. PD-1 was first described in the early 90s. It negatively regulates T-cell-mediated immune responses. It is thought that activation of PD-1/PD-L1 may be one of the ways in which cancers evade antigen-specific T-cell responses, thus inhibition of this pathway may prevent cancers from mitigating the typical T-cell response against them. Numerous inhibitory antibody agents against PD-1 and PD-L1, such as Nivolumab and Atezolizumab, have been tested against a variety of cancers (See Gong et al., 2018). Much like with 4-1BB focused therapies, these checkpoint inhibitors often display non-trivial toxicity profiles affecting a variety of organs (see Spiers et al., 2019).

What is required is a therapeutic approach which allows maximal efficacy of the abovementioned agents whilst minimizing the risk of toxic events over the course of treatment of a subject.

To address this need, provided are polypeptides derived from tumour antigen proteins, for example tumour-specific antigens (TSAs), tumour-associated antigens (TAAs) or cancer/testis antigens, which, when co-administered with an immuno-oncology agent, exhibit anti-tumour activity. As shown herein, this anti-tumour activity can constitute tumour shrinkage and/or tumour regression, even complete regression. As shown herein, co-administration of a polypeptide of the invention with an immuno-oncology agent exhibits a synergistic effect.

Said polypeptides of the invention comprise two or more peptide fragments, wherein a first peptide fragment comprises a first sequence derived from a first tumour antigen protein, optionally a TSA or a TAA, and wherein a second peptide fragment comprises a second sequence derived from a second tumour antigen protein, optionally a TSA or a TAA. Said polypeptides comprise one or more exogenous protease cleavage site sequence(s) between each of the two or more peptide fragments, such that the polypeptide may be cleaved in vivo to liberate the one or more peptide fragments. The protease cleavage site sequence(s) is exogenous in that it is a protease cleavage site sequence which is not naturally present at its introduced location within the native tumour antigen protein sequence(s). In some embodiments said exogenous protease cleavage site sequence is a cathepsin cleavage sequence.

In one embodiment the polypeptide is derived from survivin which, when co-administered with a tumour necrosis family receptor (‘TNFR’) super family (‘TNFRSF’) agonist or a checkpoint inhibitor, may inhibit cancer growth at the maximum efficacy of the TNFRSF agonist or checkpoint inhibitor, whilst allowing each of the TNFRSF agonist or checkpoint inhibitor to be administered below its usual monotherapeutic dose. In another embodiment the polypeptide is derived from HPV16 E7 which, when co-administered with a tumour necrosis family receptor (‘TNFR’) super family (‘TNFRSF’) agonist or a checkpoint inhibitor, may inhibit cancer growth at the maximum efficacy of the TNFRSF agonist or checkpoint inhibitor, whilst allowing each of the TNFRSF agonist or checkpoint inhibitor to be administered below its usual monotherapeutic dose.

In other words, when combined with the polypeptide as disclosed herein, the TNFRSF agonist or checkpoint inhibitor may be administered at lower dosages (i.e. in smaller quantities) whilst still achieving a useful therapeutic effect. In some embodiments, the TNFRSF agonist or checkpoint inhibitor can be administered at lower dosages whilst still achieving their maximal therapeutic effect. The polypeptide derived from survivin comprises sequences derived from survivin linked to form an overlapping polypeptide which is capable of generating antibodies against cell surface proteins, and in addition stimulates CD4⁺ and CD8⁺ T cell responses. The polypeptide derived from survivin may be considered a variant thereof. The polypeptide derived from HPV16 E7 comprises sequences derived from HPV16 E7 linked to form an overlapping polypeptide which is capable of generating antibodies against cell surface proteins, and in addition stimulates CD4⁺ and CD8⁺ T cell responses. The polypeptide derived from HPV16 E7 may be considered a variant thereof.

The invention and terms used herein may be better understood with use of the following definitions.

“Co-administered” as used herein refers to two or more therapeutic or prophylactic substances which are both administered to a subject or patient as part of the same regimen. Co-administration may occur simultaneously, sequentially or separately in time. Co-administration may occur through the same route or through different routes. As an illustrative example, Substance 1 delivered intra-peritoneal every 3 days and Substance 2 delivered sub-cutaneous once per week could be said to be ‘co-administered’. As another illustrative example, Substance 1 and Substance 2 delivered sub-cutaneous on the same day could also be said to be ‘co-administered’.

“Recombinant” as used herein refers to any polymer, optionally a polypeptide, which is non-naturally occurring or artificially constructed, having been manufactured by gene recombination techniques in a bacterium (for example, but not limited to, an E. coli bacterium).

“Polypeptide” as used herein refers to a linear chain of amino acids linked by means of peptide bonds which is longer than a ‘peptide’ or ‘peptide fragment’, as used herein.

“Peptide” as used herein refers to a linear chain of amino acids linked by means of peptide bonds which is shorter than a ‘polypeptide’ as used herein.

“Amino acid sequence” as used herein means the identity of each amino acid residue in a peptide, polypeptide, or protein, including their order. This may be used interchangeably with ‘peptide sequence’.

“Peptide fragment” as used herein refers to an amino acid chain (a “peptide”) which is a piece of a larger polypeptide. In other words, two or more peptide fragments, if fragments of the same larger polypeptide, can together form all or part of the primary sequence of the larger polypeptide. In this case, the larger polypeptide is the recombinant polypeptide of the present invention.

“Protein” as used herein refers to a molecular entity composed primarily of one or more peptides and/or polypeptides and which has folded into, or presents as, a 3-dimensional conformation.

“Epitope” as used herein refers to a portion of a peptide fragment, peptide, polypeptide, protein, glycoprotein, lipoprotein, carbohydrate, lipid, or otherwise which is recognised by the adaptive immune system, preferably by T cells via their T Cell Receptor (‘TCR’).

“LRMK” as used herein refers to the Leu-Arg-Met-Lys amino acid sequence SEQ ID NO: 35, being a cleavage site recognised by interalia Cathepsin S. In some embodiments, a cleavable linker is provided and in some further embodiments, that linker is LRMK.

“Overlap” as used herein refers to a portion or ‘sub-sequence’ of an amino acid sequence which is the same, or substantially similar, in two different amino acid sequences, peptides, or peptide fragments preferably in such a way that the sub-sequence at the C-terminal end of one amino acid sequence, peptide or peptide fragment is the same as, or substantially similar to, the sub-sequence at the N-terminal end of another amino acid sequence, peptide, or peptide fragment and/or vice versa. Overlap may or may not be reflected in the polynucleotide sequences which encode said amino acid sequences.

“Identity” as used herein is the degree of similarity between two sequences, in other words the degree to which two sequences match one another in terms of residues, as determined by comparing two or more polypeptide or polynucleotide sequences. Identity can be determined using the degree of similarity of two sequences to provide a measurement of the extent to which the two sequences match. Numerous programs are well known by the skilled person for comparing polypeptide or polynucleotide sequences, for example (but not limited to) the various BLAST and CLUSTAL programs. Percentage identity can be used to quantify sequence identity. To calculate percentage identity, two sequences (polypeptide or nucleotide) are optimally aligned (i.e. positioned such that the two sequences have the highest number of identical residues at each corresponding position and therefore have the highest percentage identity) and the amino acid or nucleic acid residue at each position is compared with the corresponding amino acid or nucleic acid at that position. In some instances, optimal sequence alignment can be achieved by inserting space(s) in a sequence to best fit it to a second sequence. The number of identical amino acid residues or nucleotides provides the percentage identity, e.g. if 9 residues of a 10 residue long sequence are identical between the two sequences being compared then the percentage identity is 90%. Percentage identity is generally calculated along the full length of the two sequences being compared.

“Tumour antigen protein” as used herein refers to a protein produced in, on, or by a tumour cell and which (in the absence of immune suppression e.g. resulting from the tumour) stimulate immune response (i.e. is antigenic). ‘Tumour antigen proteins’ and ‘tumour antigens’ are used interchangeably herein. A tumour antigen protein may be a tumour associated antigen (TAA) or a tumour specific antigen (TSA), or a cancer/testis antigen.

“Variant” as used herein refers to a peptide, polypeptide, and/or protein which has an amino acid sequence with at least 60%, 65%, 70%, 75%, 80%, 85%, 90%, 91%, 92%, 93%, 94%, 95%, 96%, 97%, 98% or at least 99% identity, optionally 60-100%, 65-100%, 70-100%, 75-100%, 80-100%, 85-100%, 90-100%, 91-100%, 92-100%, 93-100%, 94-100%, 95-100%, 96-100%, 97-100%, 98-100% identity, to the wild-type peptide, polypeptide, and/or protein.

“Derived from” herein and throughout means ‘identical to or substantially similar to a portion of’. A protein fragment having a sequence derived from a survivin protein is a protein fragment containing an amino acid sequence which is identical to, or substantially similar to, a contiguous portion of the amino acid sequence of said survivin protein. ‘Substantially similar’ herein and throughout means that the amino acid sequence has at least 70%, 75%, 80%, 85%, 90%, 91%, 92%, 93%, 94%, 95%, 96%, 97%, 98% or at least 99% identity, optionally 70-100%, 75-100%, 80-100%, 85-100%, 90-100%, 91-100%, 92-100%, 93-100%, 94-100%, 95-100%, 96-100%, 97-100%, 98-100% identity to the reference wild-type Survivin protein sequence or a portion thereof. ‘At least’ herein and throughout means, in some embodiments, the recited percentage up to and including 100%. For example, ‘at least 75%’ can mean, in some embodiments, ‘75% to 100%’. Frequently, the nucleic acid sequence of a peptide fragment having a sequence derived from a survivin protein will differ from the survivin protein nucleic acid sequence to a greater degree than will the amino acid sequence of the peptide fragment from the survivin protein amino acid sequence. This is due to reasons of preparation and optimisation of expression of the polypeptide, for example codon optimisation. For the avoidance of doubt, it is the amino acid sequence of a peptide fragment which is derived from—in that it has at least 70%, 75%, 80%, 85%, 90%, 91%, 92%, 93%, 94%, 95%, 96%, 97%, 98% or at least 99% identity to a contiguous portion of—the amino acid sequence of a survivin protein. The nucleic acid sequences may differ to a greater extent and may have a lower sequence identity owing to the inherent redundancy of the genetic code for amino acids.

An “overlapping sequence” is a portion or sub-sequence of an amino acid sequence which is present in two or more peptide fragments of the polypeptide of the present invention. In some embodiments, the C-terminal end of one peptide fragment comprises an amino acid sequence which is the same as or substantially similar to the amino acid sequence at the N-terminal end of another peptide fragment. That means that where there is an overlapping sequence, there must be at least one portion of a peptide fragment which is the same on at least two peptide fragments. In some embodiments, the overlapping sequence is 2 to 40 amino acids in length, so each overlapping portion of a peptide fragment is 2 to 40 amino acids. In some embodiments, the overlapping sequence is 2 to 31 amino acids in length. In other embodiments, the overlapping sequence is 4 to 30 amino acids in length. In other embodiments, the overlapping sequence is 6 to 20 amino acids in length. In preferred embodiments, the overlapping sequence is 8 to 17 amino acids in length. In some embodiments, overlapping sequences are 8, 9, 10 or 11 amino acids in length. In some embodiments, overlapping sequences are 12 amino acids in length. In other embodiments, overlapping sequences are 13 amino acids, 14 amino acids, 15, 16, or 17 amino acids in length. In a most preferred embodiment, the overlapping sequence is at least 8 amino acids in length for the generation of a cytotoxic T lymphocyte (‘CTL’) (CD8⁺ T cell) response and/or at least 12 amino acids in length for the generation of a T helper cell (CD4⁺ T cell) response.

In one embodiment, the polypeptide of the invention comprises peptide fragments comprising a sequence which overlaps with that of one other peptide fragment within the polypeptide—for example, by means of its N-terminal sequence or its C-terminal sequence. In another embodiment, the polypeptide of the invention comprises peptide fragments comprising a sequence which overlaps with those of two other peptide fragments within the polypeptide—for example, by means of its N-terminal sequence and its C-terminal sequence. In some embodiments, the polypeptide of the invention additionally comprises one or more peptide fragment(s) which comprise a sequence which does not overlap with the sequence of any other peptide fragment contained within the polypeptide.

Any one peptide fragment may be 2 to 55 amino acids in length, more preferably 8 to 50 amino acids in length, more preferably 12 to 45 amino acids, more preferably 20 to 40 amino acids in length. In a preferred embodiment, each peptide fragment is 25 to 40 amino acids long, more preferably 28 to 38 amino acids long, even more preferably 29 to 37 amino acids long. In preferred embodiments, each peptide fragment is 29, 30, 31, 32, 33, 34, 35, 36, or 37 amino acids in length.

In all embodiments of the invention, peptide fragments are linked together in tandem to form the polypeptide by means of at least one protease cleavage site sequence located between each linearly adjacent peptide fragment. ‘Linearly adjacent’ is taken here to mean peptide fragments which are immediately sequential in terms of secondary structure or amino acid sequence. Accordingly, one or more protease cleavage site sequences separate each peptide fragment. Peptide fragments are connected by means of one or more protease cleavage site sequences. In one embodiment of the invention, two or more peptide fragments are linked together in tandem to form the polypeptide by means of at least one protease cleavage site sequence located between each linearly adjacent peptide fragment. In another embodiment, three or more peptide fragments are linked together in tandem to form the polypeptide by means of at least one protease cleavage site sequence located between each linearly adjacent peptide fragment. In another embodiment, 4 to 30, 5 to 20 peptide fragments, more preferably 10 to 15, 11 to 14, 12, or 13 peptide fragments are linked together in tandem to form the polypeptide by means of at least one protease cleavage site sequence located between each linearly adjacent peptide fragment.

The protease cleavage site is exogenous, meaning that it has been artificially introduced into the polypeptide and is not found in the wildtype sequence of the tumour antigen protein from which the polypeptide derives—at least in the location at which it has been introduced in the polypeptide sequence.

“Exogenous” as used herein means artificially introduced. It may also mean not present in the native sequence, for example the wild type (including any variants), at least in the location at which it is now artificially introduced. For example, a polypeptide may comprise two sequences which are contiguous in a native protein, and which are separated by an exogenous protease cleavage site i.e. a cleavage site which is not present in the contiguous native sequence. As another example, in the context of a polypeptide comprising peptide fragments comprising sequences derived from a tumour antigen protein and comprising an exogenous protease cleavage site between each peptide fragment, the exogenous protease cleavage site is a cleavage site that has been artificially introduced or which is not natively found in the tumour antigen protein at the location within the antigen protein amino acid sequence at which it is now located.

Where a dosage is expressed in ‘μg·kg⁻¹’, this is intended to mean the mass of the agent in micrograms per mass of the subject in kilograms. It will be clear to the skilled reader, therefore, that mg·kg⁻¹ means the mass of the agent in milligrams per mass of the subject in kilograms. The agent may be any of those listed herein i.e. the polypeptide, or the immuno-oncology agents. Said therapeutic and/or prophylactic polypeptide and/or an immuno-oncology agent may be provided to a mammalian subject, preferably a human.

In a first aspect, the present invention is a method for treatment of cancer in a subject comprising: administering, to the subject, a polypeptide comprising two or more peptide fragments, wherein a first peptide fragment comprises a first sequence derived from a first tumour antigen protein and wherein a second peptide fragment comprises a second sequence derived from a second tumour antigen protein, further comprising one or more exogenous cathepsin cleavage site sequences located between each of the two or more peptide fragments; and administering, to the subject, an immuno-oncology agent.

In a second aspect, the present invention is a composition for use in the treatment of cancer, wherein the composition comprises a polypeptide comprising two or more peptide fragments, wherein a first peptide fragment comprises a first sequence derived from a first tumour antigen protein and wherein a second peptide fragment comprises a second sequence derived from a second tumour antigen protein, further comprising one or more exogenous cathepsin cleavage site sequences located between each of the two or more peptide fragments, and wherein the treatment comprises co-administration of the polypeptide with an immuno-oncology agent.

The polypeptide of the present invention is particularly minded towards human tumour antigen proteins for the treatment of human cancers, although it will be readily apparent to the skilled person that the teachings are equally relevant to animal tumour antigen proteins for the treatment of cancers in animals. The tumour antigen protein may be a TAA, a TSA, or a cancer/testis antigen, all being proteins which are expressed exclusively or at elevated levels by tumour cells and therefore make attractive targets for vaccination.

TAAs suitable for use in a method or polypeptide of the invention will be readily apparent to the skilled person; they include, but are not limited to, Her2/neu, survivin, telomerase, BING-4, Cyclin-B1, 9D7, Ep-CAM, EphA3, mesothelin, SAP-1, Calcium-activated chloride channel 2. In one embodiment herein exemplified, the TAA is survivin. Survivin is a typical TAA; polypeptides and methods of the invention designed to target other TAAs will display similar efficacy, since it is feature common to all TAAs that they are self-antigens expressed at higher levels on tumour cells than on healthy cells.

TSAs suitable for use in a method or polypeptide of the invention will be readily apparent to the skilled person include. The TSA may be a development antigen ectopically expressed in the adult. The TSA may be a neoantigen and/or a mutated version of protein natively expressed by healthy cells. The TSA may be a viral-derived cancer antigen, being an antigen expressed by cancer cells with an oncogenic viral origin. Human oncogenic viruses include human papilloma virus (HPV), Epstein-Barr Virus (EBV), Hepatitis B virus (HBV), Hepatitis C Virus (HCV), Kaposi's sarcoma herpesvirus (KSHV), and Human T-cell lymphotropic virus-1 (HTLV-1).

In some embodiments, the viral-derived cancer antigen is HPV16 E7, HPV18 E7, HPV16 E6, HPV18 E6, EBV EBNA, EBV LMP-1, EBV LMP-2A, HBV HBx, HCV Core, HCV NS3, HCV Ns5A, HTLV-1 Tax, HTVL-1 HZB, KSHV vFLIP, KSHV LANA, KSHV vGPCR, KSHV vIRF-1. In one embodiment herein exemplified the TSA is HPV-derived cancer antigen E7. E7 proteins from HPV16 and HPV18 strains are particularly oncogenic. Herein exemplified is HPV16 E7. Polypeptides and methods of the invention designed to target other TSAs will display similar efficacy, since it is a feature common to all TSAs that they are expressed exclusively by tumour cells and not on healthy cells.

In some embodiments, the polypeptide of the invention comprises peptide fragments from only one tumour antigen protein. In some embodiments, the polypeptide of the invention comprises peptide fragments from more than one tumour antigen protein. As just one example, a polypeptide may comprise one or more peptide fragments comprising sequences derived from HPV16 E7, and also comprise one or more peptide fragments comprising sequences derived from HPV16 E6. Said HPV16 E6 and E7 peptide fragments may each be separated by an exogenous protease cleavage site sequence.

The present invention is a method for the treatment of cancer in a subject comprising administering a polypeptide comprising two or more peptide fragments, wherein the first peptide fragment comprises a first sequence derived from a first tumour antigen protein and wherein the second peptide fragment comprises a second sequence derived from a second tumour antigen protein, further comprising one or more protease cleavage site sequences located between each of the two or more peptide fragments and wherein said method further comprises administering, to the subject, an immuno-oncology agent. In some embodiments, the subject is a mammal. In some embodiments, the subject is a human. In some embodiments, the polypeptide comprises two or more peptide fragments, wherein the first peptide fragment consists of a first sequence derived from a first tumour antigen protein and wherein the second peptide fragment consists of a second sequence derives from a second tumour antigen protein, further comprising one or more protease cleavage site sequences located between each of the two or more peptide fragments.

The first sequence, second sequence, and any further sequences may be survivin sequences. The first sequence, second sequence, and any further sequences may be variants of all or part of the survivin sequences as outlined above.

The first sequence, second sequence, and any further sequences may be HPV16 E7 sequences. The first sequence, second sequence, and any further sequences may be variants of all or part of the HPV16 E7 sequences as outlined above.

Administration of the polypeptide and/or immuno-oncology agent (collectively, ‘the agents’) can be in a variety of ways and each agent can be delivered in the same way or in different ways. In some embodiments, one or more of the agents is administered by oral delivery, nasal spray, or injection. In some embodiments, delivery by injection can be by subcutaneous, intravenous, intramuscular, intraperitoneal, or intradermal injection. Administration of one or more of the agents can be simultaneous meaning in a single administration, separate meaning at least two of the agents are administered separately but at the same or different times, or sequentially meaning that none of the agents are administered together. In some embodiments, separately may mean that two or more agents are delivered within 1 minute, 2 minutes, 3 minutes, 4 minutes or 5 minutes, or more, of each other. In some embodiments, sequentially may mean on the same day, or on different days.

The polypeptide comprises at least two or more peptide fragments. In some embodiments it may comprise three or more peptide fragments, four or more peptide fragments, five or more peptide fragments, six or more peptide fragments, seven or more peptide fragments, eight or more peptide fragments, nine or more peptide fragments, ten or more peptide fragments, eleven or more peptide fragments, or twelve or more peptide fragments. In some embodiments it may comprise more than twelve peptide fragments.

The polypeptide has at least two peptide fragments having peptide sequences which are derived from or variants of one or more tumour antigen protein(s). The polypeptide may comprise two or more peptide fragments having the same sequence. Repeating certain peptide fragments in this way can be conformationally advantageous. The polypeptide may comprise two or more peptide fragments having a substantially similar sequence, in that the peptide fragment sequences have at least 80% sequence identity, optionally at least 90% sequence identity, to each other and/or in that the peptide fragment sequences differ by up to 4, 3, 2, or 1 amino acids. This can allow for various cancer-associated single-nucleotide polymorphisms to be represented within the polypeptide. Equally, the polypeptide may comprise two or more peptide fragments having different sequences, in that the sequences are derived from distinct and non-overlapping portions of one or more tumour antigen protein(s). This can allow for different, optionally epitopic, sequences within a given tumour antigen protein to be represented, or for different sequences deriving from two or more tumour antigen proteins associated with a tumour to be represented within the polypeptide. In some embodiments, the polypeptide may comprise two or more peptide fragments having sequences which overlap. This can allow for full and even multiple coverage of one or more epitopes within a given tumour antigen protein, with the advantage that broad T cell responses can be elicited in a population in a HLA-type independent manner. Equally, the polypeptide may comprise multiple peptide fragments, some of which having the same sequence, some of which having substantially similar sequences, some of which having different sequences, some of which having overlapping sequences, or any combination thereof.

In some embodiments, the polypeptide has at least two peptide fragments having peptide sequences which are derived from or variants of survivin, also known as BIRC5 (Baculoviral IAP repeat-containing protein 5). An exemplary protein sequence of survivin is included herein as SEQ ID NO: 1.

(142 aa)
SEQ ID NO: 1
MGAPTLPPAW QPFLKDHRIS TFKNWPFLEG CACTPERMAE
AGFIHCPTEN EPDLAQCFFC FKELEGWEPD DDPIEEHKKH
SSGCAFLSVK KQFEELTLGE FLKLDRERAK NKIAKETNNK
KKEFEETAKK VRRAIEQLAA MD

This relates to survivin isoform 1 (uniprot identifier O15392-1), but in some embodiments the sequences could be derived from or variants of one or more of survivin isoform 2 (uniprot identifier O15392-2, SEQ ID NO: 2), 3 (uniprot identifier O15392-3, SEQ ID NO: 3), 4 (uniprot identifier O15392-4, SEQ ID NO: 4), 5 (uniprot identifier O15392-5, SEQ ID NO: 5), 6 (uniprot identifier O15392-6, SEQ ID NO: 6), or 7 (uniprot identifier O15392-7, SEQ ID NO: 7).

(165 aa)
SEQ ID NO: 2
MGAPTLPPAW QPFLKDHRIS TFKNWPFLEG CACTPERMAE
AGFIHCPTEN EPDLAQCFFC FKELEGWEPD DDPIGPGTVA
YACNTSTLGG RGGRITREEH KKHSSGCAFL SVKKQFEELT
LGEFLKLDRE RAKNKIAKET NNKKKEFEET AKKVRRAIEQ
LAAMD
(137 aa)
SEQ ID NO: 3
MGAPTLPPAW QPFLKDHRIS TFKNWPFLEG CACTPERMAE
AGFIHCPTEN EPDLAQCFFC FKELEGWEPD DDPMQRKPTI
RRKNLRKLRR KCAVPSSSWL PWIEASGRSC LVPEWLHHFQ
GLFPGATSLP VGPLAMS
(120 aa)
SEQ ID NO: 4
MGAPTLPPAW QPFLKDHRIS TFKNWPFLEG CACTPERMAE
AGFIHCPTEN EPDLAQCFFC FKELEGWEPD DDPIEEHKKH
SSGCAFLSVK KQFEELTLGE FLKLDRERAK NKIERALLAE
(117 aa)
SEQ ID NO: 5
MGAPTLPPAW QPFLKDHRIS TFKNWPFLEG CACTPERMAE
AGFIHCPTEN EPDLAQCFFC FKELEGWEPD DDPIEEHKKH
SSGCAFLSVK KQFEELTLGE FLKLVRETLP PPRSFIR
(78 aa)
SEQ ID NO: 6
MGAPTLPPAW QPFLKDHRIS TFKNWPFLEG CACTPERMAE
AGFIHCPTEN EPDLAQCFFC FKELEGWEPD DDPMRELC
(74 aa)
SEQ ID NO: 7
MGAPTLPPAW QPFLKDHRIS TFKNWPFLEG CACTPERMAE
AGFIHCPTEN EPDLAQCFFC FKELEGWEPD DDPM

The skilled reader will understand that a nucleic acid sequence (DNA or RNA, or a mix of both) can be provided for each of the above-mentioned peptides, and that this would be routine for the person skilled in the art to derive. For example, the DNA sequence encoding SEQ ID NO: 1 is given below.

(426 bp)
SEQ ID NO: 20
ATGGGCGCCCCCACCCTGCCCCCCGCCTGGCAGCCCTTCCTGAAGGACC
ACAGGATCAGCACCTTCAAGAACTGGCCCTTCCTGGAGGGCTGCGCCTG
CACCCCCGAGAGGATGGCCGAGGCCGGCTTCATCCACTGCCCCACCGAG
AACGAGCCCGACCTGGCCCAGTGCTTCTTCTGCTTCAAGGAGCTGGAGG
GCTGGGAGCCCGACGACGACCCCATCGAGGAGCACAAGAAGCACAGCAG
CGGCTGCGCCTTCCTGAGCGTGAAGAAGCAGTTCGAGGAGCTGACCCTG
GGCGAGTTCCTGAAGCTGGACAGGGAGAGGGCCAAGAACAAGATCGCCA
AGGAGACCAACAACAAGAAGAAGGAGTTCGAGGAGACCGCCAAGAAGGT
GAGGAGGGCCATCGAGCAGCTGGCCGCCATGGAC

The first peptide fragment derived from survivin and the second peptide fragment derived from survivin can be from the same isoform, or from different isoforms, or variants thereof. Any of the peptide fragments of the polypeptide can comprise an amino-acid sequence from any of the isoforms listed above. In some embodiments the first peptide fragment and the second peptide fragment comprise a first sequence and a second sequence, respectively, wherein the sequences are from contiguous sections of the survivin sequence. As an illustrative example, the first sequence may comprise residues 1 to 10 of the protein from which the sequence is derived (e.g. survivin isoform 1), and the second sequence may comprise residues 11 to 20 of said protein sequence, and so on. Any residue from any of the above-mentioned isoforms may form the starting point for the first sequence or second sequence derived from survivin or variants thereof.

It will be understood that in cases where there are three or more peptide fragments, each of these will have an amino acid sequence which is a variant of or derived from survivin. The sequence may be identical between peptide fragments or may be different between each peptide fragment. As an illustrative example, the first peptide fragment may have a first comprising residues 1 to 10 from e.g. survivin isoform 1, the second peptide fragment may have a second sequence comprising residues 11 to 20, and the third peptide fragment may have the first sequence comprising residues 11 to 20.

In some embodiments, any of the two or more peptide fragments may comprise an ‘overlapping sequence’. An overlapping sequence means that a portion or sub-sequence of an amino acid sequence are the same, or substantially similar, in each of the two peptide fragments of the polypeptide. As an illustrative example, the first peptide fragment may comprise residues 1 to 10 of the amino acid sequence of the protein from which the fragment is derived (e.g. survivin isoform 1), and the second peptide fragment may comprise residues 5 to 15 of said protein sequence, thus the residues in each peptide fragment corresponding to residues 5 to 10 of the protein sequence each peptide fragment is derived from are the ‘overlapping sequence’. Polypeptides comprising these overlapping sequences may be referred to as recombinant overlapping polypeptides (ROPs). ROPs have been shown to provide advantages over conventional vaccines (see Cai et al., 2017, WO2007125371, and WO2016095812).

In some embodiments, the polypeptide has at least two peptide fragments having peptide sequences which are derived from or variants of HPV E7. An exemplary protein sequence of HPV E7 is included herein as SEQ ID NO: 43.

SEQ ID NO: 43 (98 aa):
MHGDTPTLHEYMLDLQPETTDLYCYEQLNDSSEEEDEIDGPAGQAEPDR
AHYNIVTFCCKCDSTLRLCVQSTHVDIRTLEDLLMGTLGIVCPICSQKP

This relates to wildtype HPV16 E7. However, in some embodiments, the protein sequence of HPV E7 may be HPV18 E7 (SEQ ID NO: 44), or the E7 protein of any HPV variant.

SEQ ID NO: 44 (105 aa):
MHGPKATLQDIVLHLEPQNEIPVDLLCHEQLSDSEEENDEIDGVN
HQHLPARRAEPQRHTMLCMCCKCEARIKLVVESSADDLRAFQQLF
LNTLSFVCPWCASQQ

The skilled reader will understand that a nucleic acid sequence (DNA or RNA, or a mix of both) can be provided for each of the above-mentioned peptides, and that this would be routine for the person skilled in the art to derive. For example, a DNA sequence encoding SEQ ID NO: 43 is given below.

(294 bp)
SEQ ID NO: 52
ATGCACGGCGACACCCCCACCCTGCACGAGTACATGCTGGACCTG
CAGCCCGAGACCACCGACCTGTACTGCTACGAGCAGCTGAACGAC
AGCAGCGAGGAGGAGGACGAGATCGACGGCCCCGCCGGCCAGGCC
GAGCCCGACAGGGCCCACTACAACATCGTGACCTTCTGCTGCAAG
TGCGACAGCACCCTGAGGCTGTGCGTGCAGAGCACCCACGTGGAC
ATCAGGACCCTGGAGGACCTGCTGATGGGCACCCTGGGCATCGTG
TGCCCCATCTGCAGCCAGAAGCCC

A codon-optimized sequence version of the above sequence may be used in place of the above SEQ ID NO: 52.

The first peptide fragment derived from HPV E7 and the second peptide fragment derived from HPV E7 can be from the same viral variant, or from different viral variant, or be sequence variants thereof. In some embodiments the first peptide fragment and the second peptide fragment comprise a first sequence and a second sequence, respectively, wherein the sequences are from contiguous sections of the HPV16 E7 sequence. As an illustrative example, the first sequence may comprise residues 1 to 10 of the protein from which the sequence is derived (e.g. HPV16 E7), and the second sequence may comprise residues 11 to 20 of said protein sequence, and so on. Any residue from any of the above-mentioned isoforms may form the starting point for the first sequence or second sequence derived from HPV16 E7 or variants thereof.

It will be understood that in cases where there are three or more peptide fragments, each of these will have an amino acid sequence which is a variant of or derived from HPV E7. The sequence may be identical between peptide fragments or may be different between each peptide fragment. As an illustrative example, the first peptide fragment may have a first comprising residues 1 to 10 from e.g. HPV16 E7, the second peptide fragment may have a second sequence comprising residues 11 to 20, and the third peptide fragment may have the first sequence comprising residues 11 to 20.

In some embodiments, any of the two or more peptide fragments may comprise an ‘overlapping sequence’. An overlapping sequence means that a portion or sub-sequence of an amino acid sequence are the same, or substantially similar, in each of the two peptide fragments of the polypeptide. As an illustrative example, the first peptide fragment may comprise residues 1 to 10 of the amino acid sequence of the protein from which the fragment is derived (e.g. HPV16 E7), and the second peptide fragment may comprise residues 5 to 15 of said protein sequence, thus the residues in each peptide fragment corresponding to residues 5 to 10 of the protein sequence each peptide fragment is derived from are the ‘overlapping sequence’. Polypeptides comprising these overlapping sequences may be referred to as recombinant overlapping polypeptides (ROPs). ROPs have been shown to provide advantages over conventional vaccines (see Cai et al., 2017, WO2007125371, and WO2016095812).

In some embodiments, the polypeptide may comprise multiple overlapping sequences. As an illustrative example, the first peptide fragment may comprise residues 1 to 10, the second peptide fragment may comprise residues 5 to 15, and the third peptide fragment may comprise residues 11 to 20. Thus, in the illustrative example, there are two overlapping sequences in the polypeptide, specifically residues 5 to 10 in the first and second peptide fragments, and 11 to 15 in the second and third peptide fragments. In addition, or alternatively, there may be one or more overlapping sequences, but not all of the peptide fragments need contain an overlapping sequence. As an illustrative example, the first and second peptide fragments may contain an overlapping sequence defined by residues 5 to 10, but the third peptide fragment may comprise residues 16 to 25, and thus not overlap with either. In some embodiments the polypeptide may comprise 2, 3, 4, 5, 6, 7, 8, 9, 10, 11, 12, 13, 14, 15, 16, 17, 18, 19, 20, 21, 22, 23, 24, 25 or more overlapping sequences.

In some embodiments, none of the two or more peptide fragments overlap. In some embodiments, the polypeptide comprises no overlapping sequence.

Any number of overlaps may be present and this is only limited by the number and size of the peptide fragments of the polypeptide.

In some embodiments, the polypeptide comprises a sequence which covers the whole amino acid sequence of a tumour antigen protein of a cancer of interest. In some embodiments, the polypeptide comprises a sequence which covers the whole amino acid sequence of a portion of interest of a tumour antigen protein of a cancer of interest. For example, the portion of interest may be the portion of the tumour antigen protein which is immunogenic and/or which comprises most or all T cell epitope regions and/or antibody epitopes; as another example the portion of interest may be the portion of the tumour antigen protein which is exposed extracellularly. In some embodiments, the polypeptide comprises a sequence representing at least 10% of the tumour antigen protein or the portion of interest, at least 20%, 30%, 40%, 50% of the tumour antigen protein or the portion of interest, preferably at least 60%, at least 70%, at least 80% or at least 90% of the tumour antigen protein or the portion of interest. In some embodiments, the polypeptide comprises a sequence representing 100% of the sequence of a tumour antigen protein or the portion of interest.

In some embodiments, the polypeptide may comprise a peptide fragment with a sequence having partial sequence identity to the wild-type tumour antigen protein sequence (e.g. survivin including any of the isoforms listed above, or their homologues, or e.g. HPV E7 including any of the viral variant E7 sequences). As an illustrative example, at least one peptide fragment may comprise a sequence with at least 99% identity to the relevant part of the tumour antigen protein sequence. Alternatively, at least one peptide fragment may comprise a sequence with at least 98%, 97%, 96%, 95%, 94%, 93%, 92%, 91%, 90%, 80%, 70%, 60%, 50%, 40%, 30%, 20%, or 10% identity to the relevant part of the tumour antigen protein sequence. ‘Relevant part’ means the contiguous string of residues of the tumour antigen protein sequence on which the peptide fragment in question is based. As an illustrative example, if the peptide fragment comprises a sequence with at least 90% identity to residues 1 to 10 of the tumour antigen protein (including by way of example survivin isoform 1), then 9 of the 10 residues will be identical to residues 1 to 10 of tumour antigen protein (again, by way of example survivin), and one will be different. The skilled reader will understand that any residues can be interchanged provided the percentage identity is intact. The skilled reader will further understand that a lower percentage identity is acceptable provided key residues are maintained.

Each of the two or more peptide fragments can be any length in terms of amino acids. Each of the two or more peptide fragments could be 1, 2, 3, 4, 5, 6, 7, 8, 9, 10, 11, 12, 13, 14, 15, 16, 17, 18, 19, 20, 21, 22, 23, 24, 25, 26 27, 28, 29, 30, 31, 32, 33, 34, 35 or more amino acids in length. The overlap between peptide fragments (i.e. the overlapping sequences) may be limited by the length of the peptide fragment, and these overlapping sequences may be at least 2, 3, 4, 5, 6, 7, 8, 9, 10, 11, 12, 13, 14, 15 or more amino acids in length. In some embodiments, the overlap is between 2 and 31 amino acids in length, optionally at least 8 amino acids in length.

The two or more peptide fragments of the polypeptide may comprise one or more sequences which cover the whole sequence of the protein. As an illustrative example, the polypeptide may comprise two peptide fragments, the first peptide fragment having a sequence derived from residues 1 to 71 of survivin isoform 1, the second fragment having a sequence derived from residues 72 to 142 of survivin isoform 1. The skilled reader will understand that any number of fragments may be used to cover the whole sequence of the survivin isoform upon which the polypeptide is based. As a further illustrative example, the polypeptide may comprise three polypeptide fragments, the first peptide fragment having a first sequence derived from residues 1 to 71 of survivin isoform 1, the second peptide fragment having a second sequence derived from residues 72 to 142 of survivin isoform 1, and the third peptide fragment having a third sequence derived from residues 50 to 120 of survivin isoform 1.

Such a polypeptide may comprise any number of overlapping sequences, it can comprise peptide fragments of any length, and the polypeptide sequence can be any length provided the peptide fragments are derived from tumour antigen protein (including, by way of example, survivin or variants thereof or HPV16 E7 or variants thereof as outlined above).

In some embodiments, the polypeptide of the invention is immunostimulatory. In some embodiments, one or more of the peptide fragments of the polypeptide of the invention are immunostimulatory. In some embodiments, one or more of the sequences comprised within the peptide fragments of the polypeptide of the invention are immunostimulatory. ‘Immunostimulatory’ as referred to herein means stimulates, motivates, causes, and/or produces an immune response when administered to a subject. In preferred embodiments, said immune response comprises an adaptive immune response. In some embodiments, said adaptive immune response comprises the generation of antibodies against the polypeptide and/or against one or more peptide fragments and/or sequences comprised therein. In other embodiments, said adaptive immune response comprises the activation and/or proliferation of CD8+ and/or CD4+ T cells. In some embodiments, said adaptive immune response comprises the generation of antibodies against the polypeptide and/or against one or more peptide fragments and/or sequences comprised therein and, further, the activation and/or proliferation of CD8+ and/or CD4+ T cells.

In one embodiment, the polypeptide comprises two or more peptide fragments each comprising a sequence derived from survivin, or a variant thereof, wherein the polypeptide stimulates a T cell response. In some embodiments, said two or more peptide fragments each comprising a sequence derived from survivin, or a variant thereof, comprise one or more of the following sequences:

(30 aa)
SEQ ID NO: 8
MGAPTLPPAWQPFLKDHRISTFKNWPFLEG
(30 aa)
SEQ ID NO: 9
DHRISTFKNWPFLEGCACTPERMAEAGFIH
(28 aa)
SEQ ID NO: 10
ACTPERMAEAGFIHCPTENEPDLAQCFF
(29 aa)
SEQ ID NO: 11
PTENEPDLAQCFFCFKELEGWEPDDDPIE
(30 aa)
SEQ ID NO: 12
FKELEGWEPDDDPIEEHKKHSSGCAFLSVK
(28 aa)
SEQ ID NO: 13
EHKKHSSGCAFLSVKKQFEELTLGEFLK
(29 aa)
SEQ ID NO: 14
QFEELTLGEFLKLDRERAKNKIAKETNNK
(30 aa)
SEQ ID NO: 15
RERAKNKIAKETNNKKKEFEETAEKVRRAI
(21 aa)
SEQ ID NO: 16
KEFEETAEKVRRAIEQLAAMD

optionally wherein the polypeptide is capable of eliciting an immune response or is immunostimulatory as defined above.

In some embodiments, the polypeptide comprises two or more peptide fragments each consisting of a sequence derived from survivin or a variant thereof, wherein the polypeptide stimulates a T cell response. In some embodiments said two or more peptide fragments consist of one or more of the sequences SEQ ID NOs: 8 to 16.

In one embodiment, the polypeptide comprises two or more peptide fragments each comprising a sequence derived from HPV16 E7, or a variant thereof, wherein the polypeptide stimulates a T cell response. In some embodiments, said two or more peptide fragments each comprising a sequence derived from HPV16 E7, or a variant thereof, comprise one or more of the following sequences:

SEQ ID NO: 45 (35 aa):
MHGDTPTLHEYMLDLQPETTDLYCYEQLNDSSEEE
SEQ ID NO: 46 (35 aa):
EQLNDSSEEEDEIDGPAGQAEPDRAHYNIVTFCCK
SEQ ID NO: 47 (35 aa):
HYNIVTFCCKCDSTLRLCVQSTHVDIRTLEDLLMG
SEQ ID NO: 48 (23 aa):
IRTLEDLLMGTLGIVCPICSQKP

optionally wherein the polypeptide is capable of eliciting an immune response or is immunostimulatory as defined above.

In some embodiments, the polypeptide comprises two or more peptide fragments each consisting of a sequence derived from HPV16 E7 or a variant thereof, wherein the polypeptide stimulates a T cell response. In some embodiments said two or more peptide fragments consist of one or more of the sequences SEQ ID NOs: 45 to 48.

The peptide fragments may appear within the polypeptide in any order.

As discussed previously, one or more of these sequences is derived from a tumour antigen protein and thus may only have a partial sequence identity therewith as outlined above. As discussed previously, the nucleic acid sequences (for either DNA, RNA, or a mixture of both) can be readily derived by the skilled person).

As discussed previously, the polypeptide may comprise one or more overlapping sequences. As a further illustrative example, if the polypeptide comprises a first peptide fragment having a first sequence, the first sequence being SEQ ID NO: 8, and the polypeptide comprises a second peptide fragment having a second sequence, the second sequence being SEQ ID NO: 9, then the polypeptide would have one overlapping sequence which is the same in each peptide fragment. In this instance, the overlapping sequence would be the following amino acid sequence:

DHRISTFKNWPFLEG

One or more protease cleavage site sequences are located between each of the two or more peptide fragments of the polypeptide of the present invention. In a preferred embodiment, the one or more protease cleavage site sequences are cleavage site sequences of a protease present in the target or host or subject or patient to whom the polypeptide is administered, such that the polypeptide may be cleaved within the host into its peptide fragments. Said protease may act extracellularly or, more preferably, intracellularly. Said protease may be a non-host protease delivered in combination with the polypeptide or its encoding nucleic acid. More preferably, said protease is a host protease. A host protease may be constitutively present, present only upon induction, or otherwise.

In some embodiments, said one or more protease cleavage site sequence(s) may be a Factor Xa digestion site, optionally Ile-Glu-Gly-Arg. This sequence is cleaved after the Arg. In some embodiments, said one or more protease cleavage site sequence(s) may be the HRV 3 C protease, optionally Leu-Glu-Val-Leu-Phe-Gln/Gly-Pro wherein cleavage occurs between the glutamyl and glycyl residues. In some embodiments, said one or more protease cleavage site sequence(s) may be the HIV protease. In some embodiments, said one or more protease cleavage site sequence(s) may be the metalloproteinases. In some embodiments, said one or more protease cleavage site sequence(s) may be tryptases. In some embodiments, said one or more protease cleavage site sequence(s) may be other proteases such as cathepsins (S, L and B etc), CD13 (human aminopeptidase N).

In some embodiments, said one or more protease cleavage site sequence(s) may be the cleavage site sequence of a cathepsin. In a more preferred embodiment, the one or more protease cleavage site sequence(s) is a cleavage site sequence of cathepsin S. Cathepsin S recognises and cleaves at a number of amino acid sequences, any of which could be used in the present invention, including but not limited to Arg-Cys-Gly-Leu, Thr-Val-Gly-Leu, Thr-Val-Gln-Leu, X-Asn-Leu-Arg, X-Pro-Leu-Arg, X-Ile-Val-Gln, and X-Arg-Met-Lys. In some embodiments, the one or more protease cleavage sites may be any combination of those mentioned above, in any number. As an illustrative example, the polypeptide may comprise six peptide fragments, each separated by one or more protease cleavage sites, wherein the one or more protease cleavage sites comprise four cathepsin S cleavage sites, a tryptase cleavage site, and a metalloproteinase (or metalloprotease) cleavage site.

In a preferred embodiment of the present invention, X-Arg-Met-Lys is the protease cleavage site sequence, more preferably Leu-Arg-Met-Lys (‘LRMK’, SEQ ID NO: 17) is the protease cleavage site sequence. LRMK is a preferred recognition site substrate of cathepsin S (Xu et al., 2009; Kallinteris et al., 2005). In some embodiments, the protease cleavage site sequence comprises the LRMK sequence SEQ ID NO: 17. In some embodiments, the protease cleavage site sequence consists of the LRMK sequence SEQ ID NO: 17.

In one embodiment of the present invention, the Leu-Arg-Met-Lys ‘LRMK’ cleavage site sequence of cathepsin S is used as the one or more protease cleavage site sequence(s), having amino acid sequence:

SEQ ID NO: 17 (4 aa):
LRMK

CD8⁺ T cells (also ‘Cytotoxic T Lymphocytes’, ‘CTLs’) target and lyse diseased and/or infected cells. Traditionally, MHC class I molecules are understood to present fragments of intracellular origin for CD8+ T cell recognition and activation; for example, a cancerous cell may present fragmented products of proteasomal digestion of aberrantly expressed, intracellular proteins on MHC class I cells. CD4+ T cells assist in the activation and expansion of other immune cells, including T cells and B cells. Traditionally, MHC class II molecules are understood to present, to CD4+ T cells, fragments of extracellular origin which have been internalised by antigen-presenting cells for presentation. More recently, cross-presentation has been shown known to occur in addition to these traditional pathways, whereby internalised extracellular fragments may be presented on MHC class I molecules, and vice versa.

The peptide fragments of the present invention, having been cleaved by a protease, may be processed and presented, for example via MHC class I and class II molecules, to cells of the immune system. Amino acid sequences derived from the peptide fragments of the present invention stimulate CD8⁺ and CD4⁺ T cells via their presentation via MHC class I and class II molecules, respectively.

In some embodiments, the polypeptide of the invention is very effective at simulating the T cell response. In some embodiments, the polypeptide stimulates the CD8+ T cell response. In some embodiments, the polypeptide stimulates the CD4+ T cell response. In some embodiments, the polypeptide of the invention stimulates both the CD8+ and CD4+ T cell response.

The polypeptide of the invention comprises overlapping peptide fragments, which further strengthens the T cell response (Zhang et al., 2009). Further, the use of overlapping peptides more comprehensively represents the range of potential T cell epitopes.

Genetic variation in T cell receptor and MHC repertoires within a population mean there may exist population-wide variation in the sequences presented to and/or recognised by CD4⁺ and/or CD8⁺ T cells. The multiple and overlapping peptide fragments of the present invention compensate this variation via the ability to tile, or provide greater coverage of, one or more epitopes and by providing alternative options for immune recognition.

In one exemplary embodiment, the polypeptide of the present invention has the following sequence, including peptide fragments derived from human survivin isoform 1:

(287 aa)
SEQ ID NO: 18
MGAPTLPPAWQPFLKDHRISTFKNWPFLEGLRMKDHRISTFKNWPF
LEGCACTPERMAEAGFIHLRMKACTPERMAEAGFIHCPTENEPDL
AQCFFLRMKPTENEPDLAQCFFCFKELEGWEPDDDPIELRMKFKE
LEGWEPDDDPIEEHKKHSSGCAFLSVKLRMKEHKKHSSGCAFLSV
KKQFEELTLGEFLKLRMKQFEELTLGEFLKLDRERAKNKIAKETN
NKLRMKRERAKNKIAKETNNKKKEFEETAEKVRRAILRMKKEFEE
TAEKVRRAIEQLAAMD

In one exemplary embodiment, the polynucleotide encoding the polypeptide of the present invention has the following sequence (in this exemplary embodiment, the sequence having two restriction endonuclease sites added at the 5′ terminal—Bam HI marked by bold type and Nde I marked by underlined type—and also having one restriction endonuclease site added at the 3′ terminal—Xho I marked by underlined type):

(879 bp)
SEQ ID NO: 19
Ggatcccatatgggtgcaccaactcttcctccagcatggcaacct
ttcctgaaggatcatcgtatctctactttcaagaactggccattc
ctggaaggtctgcgtatgaaggatcaccgtatctctactttcaag
aactggccattccttgagggttgtgcttgtactcctgagcgtatg
gctgaggctggtttcatccacctgcgtatgaaggcttgcactcct
gaacgtatggctgaagctggtttcatccactgtccaactgagaac
gagcctgatctggcacaatgcttcttccttcgtatgaagcctact
gagaacgaacctgatctggctcagtgcttcttctgcttcaaggaa
cttgagggttgggagcctgatgatgatccaatcgagctgcgtatg
aagttcaaggagctggaaggttgggagcctgatgatgatcctatc
gaggagcacaagaagcgctcttctggttgtgctttcctgtctgtc
aaactgcgtatgaaggagcacaagaagcactcttctggttgtgct
ttcctgtctgtcaagaagcagttcgaagaactgactctgggtgag
ttcctgaagctgcgtatgaagcagttcgaggagctgactctgggt
gagttcctgaagctggatcgtgaacgtgctaagaacaagatcgct
aaggagactaacaacaagctgcgtatgaagcgtgagcgtgctaag
aacaagatcgctaaggagactaacaacaagaagaaggagttcgag
gagactgctgagaaggttcgtcgtgctatccttcgtatgaagaag
gagttcgaggagactgctgagaaggttcgtcgtgctatcgagcag
ctggctgccatggactaactcgag

It will be appreciated by the skilled reader that in the abovementioned embodiment, there are 9 peptide fragments, with 8 overlapping sequences, each of the peptide fragments being separated by an LRMK linker. In the abovementioned embodiment, the full sequence of human survivin is covered by the 9 peptide fragments. For the purposes of the present invention, the order of the peptide fragments does not matter. For the purposes of the present invention, the number of peptide fragments must be two or more. It should be noted that these peptide fragments are derived from survivin, the meaning of ‘derived from’ being explained in detail above. These peptide fragments may also be derived from variants of survivin or may be variants of the fragments derived from survivin, as outlined above.

In an embodiment of the invention, the polypeptide comprises SEQ ID NOs: 8, 9, 10, 11, 12, 13, 14, 15, and 16 each separated by an LRMK protease cleavage site. In an embodiment of the invention, the polypeptide comprises a variant (as defined above) of one or more of SEQ ID NOs: 8, 9, 10, 11, 12, 13, 14, 15, and 16 each separated by an LRMK protease cleavage site.

In another exemplary embodiment, the polypeptide of the present invention has the following sequence:

SEQ ID NO: 49 (140 aa):
MHGDTPTLHEYMLDLQPETTDLYCYEQLNDSSEEELRMKEQLNDS
SEEEDEIDGPAGQAEPDRAHYNIVTFCCKLRMKHYNIVTFCCKCD
STLRLCVQSTHVDIRTLEDLLMGLRMKIRTLEDLLMGTLGIVCPI
CSQKP

In one exemplary embodiment, the polynucleotide encoding the polypeptide of the present invention has the following sequence:

SEQ ID NO: 50 (423 bp):
ATGCATGGTGATACCCCGACCCTGCATGAATATATGCTGGATCTG
CAACCGGAAACCACCGATCTGTATTGTTATGAGCAGCTGAATGAT
AGCAGCGAAGAGGAATTACGCATGAAGGAACAGCTGAACGATTCA
AGCGAAGAAGAGGACGAAATTGACGGTCCGGCAGGTCAGGCAGAA
CCGGATCGTGCACATTACAACATTGTTACCTTTTGTTGCAAACTG
AGAATGAAACACTACAATATCGTGACCTTCTGCTGTAAATGTGAT
AGCACCCTGCGTCTGTGTGTTCAGAGCACCCATGTTGATATTCGT
ACATTAGAGGACCTGCTGATGGGCCTGCGGATGAAAATTCGTACC
CTGGAAGACCTGTTAATGGGCACCCTGGGTATTGTTTGTCCGATT
TGTAGCCAGAAACCGtaa

It will be appreciated by the skilled reader that in the abovementioned embodiment, there are 4 peptide fragments, with 3 overlapping sequences, each of the peptide fragments being separated by an LRMK linker. For the purposes of the present invention, the order of the peptide fragments does not matter. For the purposes of the present invention, the number of peptide fragments must be two or more. It should be noted that these peptide fragments are derived from survivin, the meaning of ‘derived from’ being explained in detail above. These peptide fragments may also be derived from variants of survivin or may be variants of the fragments derived from survivin, as outlined above.

In an embodiment of the invention, the polypeptide comprises SEQ ID NOs: 45, 46, 47, and 48, each separated by an LRMK protease cleavage site. In an embodiment of the invention, the polypeptide comprises a variant (as defined above) of one or more of SEQ ID NOs: 45, 46, 47, and 48 each separated by an LRMK protease cleavage site.

By the same logic, a polypeptide of the present invention may be constructed and/or expressed for use in mice. In one embodiment, the polypeptide comprises peptide fragments derived from mouse survivin, such that the polypeptide covers the full mouse survivin sequence, optionally by means of overlapping sequences. In one embodiment, the polypeptide has the following sequence, having peptide fragments derived from mouse survivin:

SEQ ID NO: 51 (279 aa):
MHHHHHHGAPALPQIWQLYLKNYRIATFKNWPFLEDLRMKNYRIA
TFKNWPFLEDCACTPERMAEAGFIHLRMKCACTPERMAEAGFIHC
PTENEPDLAQCFFCLRMKCPTENEPDLAQCFFCFKELEGWEPDDN
PIELRMKFKELEGWEPDDNPIEEHRKHSPGCAFLTVKLRMKEHRK
HSPGCAFLTVKKQMEELTVSEFLKLDLRMKKQMEELTVSEFLKLD
RQRAKNKIAKETNNKLRMKRQRAKNKIAKETNNKQKEFEETAKTT
RQSIEQLAA

In some embodiments, the polypeptide further comprises a tag. The tag may be any tag known to those skilled in the art. For example, the tag may be a myc-tag, a HIS-tag, a FLAG-tag, a GFP (or any other recombinant fluorescent protein related thereto), an HA-tag, a GST-tag, or a V5 tag. The tag may be useful for the detection of the polypeptide in a sample or otherwise for isolation and purification techniques applied during the manufacture of the polypeptide. Antibodies recognising tags are widely known in the art and widely available for purchase.

In some embodiments, the polypeptide is provided as a polynucleotide (either DNA, RNA, or a mixture of both) encoding said polypeptide. Such a polynucleotide can be used in place of the polypeptide in any of the methods of the invention. For example, a polynucleotide encoding the polypeptide can be co-administered with an immuno-oncology agent to a subject, and once administered will cause expression of the polypeptide of the invention such that effectively the polypeptide has been administered to the subject.

In some embodiments, the polypeptide may be a recombinant polypeptide. In the following passages, the polypeptide described above having sequence SEQ ID NO: 18 will be referred to as ‘S-polypeptide’. The terms ‘S-polypeptide’, ‘ROP-Survivin’, and ‘Survivin ROP’ can be used interchangeably herein. The polypeptide described above having sequence SEQ ID NO: 49 will be referred to as ‘ROP-HPV’.

In some embodiments, the immuno-oncology agent is a TNFR Superfamily agonist. The TNFR Superfamily agonist can be any molecule, antibody or fragment thereof, peptide, polypeptide, or protein which activates, upregulates, or stimulates a member of the TNFR Superfamily. As an illustrative example, the agonist may act at one or more of the following targets: HVEM, CD40, OX40, 4-1BB, CD30, GITR, TNFR2, and/or DR3. The TNFR Superfamily agonist may agonise the TNFR Superfamily member directly, or it may allosterically enhance the action of the action of other TNFR Superfamily ligands, or both. Suitable agonists can be readily found by the skilled person on the IUPHAR database (www.guidetopharmacology.org). In some embodiments, the TNFR Superfamily agonist is a HVEM agonist, a CD40 agonist, a OX40 agonist, a 4-1EE agonist, a CD30 agonist, a GITR agonist, a TNFR2 agonist, and/or a DR3 agonist. A polypeptide of the invention, when co-administered with any of the above TNFR Superfamily agonists, increases said agonists potency and/or maximal efficacy.

In some embodiments, the TNFR Superfamily agonist is a 4-1EE agonist which acts to enhance, upregulate, or stimulate the action of 4-1BE. 4-1BB has been studied as a potential therapeutic target in immune therapies for the treatment of cancer. The 4-1BB agonist may be a peptide or fragment thereof, a glycoprotein or fragment thereof, an antibody or fragment thereof, or a small molecule. Several 4-1 BB agonists have been demonstrated in the art. As illustrative examples, the following molecules are known 4-1EE agonists: 4-1EE ligand, utomilumab, and urelumab. Any 4-1EE agonist, including those listed above, can be administered in combination with the polypeptide (e.g. S-polypeptide or ROP-HPV) of the present invention.

In some embodiments, the immuno-oncology agent is a checkpoint inhibitor acting at PD-1. PD-1 has been studied as a potential therapeutic target for the treatment of cancer. The checkpoint inhibitor may be a peptide or fragment thereof, a glycoprotein or fragment thereof, an antibody or fragment thereof, or a small molecule. Several checkpoint inhibitors acting at PD-1 have been demonstrated in the art. As illustrative examples, the following molecules are known PD-1 inhibitors: AUNP-12, pembrolizumab, tislelizumab, spartalizumab, nivolumab, and cemiplimab. Any checkpoint inhibitor, including those listed above, can be administered in combination with the polypeptide of the present invention (e.g. S-polypeptide or ROP-HPV).

In other embodiments, the immuno-oncology agent is a checkpoint inhibitor acting at PDL1. PDL1 has been studied as a potential therapeutic target for the treatment of cancer. The checkpoint inhibitor may be a peptide or fragment thereof, a glycoprotein or fragment thereof, an antibody or fragment thereof, or a small molecule. Several checkpoint inhibitors acting at PDL1 have been demonstrated in the art. As an illustrative example, atezolizumab is known as a PDL1 inhibitor. Any checkpoint inhibitor, including those listed above, can be administered in combination with the polypeptide of the present invention (e.g. S-polypeptide or ROP-HPV).

In other embodiments, the immuno-oncology agent is a checkpoint inhibitor acting at CTLA-4. CTLA-4 has been studied as a potential therapeutic target for the treatment of cancer. The checkpoint inhibitor may be a peptide or fragment thereof, a glycoprotein or fragment thereof, an antibody or fragment thereof, or a small molecule. Several checkpoint inhibitors acting at CTLA-4 have been demonstrated in the art. As illustrative examples, ipilimumab and tremelimumab are known as CTLA-4 inhibitors. Any checkpoint inhibitor, including those listed above, can be administered in combination with the polypeptide of the present invention (e.g. S-polypeptide or ROP-HPV).

When administering the polypeptide of the present invention (e.g. S-polypeptide or ROP-HPV), the dosage of said polypeptide will depend on the cancer to be treated, the severity and course thereof, whether said treatment is preventative, the patient's clinical profile and history, and the skilled medical practitioner's judgement, experience, and discretion. The treatment may be administered only once, or a plurality of times over the course of treatment until the desired outcome is achieved. In this instance the desired outcome would be the shrinkage or elimination of cancerous tumours. In some embodiments, each administration of the polypeptide comprises between 1 to 2000 μg·kg⁻¹ of the polypeptide, preferably 1 to 1000 μg·kg⁻¹, or 1 to 100 μg·kg⁻¹, more preferably 5 to 20 μg·kg⁻¹. In some embodiments, the amount of polypeptide in each administration may be between 1 μg to 10000 μg, preferably between 100 μg and 2000 μg, preferably between 250 μg and 1000 μg. The appropriate amount of such a polypeptide (i.e. the polypeptide of the invention e.g. S-polypeptide or ROP-HPV) for administration to a particular subject can be derived from routine experimentation using techniques known to the skilled person.

The invention described herein comprises the co-administration of the polypeptide e.g. S-polypeptide or ROP-HPV and an immuno-oncology agent at an amount effective for the therapy of cancer whilst avoiding the toxic side effects associated with both TNFR Superfamily agonists and checkpoint inhibitors. In some embodiments, the TNFR Superfamily agonist is a 4-1BB agonist, and the 4-1BB agonist is administered before, after, or at the same time as the polypeptide at a dose non-toxic to the subject. 4-1 BB agonists are known to be toxic to humans at doses above a certain threshold. It has been shown that the efficacy of 4-1BB agonists are proportional to their toxicity (see Qi, X., Li, F., Wu, Y. et al. (2019)). Thus, utomilumab has low efficacy and low toxicity, whilst urelumab is highly efficacious but causes liver toxicity at doses of 0.3 mg·kg⁻¹ or higher, and causes severe liver toxicity at doses greater than or equal to 1 mg·kg⁻¹ (Timmerman et al. (2020); Segal et al. (2017)). A recent study placed the maximum tolerated dose of urelumab in human patients at only 0.1 mg·kg⁻¹ (Timmerman et al. (2020)); at such doses, efficacy is low.

In the present invention, co-administration of the polypeptide of the invention e.g. S-polypeptide with a 4-1 BB agonist has potent anti-tumour activity, even when the 4-1 BB agonist is administered at a dose non-toxic to humans, or below 1 mg·kg⁻¹ optionally below 0.3 mg·kg⁻¹. In some embodiments the polypeptide e.g. S-polypeptide is co-administered with a dose of 4-1EE agonist, the dose being less than 10 mg·kg⁻¹, preferably less than 1 mg·kg⁻¹, less than 0.6 mg·kg⁻¹, less than 0.3 mg·kg⁻¹, less than 0.1 mg·kg⁻¹. It will be understood by the skilled reader that any of the doses of the 4-1EE agonists listed here can be combined with the administration of the polypeptide e.g. S-polypeptide at the dosages outlined above, namely between 1 to 2000 μg·kg⁻¹ of the polypeptide, preferably 1 to 1000 μg·kg⁻¹, or 1 to 100 μg·kg⁻¹, more preferably 5 to 20 μg·kg⁻¹. In some embodiments, the amount of polypeptide in each administration may be between 1 μg to 10000 μg, preferably between 100 μg and 2000 μg, preferably between 250 μg and 1000 μg. Likewise, it will be understood that the dosage of 4-1BB agonist depends upon the chosen agonist and its toxicity profile, the cancer to be treated, the severity and course thereof, whether said treatment is preventative, the patient's clinical profile and history, and the skilled medical practitioner's judgement, experience, and discretion, and that the dosage regime for any particular situation will be readily derivable to those skilled in the art. Utomilumab has not been shown to have dose-limiting liver toxicity and thus can be administered at a higher dose than urelumab. However, the efficacy of utomilumab may be improved by co-administration with the polypeptide of the present invention e.g. S-polypeptide. The co-administration of any TNFR Superfamily agonist with the polypeptide e.g. S-polypeptide or ROP-HPV results in a more efficacious treatment for cancer.

In some embodiments, the polypeptide of the invention (e.g. S-polypeptide or ROP-HPV) is co-administered with a checkpoint inhibitor. The checkpoint inhibitor may be an inhibitor of PD-1, and may be administered before, at the same time, or after the administration of the polypeptide (e.g. S-polypeptide or ROP-HPV) and/or at a dose non-toxic to the subject. It will be understood by the skilled reader that any of the doses of the checkpoint inhibitors listed here can be combined with the administration of the polypeptide (e.g. S-polypeptide or ROP-HPV) at the dosages outlined above, namely between 1 to 2000 μg·kg⁻¹ of the polypeptide, preferably 1 to 1000 μg·kg⁻¹, or 1 to 100 μg·kg⁻¹, more preferably 5 to 20 μg·kg⁻¹. In some embodiments, the amount of polypeptide in each administration may be between 1 μg to 10000 μg, preferably between 100 μg and 2000 μg, preferably between 250 μg and 1000 μg.

Likewise, it will be understood that the dosage of checkpoint inhibitor depends upon the chosen inhibitor and its toxicity profile, the cancer to be treated, the severity and course thereof, whether said treatment is preventative, the patient's clinical profile and history, and the skilled medical practitioner's judgement, experience, and discretion, and that the dosage regime for any particular situation will be readily derivable to those skilled in the art. For a review of common checkpoint inhibitors and their toxicity profiles, see Spiers, Laura et al. “Toxicities associated with checkpoint inhibitors—an overview.” Rheumatology (Oxford, England) vol. 58, Suppl 7 (2019). The co-administration of any checkpoint inhibitor with the S-polypeptide results in a more efficacious treatment for cancer.

The administration of the polypeptide of the invention (e.g. S-polypeptide or ROP-HPV), and either the TNFR Superfamily agonist(s) or the checkpoint inhibitor(s) may be repeated periodically. In some embodiments the administration of the polypeptide of the invention (e.g. S-polypeptide or ROP-HPV) and the immuno-oncology agent(s) (TNFR Superfamily agonist(s) or checkpoint inhibitor(s)) is repeated daily, every 2, 3, 4, 5, 6, 7, 8, 9, 10, 11, 12, 13, 14 or more days. In some embodiments the administration of the polypeptide of the invention (e.g. S-polypeptide or ROP-HPV) and the immuno-oncology agent(s) (TNFR Superfamily agonist(s) or checkpoint inhibitor(s)) is repeated daily, weekly, fortnightly, every 3 weeks, monthly, or quarterly.

In one embodiment, a composition or pharmaceutical composition is provided comprising the polypeptide of the invention (e.g. S-polypeptide or ROP-HPV), and/or an immuno-oncology agent as defined herein above. In some embodiments, the composition or pharmaceutical composition additionally comprises a pharmaceutically acceptable delivery vehicle. The polypeptide of the invention (e.g. S-polypeptide or ROP-HPV) and/or the polynucleotide of the invention may be administered to a subject by means of said delivery vehicle. Likewise, the immuno-oncology agents may also be delivered in the same or different pharmaceutically acceptable delivery vehicles. In one embodiment, the pharmaceutically acceptable delivery vehicle is a viral vector, for example—but not limited to—an adenovirus, an adeno-associated virus, MVA, HSV. In another embodiment, the pharmaceutically acceptable delivery vehicle is a bacterial vector, for example—but not limited to—Listeria spp., Salmonella spp. In another embodiment, the pharmaceutically acceptable delivery vehicle is a plasmid, a nanoparticle, a lipoparticle, a polymeric particle, or a virus-like particle.

In one embodiment, the composition or pharmaceutical composition optionally comprises one or more pharmaceutically acceptable carriers (or excipients). Examples of such suitable excipients for the different forms of pharmaceutical compositions described herein may be found in the “Handbook of Pharmaceutical Excipients”, 2nd Edition, (1994), Edited by A Wade and PJ Weller. The composition or pharmaceutical composition may comprise one or more additional components. In one embodiment, the carrier is suitable for injectable delivery. In another embodiment, the carrier is suitable for pulmonary delivery. In another embodiment, the carrier is suitable for oral delivery. In one embodiment, the composition or pharmaceutical composition additionally comprises a therapeutically active agent. In one embodiment, the composition or pharmaceutical composition optionally comprises one or more pharmaceutically acceptable adjuvants. Suitable adjuvants will be understood by the skilled person. In one embodiment, the pharmaceutically acceptable adjuvant could be selected from the non-exhaustive list of: monophosphoryl lipid A (MPL), Alum, AS501, montanide, CpG, ICLC. In one embodiment, the composition or pharmaceutical composition is optionally admixed with one or more pharmaceutically acceptable diluents, excipients or carriers.

In some embodiments, the cancer may be malignant or benign and primary or secondary. The present invention may be used either as a preventative treatment or a curative treatment. In some embodiments, the cancer is any cancer which expresses survivin. Illustrative examples of such cancers comprise cancers of the lung, oesophagus, breast, pancreas, ovaries, uterus, skin, intestines, liver, stomach, bladder, kidney, head and neck, prostate, colorectal and oral cancers, as well as haematological cancers such as acute myeloid leukaemia and acute lymphocytic leukaemia. It would be routine to the person skilled in the art to ascertain whether the cancer is treatable with the co-administration of the polypeptide of the invention (e.g. S-polypeptide or ROP-HPV) and an immuno-oncology agent as described herein.

Also encompassed in the invention is a composition for use in the treatment of cancer, wherein the composition comprises a polypeptide of any of the aforementioned embodiments (e.g. S-polypeptide or ROP-HPV), and the method of treating cancer comprises co-administration of the polypeptide and an immuno-oncology agent as outlined in any of the previous embodiments.

Also encompassed in the invention is a polypeptide for use in the treatment of cancer, wherein the polypeptide is a polypeptide of the invention as described in detail herein, and the method of treating cancer comprises co-administration of the polypeptide and an immuno-oncology agent as outlined in any of the previous embodiments.

In some embodiments, the invention provides a composition for use in a combination therapy for the treatment of cancer, wherein the composition comprises the polypeptide of any of the aforementioned embodiments (e.g. S-polypeptide or ROP-HPV), and the combination therapy comprises the co-administration of the polypeptide and an immuno-oncology agent as outlined in any of the previous embodiments.

Also encompassed in the invention is an immuno-oncology agent for use in a method of treating cancer, wherein the immune oncology agent comprises the 4-1BB agonist or the PD-1 inhibitor of any of the aforementioned embodiments, and the method of treating cancer comprises co-administration of the immuno-oncology agent with polypeptide (e.g. S-polypeptide) as outlined in any of the previous embodiments. In some embodiments the invention provides an immuno-oncology agent for use in a combination therapy for the treatment of cancer, wherein the immune oncology agent comprises the 4-1 BB agonist or the PD-1 inhibitor of any of the aforementioned embodiments, and the combination therapy comprises the co-administration of the immuno-oncology agent with the polypeptide of the invention (e.g. S-polypeptide) as outlined in any of the previous embodiments.

The present invention also encompasses a composition for use in combination with an immuno-oncology agent as outlined in any of the previous embodiments.

Also encompassed in the present invention is a composition for use in a method of treating cancer, wherein the composition comprises the polypeptide of any preceding embodiment (e.g. S-polypeptide or ROP-HPV), wherein the method of treating cancer comprises administering separately, sequentially, or simultaneously, the polypeptide and an immuno-oncology agent as outlined in any of the previous embodiments.

Also encompassed in the present invention is a kit for the treatment of cancer comprising the polypeptide of any of the preceding embodiments (e.g. S-polypeptide or ROP-HPV) and an immuno-oncology agent as described in any of the preceding embodiments outlined above.

Also encompassed in the present invention is a method of determining whether a cancer is suitable for treatment according to the methods of treatment, compositions, and co-administrations of the invention herein described, the method comprising i) administering to a subject or an in vitro sample a polypeptide comprising two or more peptide fragments, wherein a first peptide fragment comprises a first sequence derived from a tumour antigen and wherein a second peptide fragment comprises a second sequence derived from a tumour antigen, further comprising one or more exogenous cathepsin cleavage site sequences located between each of the two or more peptide fragments; ii) administering to a subject or an in vitro sample an immuno-oncology agent; and iii) measuring T cell stimulation in said subject or in vitro sample. T-cell stimulation is a correlate with success of the treatment. Said in vitro sample may be a biopsy, having been isolated from a patient or subject.

The invention is described by reference to the following non-limiting Examples.

### Example 1—Design, Expression, and Purification of Recombinant Overlapping Proteins

### Recombinant Overlapping Peptide Design

Fusion proteins were designed by identifying peptide fragments of between 20 and 35 amino acids in length derived from a protein or proteins of interest and which are to be ultimately liberated intracellularly, following administration of the fusion protein to a subject. In some embodiments, peptide fragments had overlapping sequences, and thus such fusion proteins are known as a ‘recombinant overlapping peptide’ or ‘ROP’. In some embodiments, the peptide fragments together covered the whole sequence of the protein(s) of interest, but in other embodiments only the most immunogenically relevant sections of the protein(s) of interest were represented in the peptide fragments. Peptide fragments were then linked in tandem via the LRMK sequence.

### ROP-Survivin:

A polypeptide vaccine comprising a Recombinant Overlapping Protein (‘ROP’) (SEQ ID NO: 18) was designed using the human survivin protein sequence (SEQ ID NO: 1, Uniprot. identifier O15392-1). This ROP, named ‘ROP-Survivin’, comprises 9 peptide fragments (‘PF’s), each linked to the next via a LRMK cleavage sequence of cathepsin S, such that the PFs can be liberated intracellularly upon digestion by cathepsin S. Each PF is numbered 1 to 9 according to sequential amino acid position within the ROP, with PF1 being the PF most proximate to the N-terminus and PF9 the most proximate to the C-terminus. The sequences of the PFs are as follows:

| Identifier | Amino Acid Sequence |
|---|---|
| PF1 | MGAPTLPPAWQPFLKDHRISTFKNWPFLEG |
| PF2 | DHRISTFKNWPFLEGCACTPERMAEAGFIH |
| PF3 | ACTPERMAEAGFIHCPTENEPDLAQCFF |
| PF4 | PTENEPDLAQCFFCFKELEGWEPDDDPIE |
| PF5 | FKELEGWEPDDDPIEEHKKHSSGCAFLSVK |
| PF6 | EHKKHSSGCAFLSVKKQFEELTLGEFLK |
| PF7 | QFEELTLGEFLKLDRERAKNKIAKETNNK |
| PF8 | RERAKNKIAKETNNKKKEFEETAEKVRRAI |
| PF9 | KEFEETAEKVRRAIEQLAAMD |

PFs 1 to 9 contain sequences from the human survivin protein isoform 1 (SEQ ID NO: 1) and are designed such that the PFs cover the whole of the sequence of said protein, with each PF sharing partial sequences (i.e. having an ‘overlapping sequence’) with at least one other PF. For Example, PF1 contains amino acids 1 to 30 of the human survivin protein, and PF2 contains amino acids 16 to 45 of the human survivin protein such that both contain amino acids 16 to 30 i.e. a so-called ‘overlapping sequence’.

The full sequence of ROP-Survivin is SEQ ID NO: 18.

mROP-Survivin:

A mouse ROP-survivin (‘mROP-survivin’) with peptide fragments derived from mouse survivin was also designed and produced, having 8 PFs with sequences as follows:

| Identifier | Amino Acid Sequence |
|---|---|
| PF1 | GAPALPQIWQLYLKNYRIATFKNWPFLED |
| PF2 | NYRIATFKNWPFLEDCACTPERMAEAGFIH |
| PF3 | CACTPERMAEAGFIHCPTENEPDLAQCFFC |
| PF4 | CPTENEPDLAQCFFCFKELEGWEPDDNPIE |
| PF5 | FKELEGWEPDDNPIEEHRKHSPGCAFLTVK |
| PF6 | EHRKHSPGCAFLTVKKQMEELTVSEFLKLD |
| PF7 | KOMEELTVSEFLKLDRQRAKNKIAKETNNK |
| PF8 | RQRAKNKIAKETNNKQKEFEETAKTTRQSI |
| | EQLAA |

The full sequence of mROP-Survivin is SEQ ID NO: 51.

### ROP-HPV16 E7:

ROP-HPV16 E7 comprises 4 PFs with sequences as follows:

| Identifier | Amino Acid Sequence |
|---|---|
| PF1 | MHGDTPTLHEYMLDLQPETTDLYCYEQLNDSSEEE |
| PF2 | EQLNDSSEEEDEIDGPAGOAEPDRAHYNIVTFCCK |
| PF3 | HYNIVTFCCKCDSTLRLCVQSTHVDIRTLEDLLMG |
| PF4 | IRTLEDLLMGTLGIVCPICSQKP |

PFs 1 to 4 contain sequences from the HPV16 E7 protein (SEQ ID NO: 43) and are designed such that the PFs cover the whole of the sequence of said protein, with each PF sharing partial sequences (i.e. having an ‘overlapping sequence’) with at least one other PF. For Example, PF1 contains amino acids 1 to 35 of the HPV16 E7 protein, and PF2 contains amino acids 26 to 61 of the HPV16 E7 protein such that both contain amino acids 26 to 35 i.e. a so-called ‘overlapping sequence’.

The full sequence of ROP-HPV16 E7 is SEQ ID NO: 49.

### Molecular Cloning

Component gene fragments were designed to correspond to the designed fusion protein, and codon optimized for expression in E. coli. The whole gene sequence of fusion protein was constructed from fragments by PCR, and product was verified via electrophoresis. Product was isolated by horizontal electrophoresis on Agarose recovery gel with 1×TAE buffer. Specific reaction conditions are provided for ROP-Survivin and ROP-HPV16 E7 as follows:

### ROP-Survivin:

In order to construct the designed ROP, we designed and synthesized 22 synthetic gene fragments SEQ ID NOs: 21-42), with codons optimized for expression in E. coli:

| | |
|---|---|
| #1 SEQ ID NO: 21 | GGATCCCATA TGGGTGCACC AACTCTTCCT |
| | CCAGCATGGC AACCTTTCCT GAAGGATCAT |
| #2 SEQ ID NO: 22 | GACCTTCCAG GAATGGCCAG TTCTTGAAAG |
| | TAGAGATACG ATGATCCTTC AGGAAAGGTT |
| #3 SEQ ID NO: 23 | CTGGCCATTC CTGGAAGGTC TGCGTATGAA |
| | GGATCACCGT ATCTCTACTT TCAAGAACTG |
| #4 SEQ ID NO: 24 | CATACGCTCA GGAGTACAAG CACAACCCTC |
| | AAGGAATGGC CAGTTCTTGA AAGTAGAGAT |
| #5 SEQ ID NO: 25 | CTTGTACTCC TGAGCGTATG GCTGAGGCTG |
| | GTTTCATCCA CCTGCGTATG AAGGCTTGCA |
| #6 SEQ ID NO: 26 | GGACAGTGGA TGAAACCAGC TTCAGCCATA |
| | CGTTCAGGAG TGCAAGCCTT CATACGCAGG |
| #7 SEQ ID NO: 27 | GCTGGTTTCA TCCACTGTCC AACTGAGAAC |
| | GAGCCTGATC TGGCACAATG CTTCTTCCTT |
| #8 SEQ ID NO: 28 | ACTGAGCCAG ATCAGGTTCG TTCTCAGTAG |
| | GCTTCATACG AAGGAAGAAG CATTGTGCCA |
| #9 SEQ ID NO: 29 | CGAACCTGAT CTGGCTCAGT GCTTCTTCTG |
| | CTTCAAGGAA CTTGAGGGTT GGGAGCCTGA |
| #10 SEQ ID NO: 30 | CAGCTCCTTG AACTTCATAC GCAGCTCGAT |
| | TGGATCATCA TCAGGCTCCC AACCCTCAAG |
| #11 SEQ ID NO: 31 | GTATGAAGTT CAAGGAGCTG GAAGGTTGGG |
| | AGCCTGATGA TGATCCTATC GAGGAGCACA |
| #12 SEQ ID NO: 32 | AGTTTGACAG ACAGGAAAGC ACAACCAGAA |
| | GAGTGCTTCT TGTGCTCCTC GATAGGATCA |
| #13 SEQ ID NO: 33 | GCTTTCCTGT CTGTCAAACT GCGTATGAAG |
| | GAGCACAAGA AGCACTCTTC TGGTTGTGCT |
| #14 SEQ ID NO: 34 | CCAGAGTCAG TTCTTCGAAC TGCTTCTTGA |
| | CAGACAGGAA AGCACAACCA GAAGAGTGCT |
| #15 SEQ ID NO: 35 | GTTCGAAGAA CTGACTCTGG GTGAGTTCCT |
| | GAAGCTGCGT ATGAAGCAGT TCGAGGAGCT |
| #16 SEQ ID NO: 36 | AGCACGTTCA CGATCCAGCT TCAGGAACTC |
| | ACCCAGAGTC AGCTCCTCGA ACTGCTTCAT |
| #17 SEQ ID NO: 37 | AGCTGGATCG TGAACGTGCT AAGAACAAGA |
| | TCGCTAAGGA GACTAACAAC AAGCTGCGTA |
| #18 SEQ ID NO: 38 | GTCTCCTTAC GATCTTGTTC TTAGCACGCT |
| | CACGCTTCAT TACGCAGCTT GTTGTTAGTC |
| #19 SEQ ID NO: 39 | AACAAGATCG CTAAGGAGAC TAACAACAAG |
| | AAGAAGGAGT TCGAGGAGAC TGCTGAGAAG |
| #20 SEQ ID NO: 40 | CCTCGAACTC CTTCTTCATA CGAAGGATAG |
| | CACGACGAAC CTTCTCAGCA GTCTCCTCGA |
| #21 SEQ ID NO: 41 | TATGAAGAAG GAGTTCGAGG AGACTGCTGA |
| | GAAGGTTCGT CGTGCTATCG AGCAGCTGGC |
| #22 SEQ ID NO: 42 | CTCGAGTTAG TCCATGGCAG CCAGCTGCTC |
| | GATAGCACG |

The whole gene sequence of the ROP was constructed from these gene fragments SEQ ID NOs 21-42 via PCR with Taq DNA polymerase KOD FX, purchased from TOYOBO Co., using the reaction system:

| | Volume (μl) | Final Concentration |
|---|---|---|
| 2x PCR buffer for KOD FX | 25 | 1x |
| 2 mM dTNPs | 10 | 0.4 mM each |
| 10 pmol/μl gene fragment #1 | 1.5 | 0.3 μM |
| 5′ primer | | |
| 10 pmol/μl gene fragment #22 | 1.5 | 0.3 μM |
| 3′ primer | | |
| 1 pmol/μl gene fragments #2- | 2 | |
| #21 inner primers | | |
| KOD FX (1.0 U/ul) | 1 | 1.0 U/50 μl |

| | |
|---|---|
| Autoclaved, distilled water | Up to 50 μl |

and with reference to Nucleic Acids Research, 2004, 32, e98.

The PCR was run with three-step reaction conditions:

| | | | |
|---|---|---|---|
| A - Pre-denature: | 94° C., 2 min; | B - Denature: | 98° C., 10 sec |
| C - Annealing: | 54° C., 30 sec; | D - Extension: | 68° C., 1 min/kb |

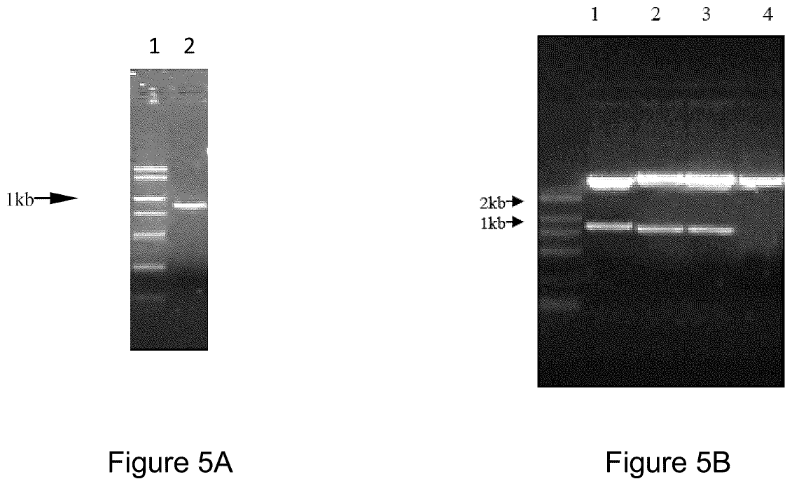

Steps B-D were repeated ×25 cycles for amplification. After amplification, a small amount of EX Taq polymerase was added to extend the reaction at 68° C. for an extra 30 minutes. The synthesized PCR product was verified via electrophoretic detection on a 0.8% Agarose gel (FIG. 5A). The band corresponded to the theoretical 879 bp molecular weight.

The ROP DNA PCR product was isolated on a 0.8% concentration Agarose recovery gel with 1×TAE buffer via horizontal electrophoresis. The agarose gel was prepared and EB added to a concentration of 0.5 μg/ml. The sample was mixed with loading buffer: 10× loading buffer:

| | |
|---|---|
| Ficoll 400 | 20% |
| EDTA pH 8.0 | 0.1M |
| SDS | 1% |
| Bromphenol Blue | 0.25% |

A voltage of 1-5 Volt/cm was applied for 20 minutes, and the bands observed under UV light. To recover the DNA from the gel, a DNA fragment recovery Kit purchased from Axygen Xo. was used according to the kit protocol.

### ROP-HPV16 E7:

A codon-optimised synthetic DNA sequence of a ROP-HPV16E7 is provided as SEQ ID NO: 50.

### Transformation

### ROP-Survivin:

Recovered product was amplified using the Vector system pMD18-T kit, purchased from TAKARA Co., with reaction conditions to a total volume of 30 μl. 5×T4 DNA ligase buffer was made up according to the composition:

| | | |
|---|---|---|
| Tris.Cl (pH 7.6) | 330 | nM |
| MgCl₂ | 33 | mM |
| DTT | 50 | mM |
| ATP | 220 | μM |

Materials were mixed thoroughly and evenly and left at 4° C. overnight.

One 1 ml vial of competent ED60 E. coli cells was removed from −70° C. and thawed on ice for 5 mins. A 1.5 ml centrifuge tube was pre-chilled on ice for 5 mins, and 200 μl competent cells added. 5 μl ligation reaction solution was added, mixed, and placed on ice for 20 mins; the tube was flicked every 10 mins. Cells were screened by overnight 37° C. culture on 2YT culture medium agar plates with 100 mg/ml Ampicillin. 4 white colonies selected from the overnight plate and inoculated into 3 ml 2YT culture medium with 100 mg/ml Ampicillin, incubation at 37° C. overnight with shaking. 1.5 ml of overnight culture was centrifuged for 30 seconds at 13,000 rpm. Supernatant was discarded and cells washed once with STE buffer (0.1 M NaCl 5.85 g; 10 mM Tris.Cl pH 8 1.21 g; 1 mM EDTA pH8) and suspended in 100 μl pre-chilled solution I (50 mM glucose; 25 mM Tris.Cl pH 8; 10 mM EDTA pH 8). 200 μl freshly prepared solution II (0.2 M NaOH; 1% SDS) was added and evenly mixed. 150 μl pre-chilled solution III (5 M KAc 60 mL; HAc 11.5 mL; H2O 28.5 mL) was added and evenly mixed and placed on ice for 15 mins. The solution was centrifuged for 10 mins at 13,000 rpm. Supernatant was transferred to a new centrifuge tube and DNase-free RNase added to a final concentration of 100 μg/ml. The tube was placed on ice for 10 mins. Supernatant was transferred to a new centrifuge tube and 1 ml pre-chilled ethanol added, mixed evenly and left at RT for 5 mins, then centrifuged for 10 mins at 13000 rpm.

Transformation was verified by electrophoresis of the products of a Bam HI+Xho I restriction digest, using 3 μL plasmid DNA, 0.2 μL of each restriction enzyme, 2 μL of 10× buffer, to a total volume of 20 μL, incubated for 2 hours at 37° C. A voltage of 1-5V for 20 minutes and observation under UV confirmed successful transformation through the presence of bands at 879 bp in each lane 1, 2, and 3 (FIG. 5B). Resultant plasmids YN8735 (lane 1), TN8736 (lane 2), YN8737 (lane 3) were retained at −20° C.

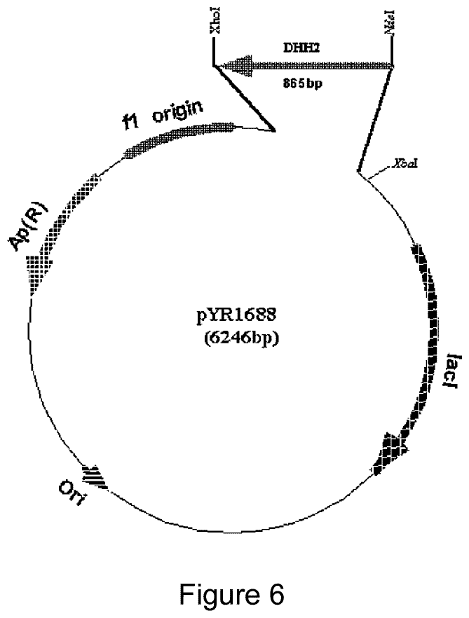

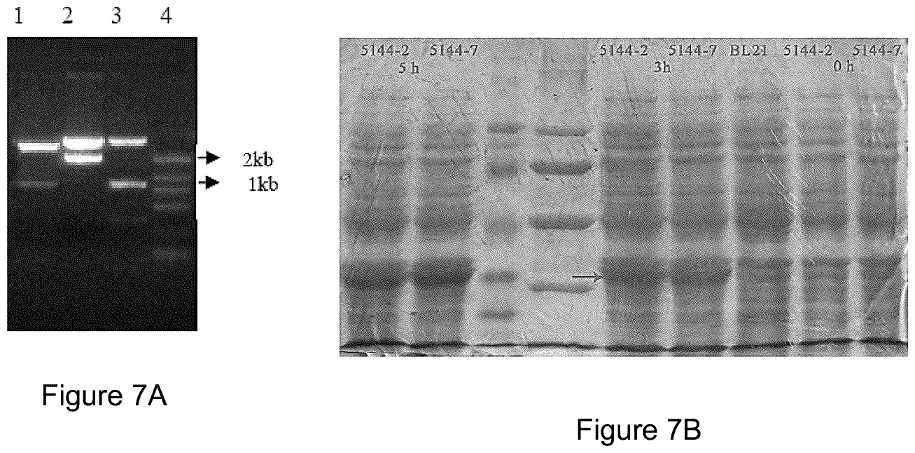

To construct the plasmid for engineering, YN8735 was digested with Ndel and Xhol to a total volume of 40 μl and the cleaved product recovered from a 8% agarose gel using a Kit from Axygen Co. Vector plasmid pET32a was likewise digested with Ndel and Xhol and the digestion products ligated with T4 DNA ligase at 16-18° C. for 12-18 hours. Resulting plasmid pYR1688 (FIG. 6) was produced. pYR1688 was transformed into competent DH5a E. coli and screened on 2YT agar overnight at 37° C. in the presence of 100 mg/ml Ampicillin. Picking of 3 colonies and digesting plasmid DNA therefrom with Nedl and Xhol to a total volume of 40 μL confirmed transformation in colony 1 and 3 (FIG. 7A: lane 1=colony 1; lane 2=colony 2; lane 3=colony 3). Plasmids P1 and P3 (from colonies 1 and 3 respectively) were verified by DNA sequencing carried out by Shanghai Sang Ni Bio-engineering Co. Ltd. Overnight cell cultures of both colonies 1 and 3 (named ‘YN5144’ and ‘YN5145’ respectively) were deposited in a cell bank.

### Protein Expression and Purification

Plasmid P1 was extracted from YN5144 and transformed into competent E. coli EG63 (BL21). Transformed E. coli were screened on 37° C. overnight 2YT agar with 100 mg/ml Ampicillin. Colonies were picked and inoculated separately into 2YT culture with 100 mg/mL Ampicillin at 37° C. with shaking for 12 hours twice, first into 5 ml 2YT+Amp and subsequently into 100 ml 2YT+Amp. 20 ml of this seed cell culture were inoculated into 1000 ml 2YT+Amp and incubated at 37° C. with shaking to OD 0.3-0.5. IPTG was added to final concentration of 1 mM and incubated for 3-5 hours at 30° C. Following induction, 1 L cell culture was centrifuged at 4° C. 5000 rpm for 15 mins. The supernatant was discarded and cells suspended with TE buffer at a ratio of 1 g wet cells:3 ml TE. Cells were sonicated at 15-20 kHz then centrifuged for 15 mins at 10,000 rpm. The supernatant and pellet were collected separately.

Induction and expression were verified by SDS-PAGE (30% Acr/Bis solution; 1.5 mol/L Tris-HCl pH8.8; 0.5 mol/L Tris-HCl pH6.8; Tris-glycine electrophoresis buffer) with a 12% separation gel and 5% condensed gel. 1× loading buffer was added to the sample and the solution boiled for 5 mins before dipped immediately into ice. The solution was centrifuged for 5 mins at 12,000 rpm and 10 μl of supernatant added to each lane. 8 V/cm was used at the condensing stage and 15 V/cm at the separation stage until bromophenol blue reached the end of the gel.

His tag sequence was in the expression vector. The gel was washed in distilled water for 10 minutes, then staining solution (Coomassie Brilliant Blue R250 0.25 g, 91 ml 50% Methanol, 9 ml HAc) added at 37° C. for 2 hours. The staining solution was recovered and the gel moved to de-staining solution (50 ml methanol, 75 mL HAc, add 875 ml distilled water) which was changed every 3 hours until the background showed clear. SDS-PAGE resulted showed expressed recombinant protein at 30 kDa molecular weight (identical to theoretical calculation) expressed at approximately 50% of total proteins after 5 hours of induction with IPTG (FIG. 7B).

The ROP-survivin mainly was detected in the pellet. So the cell pellets were resuspended in buffer (25 mM Tris-HCl, 200 mM NaCl, 8 M urea, 10 mM imidazole, pH8.0) and centrifuged at 20,000 g for 45 min, before applying to the Ni-NTA resin. Washing buffer and elution buffer also contained 8 M urea. For refolding, the eluted proteins were first buffer exchanged to 25 mM Tris-HCl, 200 mM NaCl, 0.5 arginine-HCl, pH 10.5 then to PBS with 10% glycerol using a PD10 column (GE Healthcare Life Sciences, UK).

### ROP-HPV

Recovered product was amplified as above for ROP-Survivin and transformed into competent E. coli for overnight culture. ROP expression was induced by addition of IPTG (final concentration of 0.5 mM). Cells were collected and resuspended in lysis buffer (25 mM Tris-HCl, 200 mM NaCl, 2% Triton X-100, 10 mM imidazole, pH8.0) and were lysed by sonication. Insoluble fractions were separated by centrifugation at 20,000 g for 45 min. His tag sequence was in the expression vector. For expressed soluble protein, Ni-NTA resin was then added to the soluble fractions for 30 min, followed by washing with 30 resin volumes of lysis buffer and eluted with lysis buffer containing 300 mM imidazole. The eluted proteins were dialyzed to PBS buffer with 10% glycerol.

Mouse ROP-Survivin (mROP-Survivin)

A single transformed BL21 (DE3) colony was picked into LB (50 μg/ml Kan) and cultured overnight at 37° C. The overnight culture was then diluted 100 times with fresh LB and cultured until the OD600 reached 0.6. Then IPTG was added to a final concentration of 0.5 mM. The cells were harvested at 16 hr post IPTG induction by centrifugation. The cell pellets were resuspended in lysis buffer (25 mM Tris-HCl, 200 mM NaCl, 2% Triton X-100, 10 mM imidazole, pH8.0) and were lysed by sonication. Insoluble fractions were separated by centrifugation at 20,000 g for 45 min. The insoluble fractions were re-suspended in buffer (25 mM Tris-HCl, 200 mM NaCl, 8 M urea, 10 mM imidazole, pH8.0) and centrifuged at 20,000 g for another 45 min, before applying to the Ni-NTA resin. Washing buffer and elution buffer also contained 8 M urea. For refolding, the eluted proteins were first buffer exchanged to 25 mM Tris-HCl, 200 mM NaCl, pH 8.0 then to PBS with 10% glycerol using a PD10 column (GE Healthcare Life Sciences, UK).

### Example 2—Demonstration of a Combination Approach of a TNFR Superfamily Agonist and a Recombinant Overlapping Peptide Based on Survivin for the Treatment of Cancer

### Materials and Methods

### Mice

A total of 70 female C57BL/6 mice were ordered from Changzhou Kavins Experimental Animal Co. Ltd. The animals were specific pathogen free and approximately 6-7 weeks old upon arrival at Changzhou Niujin Shisong Biotechnology Co., Ltd (‘CBI’). Upon receipt the animals were unpacked and placed in cages. A health inspection was performed on each animal, including evaluation of the coat, extremities and orifices. Each animal was also examined for any abnormal signs in posture or movement.

### Tumor Cell Lines

B16-F10 and B16-GFP-Survivin cells were purchased (both produced by Shanghai South Model Biotechnology Development Company Ltd). B16-GFP-survivin and B16-F10 cells were maintained at 37° C. under 5% CO2 in DMEM medium supplemented with 10% FBS and subsequently cultured within 10 passages before being inoculated into the mice.

### Immunohistochemistry

The expression of GFP-survivin in B16-GFP-survivin was detected by immunohistochemistry (IHC). B16-F10 cells were used as a negative control. Cells grown on cover slips were fixed in 4% paraformaldehyde for 10 min at room temperature. After permeabilization (0.5% Triton X-100 in PBS), the cells were blocked with 0.5% BSA for 30 min and incubated with anti-survivin antibody XA281 NO10 (Produced by CBI) for one hour at room temperature. The cells were washed and incubated for another hour with HRP-conjugated goat anti-mouse IgG (ab6789, 1/500 diluted), and followed by diaminobenzidine staining (DAB). The nuclei were counterstained with haematoxylin.

### Tumor Models

Mice were injected in the flank subcutaneously on Day 0 with 1×10⁵ B16-GFP-Survivin cells mixed with RPMI 1640 medium. Five days after tumor cell inoculation, 70 mice were randomized into 7 groups (10 mice per group) according to body weight. Treatment was started after grouping (5 days after cell inoculation). The groups are indicated in Table 1 below. ROP-Survivin was produced in accordance with Example 1. Monophosphoryl Lipid A adjuvant (MPL) was purchased from Sigma Aldrich, Dorset, UK (catalogue number S6322). The agonist 4-1BB (CD137) antibody (BE0239) was purchased from BioXCell (New Hampshire, USA). The MPL as provided contains 0.5 mg of MPL, and this was diluted into 2 ml of PBS such that the concentration of MPL was 0.25 mg·ml⁻¹. ROP-Survivin, MPL, and/or PBS was administered subcutaneously every 7 days. 4-1 BB antibody was administered intraperitoneal every 3 days.

TABLE 1

| Group | No. | Test Articles and Doses | Injection Site | Dosing Regimen |
|---|---|---|---|---|
| 1 | 10 | 100 μg ROP-Survivin + 100 μl MPL | S.C. | Every 7 days |
| 2 | 10 | 100 μg ROP-Survivin + 100 μl MPL, | S.C. | Every 7 days |
| | | 4-1BB Ab (1.8 mg/kg) | i.p. | Every 3 days |
| 3 | 10 | 100 μg ROP-Survivin + 100 μl MPL, | S.C. | Every 7 days |
| | | 4-1BB Ab (0.6 mg/kg) | i.p. | Every 3 days |
| 4 | 10 | 100 μl MPL + 100 μl PBS | S.C. | Every 7 days |
| 5 | 10 | 100 μl MPL + 100 μl PBS | S.C. | Every 7 days |
| | | 4-1BB Ab (1.8 mg/kg) | i.p. | Every 3 days |
| 6 | 10 | 100 μl MPL + 100 μl PBS, | S.C. | Every 7 days |
| | | 4-1BBAb (0.6 mg/kg) | i.p. | Every 3 days |
| 7 | 10 | PBS | S.C. | Every 7 days |

(S.C. = subcutaneous; i.p. = intraperitoneal)

Tumors were measured every 3 days (length×width) with a caliper. Tumor volume was determined using the formula: ½×D×d² where D is the major axis and d is the minor axis. Mice were sacrificed when tumors reached 2 cm³ or upon ulceration.

On day 23, all of the mice were sacrificed. Serum and splenocytes were collected for testing humoral and cellular immune responses via ELISPOT and ELISA.

### ELISA

Purified human survivin, ROP-survivin (4 μg/ml) or survivin peptides (2 μg/ml) were coated onto flat-bottomed 96-well microtiter plates (Corning-Costar) in PBS overnight at 4° C. The wells were blocked with 5% BSA for one hour at room temperature. This was followed by incubation with mice blood sera (1:10000 diluted in PBS) at room temperature for one hour. Antibody binding was detected by using HRP-conjugated anti-mouse IgG secondary antibody. After washing, the plates were developed by adding 100 μl of TMB substrate solution. The reaction was stopped and the absorbance at 450 nm was measured using a spectrometer according to standard protocols.

### Isolation of Mouse Splenocytes and ELISPOT Assays

Mouse spleens were strained through a mesh and loaded to murine splenocyte separation medium (Solarbio) and centrifuged at 1000 g for 22 minutes before transferring the layered lymphocytes to a new tube with cell culture medium. The cells were washed twice by RPMI 1640 and 2.5×10⁵ splenocytes per well were used for stimulation in ELISPOT assays.

Cd4+ or CD8+ T cells were purified by negative or positive selection using microbeads kit (Miltenyi, Germany) as per the manufacturer's instructions.

Assays were performed using ELISPOT kits (Mabtech, Sweden). Briefly, splenocytes were re-stimulated overnight with 5 μg/well human survivin or ROP-survivin in anti-IFN-γ-Ab precoated plates (Millipore). Cells were discarded, and biotinylated anti-IFN-γ antibody was added for two hours at room temperature, followed by another one hour of incubation at room temperature with alkaline phosphatase (ALP) conjugated streptavidin. After color developed, the reaction was stopped by washing plates with tap water and plates were air-dried. Spots were counted with an ELISPOT reader (CTL).

### Statistical Analysis

Results were expressed as mean±S.E.M. Comparisons were made by Student's t-test or logrank test as appropriate, p<0.05 was considered to be significant.

### Results

### Human Survivin is Highly Expressed in B16-GFP-Survivin Cells

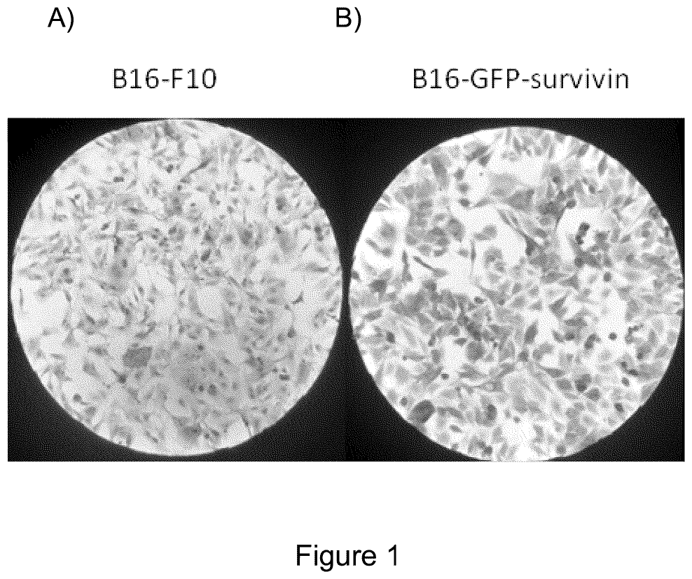

B16-GFP-survivin cells were tested first by IHC using anti-human survivin antibodies. FIG. 1 shows the result of survivin expression in B16-GFP-survivin cells compared to B16-F10 cells. B16-F10 or B16-GFP-survivin cells grown on glass coverslips were fixed and stained with anti-human survivin antibody before examination with a microscope (40× magnification). The nuclei were counterstained with haematoxylin. As shown in FIG. 1, the B16-GFP-survivin cells express human survivin protein at a much higher level than the B16-F10 control cells.

### Combined Treatment of ROP-Survivin and Low Dose Anti-4-1BB Antibody Represses B16-GFP-Survivin Tumor Growth Significantly

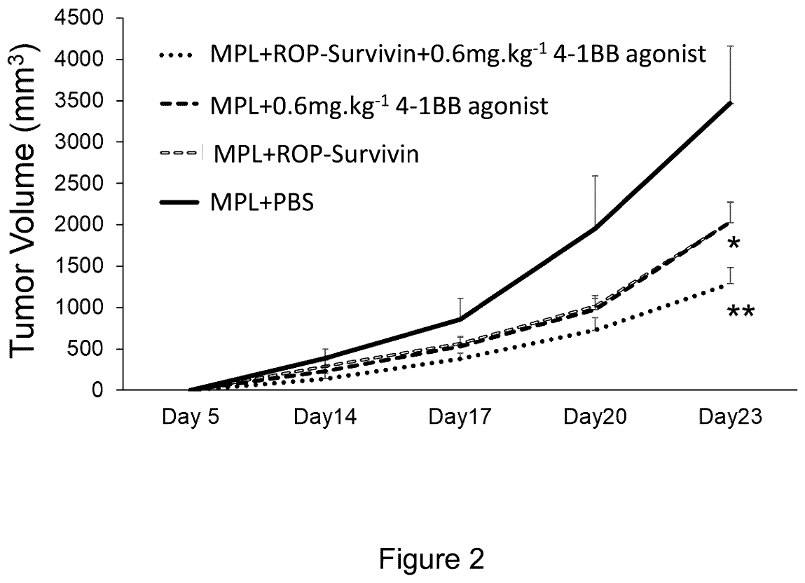

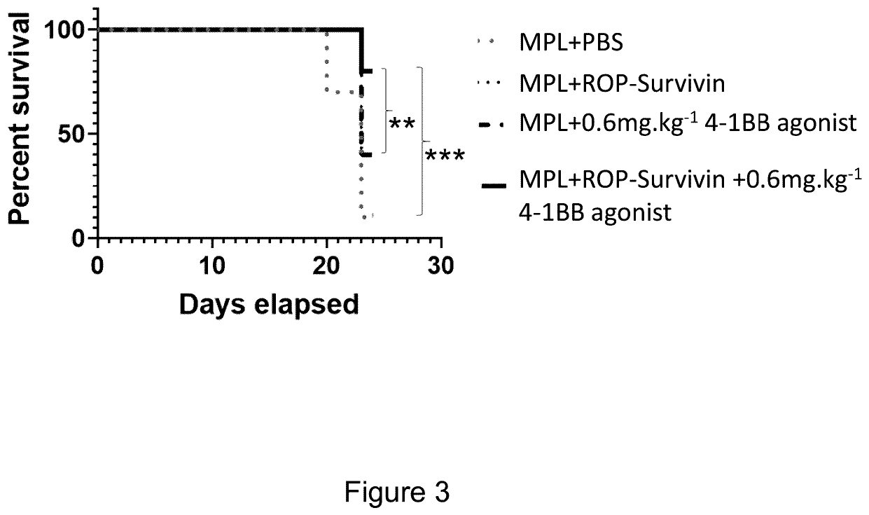

Each C57BL/6 mouse was injected with 1×10⁵ B16-GFP-Survivin cells and randomized into 4 groups (10 mice in each group). Five days after tumor cell inoculation, the mice were treated with PBS+MPL adjuvant, ROP-Survivin (100 μg)+MPL adjuvant, 0.6 mg/kg anti-4-1BB antibody, or ROP-Survivin (100 μg)+MPL combined with 0.6 mg/kg anti-4-1BB antibody. FIGS. 2 & 3 show the tumor volume and survival rate of each group, respectively. FIG. 2 shows the tumor volume of each group measured and recorded every three days. FIG. 3 shows percentage survival of the treated mice.

The tumor volume of ROP-Survivin+MPL vaccinated group is significantly smaller (p<0.05) than the PBS+MPL treated group at 23 days after tumor cell inoculation as shown in FIG. 2. The combined treatment of ROP-Survivin (100 μg)+MPL and 0.6 mg/kg anti-4-1BB antibody significantly repressed the tumor growth (p<0.05) even compared to ROP-Survivin (100 μg)+MPL or anti-4-1 BB antibody treated groups as shown in FIG. 2. FIG. 3 showed that the survival rate of the combined treatment group is much higher than the other groups (ROP-Survivin (100 μg)+4-1BB antibody vs PBS, p<0.01; ROP-Survivin (100 μg)+4-1BB antibody vs ROP-Survivin, p<0.05; ROP-Survivin (100 μg)+4-1BB antibody vs 4-1BB antibody, p<0.05).

### Combined Treatment of ROP-Survivin and High Dose Anti-4-1BB Antibody Didn't Significantly Inhibit Tumor Growth Compared to the ROP-Survivin or Anti-4-1BB Antibody Only Groups

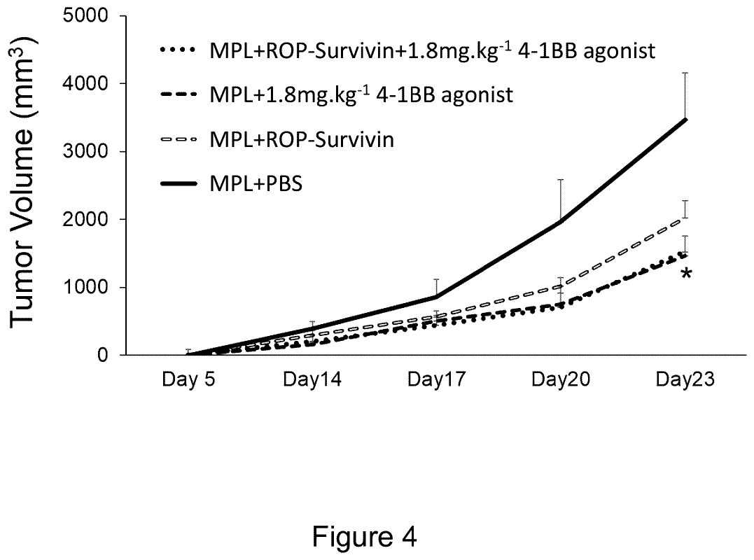

FIG. 4 shows the tumor volume of ROP-Survivin (100 μg)+1.8 mg/kg 4-1 BB antibody, ROP-Survivin (100 μg), 4-1BB antibody or PBS treated control groups. The mean tumor volume of MPL+ROP-Survivin (100 μg) or 1.8 mg/kg anti-4-1BB antibody treated group was much smaller than PBS treated group (p<0.05). The tumor volume of MPL+ROP-Survivin (100 μg)+1.8 mg/kg 4-1BB antibody treated group was very similar to the MPL+1.8 mg/kg 4-1BB antibody group and was not significantly smaller than the MPL+ROP-Survivin (100 μg) group (p>0.05).

We have corroborated that higher doses of 4-1 BB agonists have greater anti-tumor efficacy than lower doses of 4-1BB agonists. We have further demonstrated that administration of ROP-Survivin in combination with low doses of 4-1BB agonist significantly enhances the anti-tumor efficacy over treatment with low doses of 4-1 BB agonist alone. This increase in efficacy allows for the provision of an effective treatment without the need to raise the dose of 4-1BB agonist administered. In other words, potency of 4-1BB agonist is increased. This is advantageous since the high doses of 4-1 BB agonist currently required for effective treatment of cancer in human patients are prohibitively toxic (as discussed herein). Co-administration with ROP-Survivin can increase efficacy without risking dose-dependent toxicity.

### Example 3—Demonstration of a Combination Approach of a Checkpoint Inhibitor and a Recombinant Overlapping Peptide Based on Survivin for the Treatment of Cancer

### Materials and Methods

### Mice

A total of 50 female C57BL/6 mice are ordered from Changzhou Kavins Experimental Animal Co. LTD. The animals are specific pathogen free and approximately 6-7 weeks old upon arrival at CBI. Upon receipt the animals are unpacked and placed in cages. A health inspection is performed on each animal, including evaluation of the coat, extremities and orifices. Each animal is also examined for any abnormal signs in posture or movement.

### Tumor Cell Lines

MC38 tumor cells were purchased from Shanghai Model Organisms. B16-GFP-survivin is maintained at 37° C. under 5% CO2 in DMEM medium supplemented with 10% FBS and subsequently cultured within 10 passages before inoculated into the mice.

### Tumor Models

Each mouse was injected in the flank subcutaneously on Day 0 with 3×10⁵ MC38 cells mixed with RPMI 1640 medium. Five days after tumor cell inoculation, 50 mice were randomized into 5 groups (10 mice per group) according to body weight and immunized with mouse ROP-Survivin (‘mROP-Survivin’), produced in accordance with Example 1, and/or treated with anti-PD-1 antibody according to the Table 3 below.

Monophosphoryl Lipid A adjuvant (MPL) was purchased from Sigma. Anti-PD1 antibody was purchased from BioXcell. ROP-Survivin, MPL, and/or PBS was administered subcutaneously every 7 days. anti-PD-1 antibody was administered intraperitoneal every 3 days.

TABLE 3

| Group | No. | Test Articles and Doses | Injection Site | Dosing Regimen |
|---|---|---|---|---|
| 1 | 10 | 200 μg mROP-Survivin + 100 μl MPL | S.C. | Every 7 days |
| 2 | 10 | 200 μg mROP-Survivin + 100 μl MPL + | S.C. | Every 7 days |
| | | anti-PD-1 Ab (2 mg/kg) | i.p. | Every 3 days |
| 3 | 10 | anti-PD-1 Ab(2 mg/kg) | i.p. | Every 3 days |
| 4 | 10 | 100 μl MPL + 100 μl PBS | S.C. | Every 7 days |
| 5 | 10 | PBS | S.C. | Every 7 days |

Tumors are measured every 3 days (length×width) with a caliper. Tumor volume is determined using the formula: ½×D×d² where D is the major axis and d is the minor axis. Mice are sacrificed when tumors reached 2 cm³ or upon ulceration.

Body weight is measured every 3 days.

On day 14, all the mice are sacrificed. Serum and splenocytes are collected for testing humoral and cellular immune responses via ELISPOT. Mouse splenocytes are isolated and ELISPOT assays carried out according to Example 2.

### Statistical Analysis

Results are expressed as mean±S.E.M. Comparisons are made by Student's t-test or logrank test as appropriate, p<0.05 is considered to be significant.

### Results

Combined Treatment of mROP-Survivin (200 μg/Mouse) and Anti-PD-1 Antibody (2 mg/kg) Significantly Reduced the Growth of MC38 Tumor in Mice

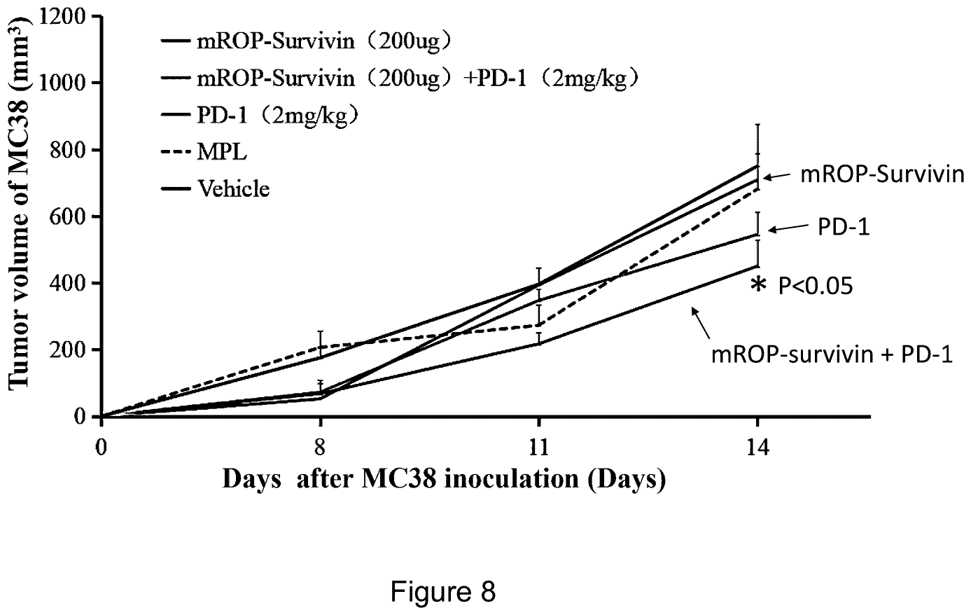

As can be seen from FIG. 8, mouse MC38 tumor volume at 14 days was significantly lower (p<0.05) in the group receiving combination therapy of mROP-Survivin (200 μg/mouse) and anti-PD-1 Antibody (2 mg/kg) than in the groups receiving any other regimen. It is particularly notable that neither mROP-Survivin (200 μg) or anti-PD-1 Antibody (2 mg/kg) alone can significantly inhibit MC38 tumor growth; the mROP-Survivin:anti-PD-1 Antibody combination regimen, however, is effective in significantly reducing tumor volume.

Body Weight of Mice does not Vary with Treatment Regimen

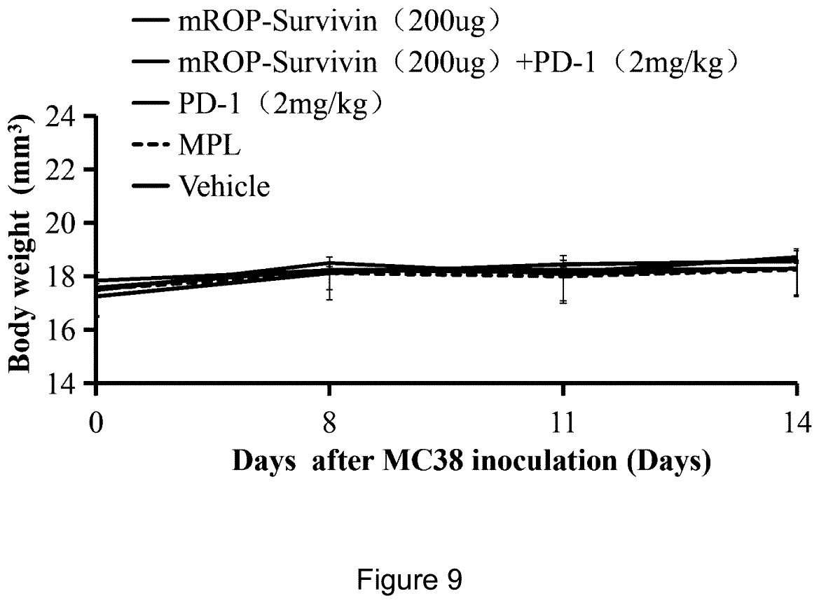

There was no significant difference in body weight observed between treatment groups at any timepoint (FIG. 9).

Combined Treatment of mROP-Survivin (200 μg/Mouse) and Anti-PD-1 Antibody (2 mg/kg) Resulted in Significantly Higher Specific T Cell Responses than in Singly Treated Groups

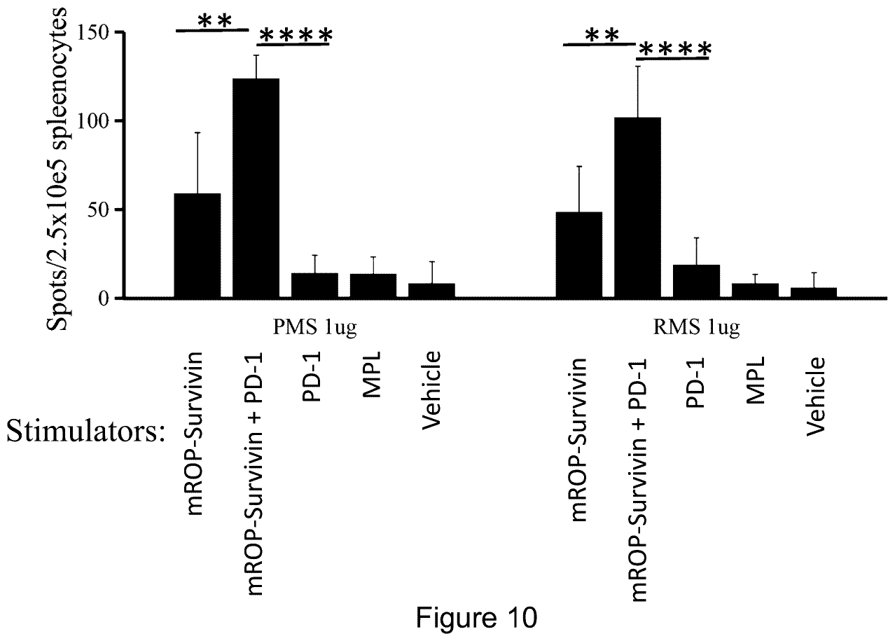

T cell responses were measured via IFN-γ release (measured by ELISPOT) from activated splenocytes harvested from the mice having received treatment as above (FIG. 10). T-cells were dramatically elevated (p<0.0001) in splenocytes having been activated by the combination of mROP-Survivin (200 μg/mouse) and anti-PD-1 Antibody (2 mg/kg) relative to control, whether stimulated with mouse survivin protein or with mouse ROP-survivin. T-cells were also significantly elevated (p<0.01) in splenocytes activated by the combination of mROP-Survivin and anti-PD-1 Antibody relative to those activated by mROP-Survivin alone, whether stimulated with PMS or with RMS. Splenocytes derived from mice receiving only anti-PD-1 Antibody did not demonstrate any T-cell response.

It can be concluded that combination treatment with mROP-Survivin and anti-PD-1 antibody is significantly more effective in vivo than either monotherapy.

### Example 4—Demonstration of a Combination Approach of a TNFR Superfamily Agonist and a Recombinant Overlapping Peptide Based on HPV-16 E7 for the Treatment of Cancer

### Materials and Methods

### Mice

A total of 40 female C57BL/6 mice were ordered from Changzhou Kavins Experimental Animal Co. Ltd. The animals were specific pathogen free and approximately 6-7 weeks old upon arrival at Changzhou Niujin Shisong Biotechnology Co., Ltd (‘CBI’). Upon receipt the animals were unpacked and placed in cages. A health inspection was performed on each animal, including evaluation of the coat, extremities and orifices. Each animal was also examined for any abnormal signs in posture or movement.

### Tumor Cell Lines

Mouse TC-1 cells were purchased from Biofeng Ltd. Mouse TC-1 expresses the E7 oncoprotein from HPV-16 and is used as a surrogate for human tumors infected with HPV-16. TC-1 cells were maintained in RPMI1640 with 10% FBS at 37° C. under 5% CO2 and subsequently cultured within 5 passages before being inoculated into the mice.

### HPV E7 Protein Synthesis

The HPV16 E7 protein gene sequence was codon optimised for expression in E. coli. The gene was synthesized by the GeneArt DNA synthesis service. The synthesized cDNA and the Bsa4 linearized vector pNIC28-Bsa4 (SGC Oxford) were treated with T4 DNA polymerase (30 min at 22° C.) in the presence of 2.5 mM dCTP and dGTP respectively. T4 DNA polymerase was inactivated by incubation at 80° C. for 20 min. T4 DNA treated PCR products and vector were mixed at a ratio of 1:50 for 10 min at 25° C. An aliquot of ligation products was used to transform DH5a competent cells. The positive clones were identified by colony PCR and the corresponding plasmids were used to transform E. coli BL21 (DE3) for protein expression.

A single transformed BL21 (DE3) colony was picked into LB (50 μg/ml Kan) and cultured overnight at 37° C. The overnight culture was then diluted 100 times with fresh LB and cultured until the OD600 reached 0.6. Then IPTG was added to final concentration of 0.5 mM. The cells were harvested at 16 hr post IPTG induction by centrifugation. The cell pellets were resuspended in lysis buffer (25 mM Tris-HCl, 200 mM NaCl, 2% Triton X-100, 10 mM imidazole, pH8.0) and were lysed by sonication. Insoluble fractions were separated by centrifugation at 20,000 g for 45 min. For expressed soluble protein, Ni-NTA resin was then added to the soluble fractions for 30 min, followed by washing with 30 resin volumes of lysis buffer and eluted with lysis buffer containing 300 mM imidazole. For proteins forming inclusion bodies, the insoluble fractions were re-suspended in buffer (25 mM Tris-HCl, 200 mM NaCl, 8 M urea, 10 mM imidazole, pH8.0) and centrifuged at 20,000 g for another 45 min, before applying to the Ni-NTA resin. Washing buffer and elution buffer also contained 8 M urea. For refolding, the eluted proteins were first buffer exchanged to 25 mM Tris-HCl, 200 mM NaCl, 0.5 arginine-HCl, pH 8.0 then to PBS using a PD10 column (GE Healthcare Life Sciences, UK).

### Tumor Models

Mice were injected in the flank subcutaneously on Day 0 with 2×10⁵ TC-1 cells mixed with RPMI 1640 medium.

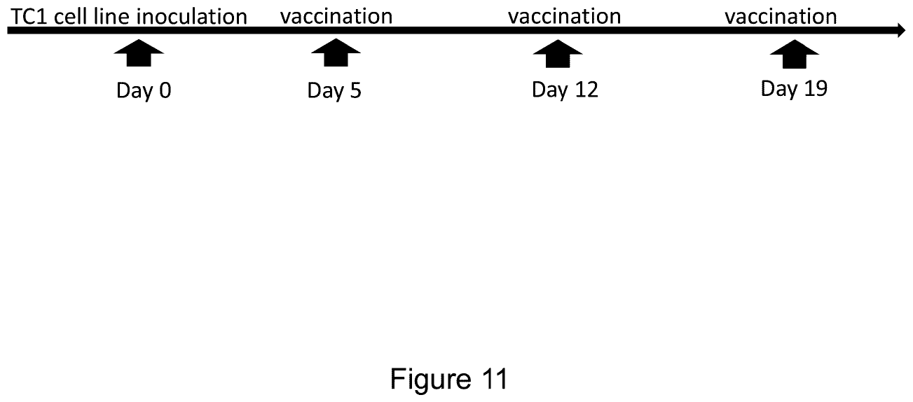

To investigate the effect of ROP-HPV16 E7 on tumor growth relative to wt HPV16 E7 protein, 40 mice were randomized into 4 groups (10 mice per group) according to body weight. Treatment was started after grouping and 5 days after cell inoculation. The groups are shown in Table 4 below. Regimen is shown in Table 4 and FIG. 11. ROP-HPV16 E7, Adjuvant (MPL), and/or PBS was administered subcutaneously every 7 days. anti-4-1BB antibody was administered intraperitoneal every 3 days.

TABLE 4

| Group | No. | Test Articles and Doses | Injection Site | Dosing Regimen |
|---|---|---|---|---|
| 1 | 10 | 100 μg ROP-HPV16 E7 + MPL | S.C. | Every 7 days |
| 2 | 10 | 100 μg Protein HPV16 E7 + MPL | S.C. | Every 7 days |
| 3 | 10 | Adjuvant (MPL) | S.C. | Every 7 days |
| 4 | 10 | PBS | S.C. | Every 7 days |

(S.C. = subcutaneous; i.p. = intraperitoneal)

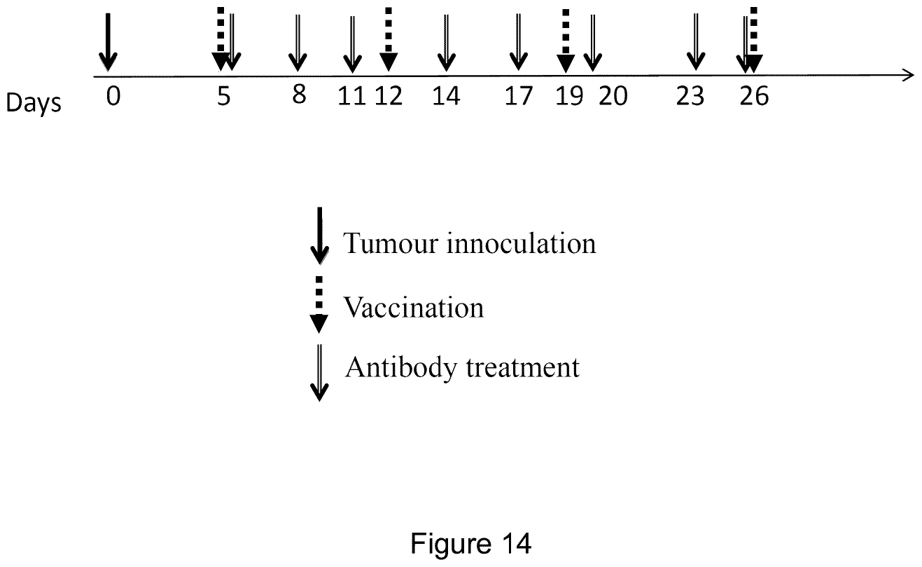

To assess a combination therapy of ROP-HPV16 E7 and anti-4-1BB antibody, 50 mice were randomized into 5 groups (10 mice per group) according to body weight five days after tumor cell inoculation. Treatment was started after grouping and five days after cell inoculation. The groups are shown in Table 5 below. Regimen is shown in Table 5 and FIG. 14. ROP-HPV16 E7, Adjuvant (MPL), and PBS were administered subcutaneously every 7 days. anti-4-1BB antibody was administered intraperitoneal every 3 days. Adjuvant was used in groups 1-4.

TABLE 5

| | | | Injection | Dosing |
|---|---|---|---|---|
| Group | No. | Test Articles and Doses | Site | Regimen |
| 1 | 10 | 100 μg ROP-HPV16 E7 + | S.C. | Every 7 days |
| | | 4-1BB Ab (10 mg/kg) | i.p. | Every 3 days |
| 2 | 10 | 100 μg ROP-HPV16 E7 | S.C. | Every 7 days |
| 3 | 10 | Adjuvant (MPL) | S.C. | Every 7 days |
| 4 | 10 | 4-1BBAb (10 mg/kg) | i.p. | Every 3 days |
| 5 | 10 | PBS | S.C. | Every 7 days |

(S.C. = subcutaneous; i.p. = intraperitoneal)

ROP-HPV16 E7 was produced in accordance with Example 1. Monophosphoryl Lipid A adjuvant (MPL) was purchased from Sigma Aldrich, Dorset, UK (catalogue number S6322). The agonist 4-1BB (CD137) antibody (BE0239) was purchased from BioXCell (New Hampshire, USA). The MPL as provided contains 0.5 mg of MPL, and this was diluted into 2 ml of PBS or protein solution such that the concentration of MPL was 0.25 mg·ml⁻¹.

Tumors were measured every 3 days (length×width) with a caliper. Tumor volume was determined using the formula: ½×D×d² where D is the major axis and d is the minor axis. Mice were sacrificed when tumors reached 2 cm³ or upon ulceration.

Mice were sacrificed when tumors reached 2 cm³ or upon ulceration.

### Statistical Analysis

Results were expressed as mean±S.E.M. Comparisons were made by Student's t-test or logrank test as appropriate, p<0.05 was considered to be significant.

### Results

### ROP-HPV16 E7inhibited Tumor Growth.

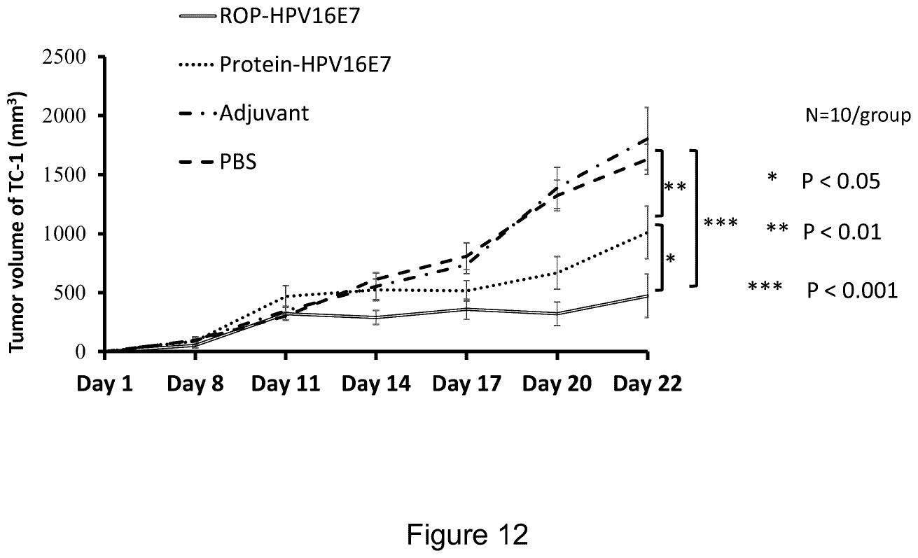

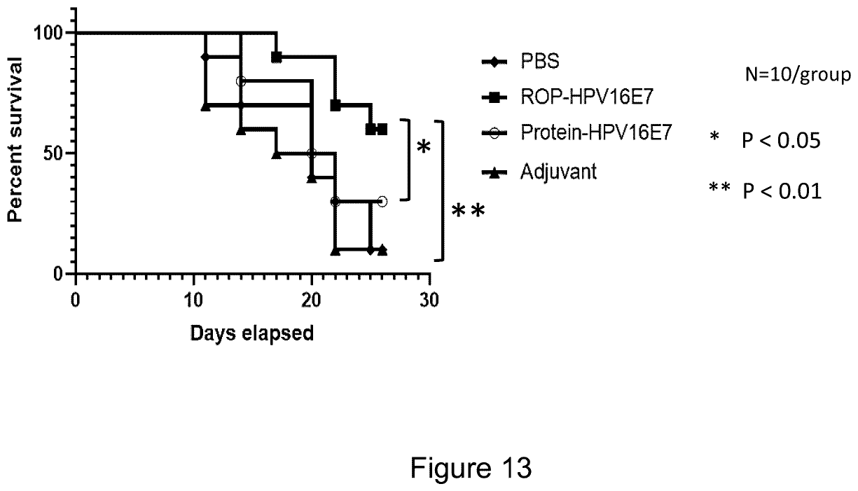

TC-1-inoculated mice receiving wt protein HPV16 E7 showed tumor volumes two thirds that of controls at 22 Days (p<0.01). However, mice receiving ROP-HPV16 E7 showed even greater inhibition of tumor growth, having at 22 Days tumor volume less than one third that of controls (p<0.001) and half that of mice treated with wt protein (p<0.05) (FIG. 12). This benefit translated into survival outcomes. Mice receiving ROP-HPV16 E7 likewise demonstrated best survival outcome (FIG. 13): 60% mice receiving ROP-HPV16 E7 survived to Day 26, significantly higher (p<0.01) than the survival rate of mice in control groups (10%) (adjuvant or PBS) and significantly (p<0.05) higher than the survival rate of mice treated with wt HPV16 E7 (30%).

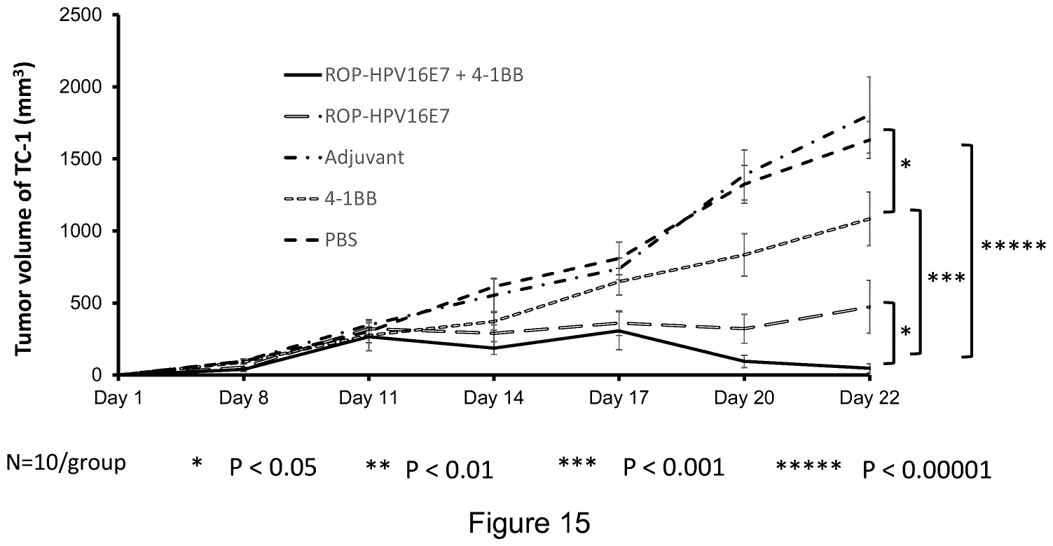

ROP-HPV16 E7 demonstrated potent synergy in combination with anti-4-1BB antibody, leading to tumor regression and improved survival Treatment with ROP-HPV16 E7 alone or anti-4-1BB Ab alone slows tumor growth, with ROP-HPV16 E7 slowing tumor growth to a greater extent than anti-4-1BB Ab (FIG. 15). However, while these treatments slow tumor growth, treatment of TC-1-inoculated mice with a combination of ROP-HPV16 E7 (100 μg) and anti-4-1BB antibody (10 mg/kg) results in tumor regression to near-zero levels at Day 22 (FIG. 15).Tumor volumes of mice treated with combination therapy are significantly lower at Day 22 than tumor volume of mice treated with any other regimen: control (p<0.00001), 4-1BB alone (p<0.001), or ROP-HPV16 E7 alone (p<0.05).

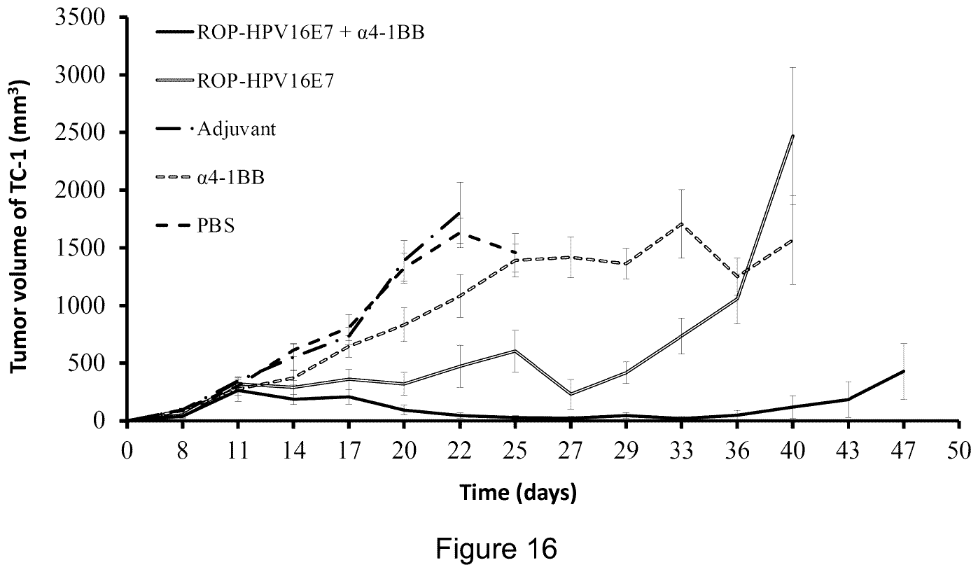

Mice with tumour size more than 1500 mm³ are culled. Data ceases at 22 days for Adjuvant and 25 Days for PBS due to 0% survival (FIG. 16). When treatment is continued past 22 days, tumor volume of mice treated with anti-4-1BB Ab is seen to plateau, capped at 1500 mm³ (data stops at Day 40 due to 0% survival) (FIG. 16). Tumor volume of mice treated with ROP-HPV16 E7 alone increases rapidly from below 500 to levels equivalent to anti-4-1EE Ab or higher. Mice treated with combination therapy, however, exhibit very low (near-zero) tumor volume until day 40 (when a slight uptick in growth begins to be seen) (FIG. 16), demonstrating potent anti-tumor activity. This anti-tumor activity is more pronounced than would be expected through merely additive effects of ROP-HPV16 E7 and anti-4-1BB Ab; i.e. synergy is demonstrated. Mice treated with combination therapy remain tumour free over a follow-up period of 140 days (data not shown).

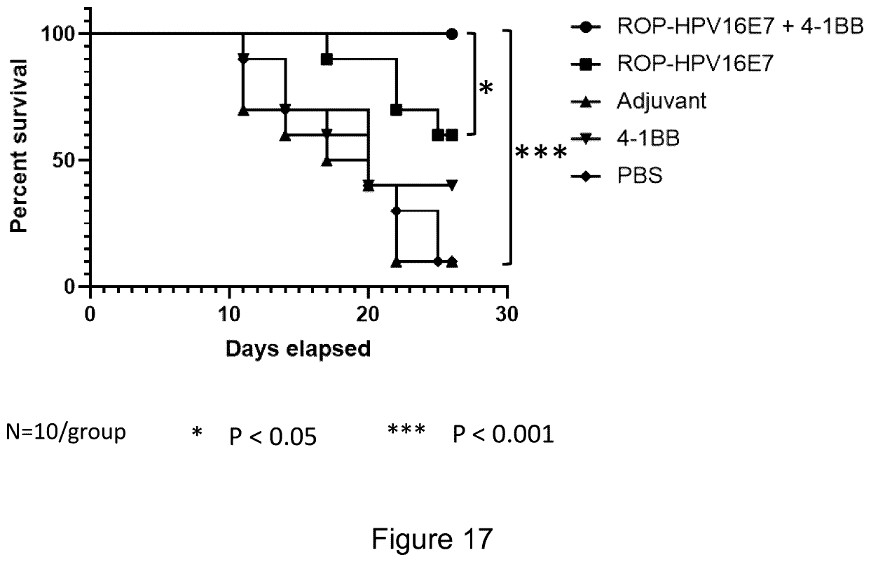

FIGS. 17 & 18 show that survival rate of the combination treatment group is far higher than all other groups (ROP-HPV16 E7⁺ anti-4-1BB Ab vs ROP-HPV16 E7, p<0.05 at Day 26; ROP-HPV16 E7⁺ anti-4-1BB Ab vs anti-4-1BB Ab, p<0.001 at Day 26; ROP-HPV16 E7+anti-4-1BB Ab vs PBS, p<0.001 at Day 26). 100% mice in the combination group survived to Day 47, while survival of the ROP-HPV16 E7 group fell to 30% over the course of treatment, and survival in anti-4-1BB Ab group and control groups to 0%.

In conclusion, a combination treatment of ROP-HPV16 E7 and anti-4-1BB antibody is highly effective in vivo.

### Example 5—Demonstration of a Combination Approach of a Checkpoint Inhibitor and a Recombinant Overlapping Peptide Based on HPV-16 E7 for the Treatment of Cancer

### Materials and Methods

### Mice

A total of 50 female C57BL/6 mice are ordered from Changzhou Kavins Experimental Animal Co. LTD. The animals are specific pathogen free and approximately 6-7 weeks old upon arrival at CBI. Upon receipt the animals are unpacked and placed in cages. A health inspection is performed on each animal, including evaluation of the coat, extremities and orifices. Each animal is also examined for any abnormal signs in posture or movement.

### Tumor Cell Lines

Mouse TC-1 cells were purchased from Biofeng Ltd. Cells were maintained at 37° C. under 5% CO2 in RPM11640 medium supplemented with 10% FBS and subsequently cultured within 5 passages before inoculated into the mice.

### Tumor Models

Mice were injected in the flank subcutaneously on Day 0 with 2×10⁵ TC-1 cells resuspended in serum free RPMI 1640 medium.

To assess a combination therapy of ROP-HPV16 E7 and anti-PD-1 blocking antibody, 50 mice were randomized into 5 groups (10 mice per group) according to body weight five days after tumor cell inoculation. Treatment was started after grouping and five days after cell inoculation. The groups are shown in Table 6 below. ROP-HPV16 E7, optionally also PD-1 Ab, was delivered with MPL. Regimen is shown in Table 6 and FIG. 14. ROP-HPV16 E7, Adjuvant (MPL), and PBS were administered subcutaneously every 7 days. anti-PD-1 antibody was administered intraperitoneal every 3 days.

TABLE 6

| | | | Injection | Dosing |
|---|---|---|---|---|
| Group | No. | Test Articles and Doses | Site | Regimen |
| 1 | 10 | 100 μg ROP-HPV16 E7 + | S.C. | Every 7 days |
| | | PD-1 Ab (10 mg/kg) | i.p. | Every 3 days |
| 2 | 10 | 100 μg ROP-HPV16 E7 | S.C. | Every 7 days |
| 3 | 10 | Adjuvant | S.C. | Every 7 days |
| 4 | 10 | PD-1Ab (10 mg/kg) | i.p. | Every 3 days |
| 5 | 10 | PBS | S.C. | Every 7 days |

(S.C. = subcutaneous; i.p. = intraperitoneal)

Tumors are measured every 3 days (length×width) with a caliper. Tumor volume is determined using the formula: ½×D×d² where D is the major axis and d is the minor axis. Mice are sacrificed when tumors reached 2 cm³ or upon ulceration.

Mice were sacrificed when tumors reached 2 cm³ or upon ulceration.

### Statistical Analysis

Results are expressed as mean±S.E.M. Comparisons are made by Student's t-test or logrank test as appropriate, p<0.05 is considered to be significant.

### Results

Combined Treatment of ROP-HPV16 E7 (100 μg/Mouse) and Anti-PD-1 Antibody (10 mg/kg) Significantly Reduced Growth of TC-1 Tumor in Mice

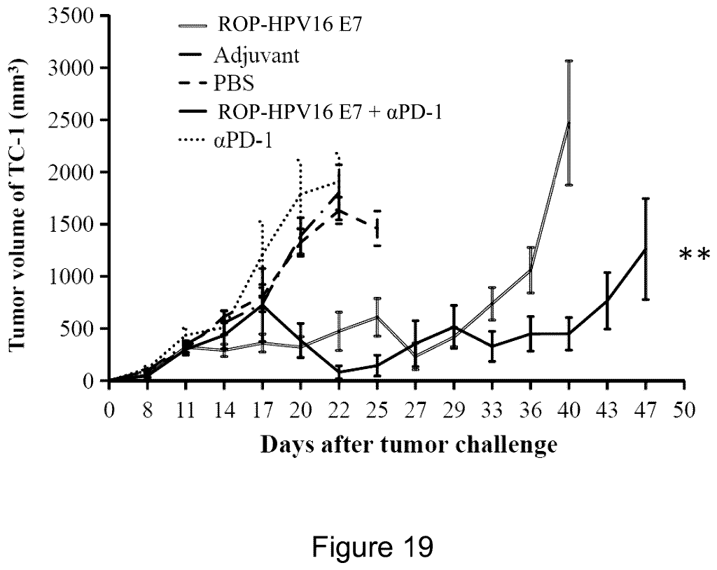

As can be seen from FIG. 19, tumor volume increases rapidly in mice receiving treatment with adjuvant, PBS, and αPD-1 antibody up to approximately 1500-2000 mm³ within 20-22 days. Tumors in mice receiving treatment with ROP-HPV16 E7 alone remain of low (approximately 500 mm³) until day 33 when volume rapidly increases. Tumors in mice receiving combination treatment, however, remain low (approximately 500 mm³) until days 40-43. Combination therapy therefore provided the most effective treatment option. The treatment with ROP-HPV16 E7 showed a statistically significant reduction in tumour volume compared to the other treatment groups over the treatment period (p<0.01).

Combined Treatment of ROP-HPV16 E7 (100 μg/Mouse) and Anti-PD-1 Antibody (10 mq/kg) Resulted in Significantly Higher Survival than in Singly Treated Groups

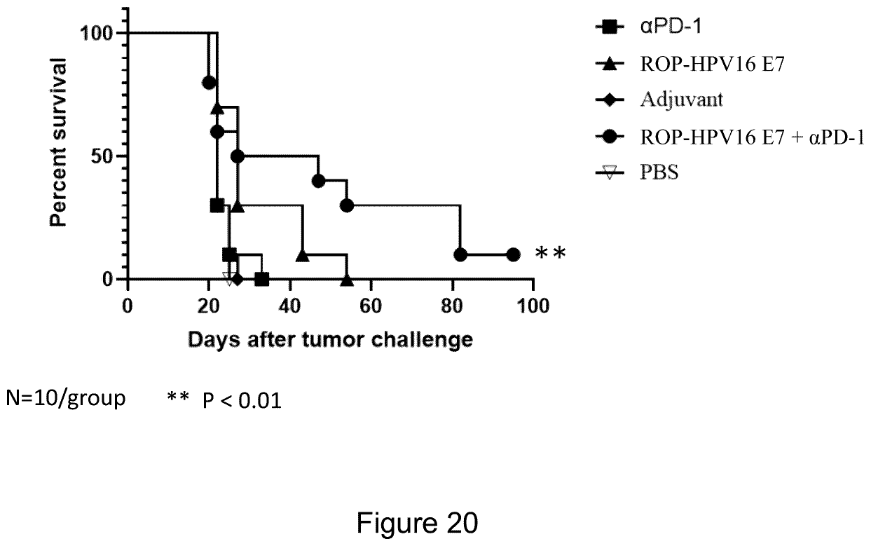

FIG. 20 demonstrates that survival rates are highest in mice receiving a combination treatment of ROP-HPV16 E7 (100 μg/mouse) and αPD-1 antibody (10 mg/kg), with 10% mice surviving to end of the trial. By contrast, survival of the group receiving ROP-HPV16 E7 alone was 30% at Day 47, and 0% survival at Day 47 in remaining groups receiving αPD-1 antibody alone, PBS, or adjuvant. Treatment with ROP-HPV16 E7 showed a statistically significant increase in survival in mice compared to the other treatment groups over the measurement period (p<0.01).

It can be concluded that combination of ROP-HPV-E7 vaccination with anti-PD-1 treatment results in synergetic tumour inhibition effect.

Together, the Examples demonstrate the ability of polypeptides of the invention to lower the effective dose of TNFR Superfamily receptor agonists, such as 4-1 BB, and/or to increase their maximal efficacy. Similarly, they demonstrate the ability of polypeptides of the invention to increase the potency and/or efficacy of checkpoint inhibitors such as anti-PD-1. Unexpectedly, effects of a combination therapy of a polypeptide of the invention and an immuno-oncology agent—for example a TNFR Superfamily receptor agonist or a checkpoint inhibitor—are synergistic. There exists in the art a need to improve the potency and efficacy of existing anti-cancer immunotherapies to improve safety, tolerability, and clinical outcomes; various approaches have been attempted without appreciable success. There are numerous barriers to success: one is the difficultly of identifying efficacious combination regimes from the sheer number of immunotherapies contemplatable, as is apparent from the vast volume of related academic literature. One such approach speculatively contemplated, but not realized, is a combination regimen of a multi-peptide construct with an immune-oncology agent (Oxford Vacmedix Ltd, Biopharmerdealmaker Advertisement Feature, 2017). Another barrier to success is the difficulty of achieving reliable and consistent therapeutic responses across different patients and cancer types and stages (Ventola, 2017). The present invention is based upon the unexpected finding that a combination of a polypeptide of the present invention with a TNFRSF agonist or with a checkpoint inhibitor augments anti-cancer activity of both polypeptide and agent synergistically. Synergy takes the form of synergistic potency, synergistic efficacy, or both. A polypeptide of the invention derived from and immunologically targeted to survivin is unexpectedly able to increase the potency of 4-1BB agonist to allow administration at a non-toxic dose (FIG. 2) and to increase the efficacy of anti-PD-1 (FIG. 8) particularly via significantly elevated T cell responses (FIG. 10). A polypeptide of the invention derived from and immunologically targeted to HPV16 E7 unexpectedly demonstrates synergy with 4-1BB agonist, leading to significant tumour regression (FIG. 15) and 100% survival rates in mice across all timepoints measured (FIGS. 17 & 18). Because the polypeptide of the invention comprises multiple peptide fragments, optionally which cover whole tumour antigen proteins or immunologically relevant portions thereof, further optionally in an overlapping fashion, the polypeptides are applicable across broad HLA-types without the need for HLA-typing or patient screening before treatment. The skilled person would appreciate that polypeptides of the present invention may be readily targeted to cancer- or even patient-specific markers, thereby further broadening the patient base who may benefit from treatment with the immunotherapy delivered in combination.

### REFERENCES

Cai, L., Zhang, J., Zhu, R., Shi, W., Xia, X., Edwards, M., Finch, W., Coombs, A., Gao, J., Chen, K., Owen, S., Jiang, S., & Lu, W. Protective cellular immunity generated by cross-presenting recombinant overlapping peptide proteins. Oncotarget vol. 8, 44 76516-76524, (2017).
Compte M, Harwood S L, Munoz I G, Navarro R, Zonca M, Perez-Chacon G, Erce-Llamazares A, Merino N, Tapia-Galisteo A, Cuesta A M, Mikkelsen K, Caleiras E, Nunez-Prado N, Aznar M A, Lykkemark S, Martinez-Torrecuadrada J, Melero I, Blanco F J, Bernardino de la Serna J, Zapata J M, Sanz L, Alvarez-Vallina L. A tumor-targeted trimeric 4-1 BB-agonistic antibody induces potent anti-tumor immunity without systemic toxicity. Nat Commun 9, 4809 (2018).
Gong J, Chehrazi-Raffle A, Reddi S, Salgia R. Development of PD-1 and PD-L1 inhibitors as a form of cancer immunotherapy: a comprehensive review of registration trials and future considerations. Journal for Immuno Therapy of Cancer 6:8 (2018).
Kallinteris N L, Wu S, Lu X, Humphreys R E, von Hofe E, Xu M. Enhanced CD4+ T-cell response in DR4-transgenic mice to a hybrid peptide linking the li-Key segment of the invariant chain to the melanoma gp100(48-58) MHC class II epitope. J Immunother. 28(4) 352-358, (2005).
Oxford Vacmedix Ltd.: harnessing the power of immunotherapy to treat and monitor cancer, Profile: Biopharmerdealmaker Advertisement Feature, September 2017, https://www.nature.com/articles/d43747-020-00345-4
Qi X, Li F, Wu Y, Cheng C, Han P, Wang J, Yang X. Optimization of 4-1 BB antibody for cancer immunotherapy by balancing agonistic strength with FcγR affinity. Nat Commun 10, 2141 (2019).
Segal N H, Logan T F, Hodi F S, McDermott D, Melero I, Hamid O, Schmidt H, Robert C, Chiarion-Sileni V, Ascierto P A, Maio M, Urba W J, Gangadhar T C, Suryawanshi S, Neely J, Jure-Kunkel M, Krishnan S, Kohrt H, Sznol M, Levy R. Results from an Integrated Safety Analysis of Urelumab, an Agonist Anti-CD137 Monoclonal Antibody. Clin Cancer Res 23:8 1929-1936 (2017).
Spiers, L., Coupe, N., & Payne, M. Toxicities associated with checkpoint inhibitors—an overview. Rheumatology (Oxford, England) vol. 58,Suppl 7 (2019).
Timmerman, J., Herbaux, C., Ribrag, V., Zelenetx, A. D., Houot, R., Neelapu, S. S., Logan, T., Lossos, I. S., Urba, W., Salles, G., Ramchandren, R., Jacobson, C., Godwin, J, Carpio, C., Lathers, D., Liu, Y., Neely, J., Suryawanshi, S., Koguchi, Y., Levy, R. Urelumab alone or in combination with rituximab in patients with relapsed or refractory B-cell lymphoma, Am J Hematol 95:5 510-520 (2020)
Ventola C L. Cancer Immunotherapy, Part 3: Challenges and Future Trends. P T. 2017; 42(8):514-521.
Waldman, A. D., Fritz, J. M. & Lenardo, M. J. A guide to cancer immunotherapy: from T cell basic science to clinical practice. Nat Rev Immunol (2020).
Wheatley, S. P. & Altieri, D.C. Survivin at a Glance. Journal of Cell Science 132:7 (2019).
Xu M, Lu X, Sposato M, Zinckgraf J W, Wu S, von Hofe E. li-Key/HPV16 E7 hybrid peptide immunotherapy for HPV16+cancers. Vaccine 27(34) 4641-4647, (2009).
Zhang H, Hong H, Li D, Ma S, Di Y, Stoten A, Haig N, Di Gleria K, Yu Z, Xu X N, McMichael A, Jiang S. Comparing pooled peptides with intact protein for accessing cross-presentation pathways for protective CD8+ and CD4+ T cells. The Journal of Biological Chemistry. 284(14) 9184-9191, (2009).

1. A method for the treatment of cancer in a subject comprising:
administering, to the subject, a polypeptide comprising two or more peptide fragments, wherein a first peptide fragment comprises a first sequence derived from a first tumour antigen protein and wherein a second peptide fragment comprises a second sequence derived from a second tumour antigen protein, further comprising one or more exogenous cathepsin cleavage site sequences located between each of the two or more peptide fragments; and
administering, to the subject, an immuno-oncology agent.

2. The method of claim 1, wherein the first tumour antigen and/or the second tumour antigen protein is a tumour specific antigen, a tumour associated antigen, or a cancer/testis antigen.

3. The method of claim 1 or 2, wherein the first tumour antigen protein and the second tumour antigen protein are the same tumour antigen protein.

4. The method of any one of the preceding claims, wherein the first tumour antigen protein and/or second tumour antigen protein is a self-antigen, an altered-self-antigen, or a non-self-antigen.

5. The method of any one of the preceding claims, wherein the tumour antigen protein is survivin.

6. The method of any one of claims 1 to 4, wherein the tumour antigen protein is a viral-derived cancer antigen, optionally an HPV protein, further optionally an HPV16 protein.

7. The method of claim 6, wherein the tumour antigen protein is HPV16 E7.

8. The method of any one of the preceding claims, wherein the one or more exogenous cathepsin cleavage site sequences is a cathepsin S cleavage sequence, preferably an LRMK cleavage sequence.

9. The method of any one of the preceding claims, wherein the polypeptide and the immuno-oncology agent are administered to the subject simultaneously, separately, or sequentially.

10. The method of any one of the preceding claims, wherein the immuno-oncology agent is a TNFR Superfamily agonist, or a checkpoint inhibitor.

11. The method of any one of the preceding claims, wherein each administration of the polypeptide comprises between 1 μg·kg⁻¹ to 2000 μg·kg⁻¹ of the polypeptide, preferably 5 to 20 μg·kg⁻¹ or lower.

12. The method of claim 10 or 11, wherein the TNFR Superfamily agonist is a peptide or fragment thereof, a glycoprotein or fragment thereof, or a small molecule, or wherein the checkpoint inhibitor is a peptide or fragment thereof, a glycoprotein or fragment thereof, or a small molecule.

13. The method of any one of claims 10 to 11, wherein the TNFR Superfamily agonist is an antibody, or fragment thereof, or the checkpoint inhibitor is an antibody, or fragment thereof.

14. The method of any one of claims 10 to 13, wherein the TNFR Superfamily agonist is administered at a dose non-toxic to humans.

15. The method of any one of claims 10 to 14, wherein the TNFR Superfamily agonist is a 4-1BB agonist, or wherein the checkpoint inhibitor is a PD-1 antagonist.

16. The method of claim 15, wherein the 4-1BB agonist is administered at a dose below 1 mg·kg¹.

17. The method of any one of the preceding claims, wherein the administration of the polypeptide and the immuno-oncology agent to the subject is repeated periodically, preferably every 3, 4, 5, 6, or 7 days.

18. The method of any one of the preceding claims, wherein the two or more peptide fragments comprise one or more overlapping sequences.

19. The method of claim 18, wherein the one or more overlapping sequences are between 2 and 31 amino acids in length, optionally wherein the one or more overlapping sequences are at least 8 amino acids in length.

20. The method of any one of the preceding claims, wherein the polypeptide is delivered in a delivery vehicle, optionally further comprising administering the delivery vehicle comprising the polypeptide or the polypeptide in a pharmaceutically acceptable carrier.

21. A composition for use in the treatment of cancer, wherein the composition comprises a polypeptide comprising two or more peptide fragments, wherein a first peptide fragment comprises a first sequence derived from a tumour antigen protein and wherein a second peptide fragment comprises a second sequence derived from a tumour antigen protein, further comprising one or more exogenous cathepsin cleavage site sequences located between each of the two or more peptide fragments,
and wherein the treatment comprises co-administration of the polypeptide with an immuno-oncology agent.

22. A composition for use according to claim 21, further comprising the polypeptide as described in any one of claims 1 to 20, and/or the method as described in any one of claims 1 to 20.

23. A method of determining whether a cancer is suitable for treatment according to the method of any one of claims 1 to 20, comprising:
administering to a subject or an in vitro sample a polypeptide comprising two or more peptide fragments, wherein a first peptide fragment comprises a first sequence derived from a tumour antigen protein and wherein a second peptide fragment comprises a second sequence derived from a tumour antigen protein, further comprising one or more exogenous cathepsin cleavage site sequences located between each of the two or more peptide fragments;
administering to a subject or an in vitro sample an immuno-oncology agent; and
measuring T cell stimulation in said subject or in vitro sample.

24. An immuno-oncology agent for use in the treatment of cancer, wherein the treatment comprises administering the immuno-oncology agent and a polypeptide comprising two or more peptide fragments, wherein a first peptide fragment comprises a first sequence derived from a tumour antigen protein and wherein a second peptide fragment comprises a second sequence derived from a tumour antigen protein, further comprising one or more exogenous cathepsin cleavage site sequences located between each of the two or more peptide fragments.

25. An immuno-oncology agent for use according to claim 24, wherein the co-administered polypeptide is a polypeptide as described in any one of claims 1 to 20, and/or wherein the treatment of cancer is by the method as described in any one of claims 1 to 20.

26. A kit for the treatment of cancer comprising:
a polypeptide comprising two or more peptide fragments, wherein the first peptide fragment comprises a first sequence derived from a tumour antigen protein and wherein the second peptide fragment comprises a second sequence derived from a tumour antigen protein, further comprising one or more exogenous cathepsin cleavage site sequences located between each of the two or more peptide fragments, and an immuno-oncology agent.

27. The kit of claim 26, wherein the immuno-oncology agent is
a TNFR Superfamily agonist, optionally wherein the TNFR Superfamily agonist is a peptide or fragment thereof, a glycoprotein or fragment thereof, a small molecule, or an antibody or fragment thereof, or
a checkpoint inhibitor, optionally wherein the checkpoint inhibitor is a peptide or fragment thereof, a glycoprotein or fragment thereof, a small molecule, or an antibody or fragment thereof.

28. The kit of claim 27, further comprising one or more pharmaceutically acceptable carriers or a nucleic acid encoding the polypeptide.

29. The kit of claim 27 or 28, wherein the TNFR Superfamily agonist is a 4-1 BB agonist, or wherein the checkpoint inhibitor is a PD-1 antagonist.


SEQUENCE LISTING (52 sequences)

SEQ ID NO: 1 - protein, 142 aa, Homo sapiens
MGAPTLPPAWQPFLKDHRISTFKNWPFLEGCACTPERMAEAGFIHCPTENEPDLAQCFFCFKELEGWEPDDDPIEEHKKHSSGCAFLSVKKQFEELTLGEFLKLDRERAKNKIAKETNNKKKEFEETAKKVRRAIEQLAAMD

SEQ ID NO: 2 - protein, 165 aa, Homo sapiens
MGAPTLPPAWQPFLKDHRISTFKNWPFLEGCACTPERMAEAGFIHCPTENEPDLAQCFFCFKELEGWEPDDDPIGPGTVAYACNTSTLGGRGGRITREEHKKHSSGCAFLSVKKQFEELTLGEFLKLDRERAKNKIAKETNNKKKEFEETAKKVRRAIEQLAAMD

SEQ ID NO: 3 - protein, 137 aa, Homo sapiens
MGAPTLPPAWQPFLKDHRISTFKNWPFLEGCACTPERMAEAGFIHCPTENEPDLAQCFFCFKELEGWEPDDDPMQRKPTIRRKNLRKLRRKCAVPSSSWLPWIEASGRSCLVPEWLHHFQGLFPGATSLPVGPLAMS

SEQ ID NO: 4 - protein, 120 aa, Homo sapiens
MGAPTLPPAWQPFLKDHRISTFKNWPFLEGCACTPERMAEAGFIHCPTENEPDLAQCFFCFKELEGWEPDDDPIEEHKKHSSGCAFLSVKKQFEELTLGEFLKLDRERAKNKIERALLAE

SEQ ID NO: 5 - protein, 117 aa, Homo sapiens
MGAPTLPPAWQPFLKDHRISTFKNWPFLEGCACTPERMAEAGFIHCPTENEPDLAQCFFCFKELEGWEPDDDPIEEHKKHSSGCAFLSVKKQFEELTLGEFLKLVRETLPPPRSFIR

SEQ ID NO: 6 - protein, 78 aa, Homo sapiens
MGAPTLPPAWQPFLKDHRISTFKNWPFLEGCACTPERMAEAGFIHCPTENEPDLAQCFFCFKELEGWEPDDDPMRELC

SEQ ID NO: 7 - protein, 74 aa, Homo sapiens
MGAPTLPPAWQPFLKDHRISTFKNWPFLEGCACTPERMAEAGFIHCPTENEPDLAQCFFCFKELEGWEPDDDPM

SEQ ID NO: 8 - protein, 30 aa, Homo sapiens
MGAPTLPPAWQPFLKDHRISTFKNWPFLEG

SEQ ID NO: 9 - protein, 30 aa, Homo sapiens
DHRISTFKNWPFLEGCACTPERMAEAGFIH

SEQ ID NO: 10 - protein, 28 aa, Homo sapiens
ACTPERMAEAGFIHCPTENEPDLAQCFF

SEQ ID NO: 11 - protein, 29 aa, Homo sapiens
PTENEPDLAQCFFCFKELEGWEPDDDPIE

SEQ ID NO: 12 - protein, 30 aa, Homo sapiens
FKELEGWEPDDDPIEEHKKHSSGCAFLSVK

SEQ ID NO: 13 - protein, 28 aa, Homo sapiens
EHKKHSSGCAFLSVKKQFEELTLGEFLK

SEQ ID NO: 14 - protein, 29 aa, Homo sapiens
QFEELTLGEFLKLDRERAKNKIAKETNNK

SEQ ID NO: 15 - protein, 30 aa, Homo sapiens
RERAKNKIAKETNNKKKEFEETAEKVRRAI

SEQ ID NO: 16 - protein, 21 aa, Homo sapiens
KEFEETAEKVRRAIEQLAAMD

SEQ ID NO: 17 - protein, 4 aa, Artificial Sequence
  A cleavage site for cathepsin S
LRMK

SEQ ID NO: 18 - protein, 287 aa, Artificial Sequence
  An artificial peptide with the peptide fragments derived from human survivin isoform 1.
MGAPTLPPAWQPFLKDHRISTFKNWPFLEGLRMKDHRISTFKNWPFLEGCACTPERMAEAGFIHLRMKACTPERMAEAGFIHCPTENEPDLAQCFFLRMKPTENEPDLAQCFFCFKELEGWEPDDDPIELRMKFKELEGWEPDDDPIEEHKKHSSGCAFLSVKLRMKEHKKHSSGCAFLSVKKQFEELTLGEFLKLRMKQFEELTLGEFLKLDRERAKNKIAKETNNKLRMKRERAKNKIAKETNNKKKEFEETAEKVRRAILRMKKEFEETAEKVRRAIEQLAAMD

SEQ ID NO: 19 - DNA, 879 bp, Artificial Sequence
  A polynucleotide encoding an artificial protein derived from human survivin isoform 1.
ggatcccatatgggtgcaccaactcttcctccagcatggcaacctttcctgaaggatcatcgtatctctactttcaagaactggccattcctggaaggtctgcgtatgaaggatcaccgtatctctactttcaagaactggccattccttgagggttgtgcttgtactcctgagcgtatggctgaggctggtttcatccacctgcgtatgaaggcttgcactcctgaacgtatggctgaagctggtttcatccactgtccaactgagaacgagcctgatctggcacaatgcttcttccttcgtatgaagcctactgagaacgaacctgatctggctcagtgcttcttctgcttcaaggaacttgagggttgggagcctgatgatgatccaatcgagctgcgtatgaagttcaaggagctggaaggttgggagcctgatgatgatcctatcgaggagcacaagaagcgctcttctggttgtgctttcctgtctgtcaaactgcgtatgaaggagcacaagaagcactcttctggttgtgctttcctgtctgtcaagaagcagttcgaagaactgactctgggtgagttcctgaagctgcgtatgaagcagttcgaggagctgactctgggtgagttcctgaagctggatcgtgaacgtgctaagaacaagatcgctaaggagactaacaacaagctgcgtatgaagcgtgagcgtgctaagaacaagatcgctaaggagactaacaacaagaagaaggagttcgaggagactgctgagaaggttcgtcgtgctatccttcgtatgaagaaggagttcgaggagactgctgagaaggttcgtcgtgctatcgagcagctggctgccatggactaactcgag

SEQ ID NO: 20 - DNA, 426 bp, Homo sapiens
atgggcgcccccaccctgccccccgcctggcagcccttcctgaaggaccacaggatcagcaccttcaagaactggcccttcctggagggctgcgcctgcacccccgagaggatggccgaggccggcttcatccactgccccaccgagaacgagcccgacctggcccagtgcttcttctgcttcaaggagctggagggctgggagcccgacgacgaccccatcgaggagcacaagaagcacagcagcggctgcgccttcctgagcgtgaagaagcagttcgaggagctgaccctgggcgagttcctgaagctggacagggagagggccaagaacaagatcgccaaggagaccaacaacaagaagaaggagttcgaggagaccgccaagaaggtgaggagggccatcgagcagctggccgccatggac

SEQ ID NO: 21 - DNA, 60 bp, Artificial Sequence
  A polynucleotide encoding part of a polypeptide of the present invention (number 1 of 22 synthetic gene fragments).
ggatcccatatgggtgcaccaactcttcctccagcatggcaacctttcctgaaggatcat

SEQ ID NO: 22 - DNA, 60 bp, Artificial Sequence
  A polynucleotide encoding part of a polypeptide of the present invention (number 2 of 22 synthetic gene fragments).
gaccttccaggaatggccagttcttgaaagtagagatacgatgatccttcaggaaaggtt

SEQ ID NO: 23 - DNA, 60 bp, Artificial Sequence
  A polynucleotide encoding part of a polypeptide of the present invention (number 3 of 22 synthetic gene fragments).
ctggccattcctggaaggtctgcgtatgaaggatcaccgtatctctactttcaagaactg

SEQ ID NO: 24 - DNA, 60 bp, Artificial Sequence
  A polynucleotide encoding part of a polypeptide of the present invention (number 4 of 22 synthetic gene fragments).
catacgctcaggagtacaagcacaaccctcaaggaatggccagttcttgaaagtagagat

SEQ ID NO: 25 - DNA, 60 bp, Artificial Sequence
  A polynucleotide encoding part of a polypeptide of the present invention (number 5 of 22 synthetic gene fragments).
cttgtactcctgagcgtatggctgaggctggtttcatccacctgcgtatgaaggcttgca

SEQ ID NO: 26 - DNA, 60 bp, Artificial Sequence
  A polynucleotide encoding part of a polypeptide of the present invention (number 6 of 22 synthetic gene fragments).
ggacagtggatgaaaccagcttcagccatacgttcaggagtgcaagccttcatacgcagg

SEQ ID NO: 27 - DNA, 60 bp, Artificial Sequence
  A polynucleotide encoding part of a polypeptide of the present invention (number 7 of 22 synthetic gene fragments).
gctggtttcatccactgtccaactgagaacgagcctgatctggcacaatgcttcttcctt

SEQ ID NO: 28 - DNA, 60 bp, Artificial Sequence
  A polynucleotide encoding part of a polypeptide of the present invention (number 8 of 22 synthetic gene fragments).
actgagccagatcaggttcgttctcagtaggcttcatacgaaggaagaagcattgtgcca

SEQ ID NO: 29 - DNA, 60 bp, Artificial Sequence
  A polynucleotide encoding part of a polypeptide of the present invention (number 9 of 22 synthetic gene fragments).
cgaacctgatctggctcagtgcttcttctgcttcaaggaacttgagggttgggagcctga

SEQ ID NO: 30 - DNA, 60 bp, Artificial Sequence
  A polynucleotide encoding part of a polypeptide of the present invention (number 10 of 22 synthetic gene fragments).
cagctccttgaacttcatacgcagctcgattggatcatcatcaggctcccaaccctcaag

SEQ ID NO: 31 - DNA, 60 bp, Artificial Sequence
  A polynucleotide encoding a part of a polypeptide of the present invention (number 11 of 22 synthetic gene fragments).
gtatgaagttcaaggagctggaaggttgggagcctgatgatgatcctatcgaggagcaca

SEQ ID NO: 32 - DNA, 60 bp, Artificial Sequence
  A polynucleotide encoding a part of a polypeptide of the present invention (number 12 of 22 synthetic gene fragments).
agtttgacagacaggaaagcacaaccagaagagtgcttcttgtgctcctcgataggatca

SEQ ID NO: 33 - DNA, 60 bp, Artificial Sequence
  A polynucleotide encoding a part of a polypeptide of the present invention (number 13 of 22 synthetic gene fragments).
gctttcctgtctgtcaaactgcgtatgaaggagcacaagaagcactcttctggttgtgct

SEQ ID NO: 34 - DNA, 60 bp, Artificial Sequence
  A polynucleotide encoding a part of a polypeptide of the present invention (number 14 of 22 synthetic gene fragments).
ccagagtcagttcttcgaactgcttcttgacagacaggaaagcacaaccagaagagtgct

SEQ ID NO: 35 - DNA, 60 bp, Artificial Sequence
  A polynucleotide encoding a part of a polypeptide of the present invention (number 15 of 22 synthetic gene fragments).
gttcgaagaactgactctgggtgagttcctgaagctgcgtatgaagcagttcgaggagct

SEQ ID NO: 36 - DNA, 60 bp, Artificial Sequence
  A polynucleotide encoding a part of a polypeptide of the present invention (number 16 of 22 synthetic gene fragments).
agcacgttcacgatccagcttcaggaactcacccagagtcagctcctcgaactgcttcat

SEQ ID NO: 37 - DNA, 60 bp, Artificial Sequence
  A polynucleotide encoding a part of a polypeptide of the present invention (number 17 of 22 synthetic gene fragments).
agctggatcgtgaacgtgctaagaacaagatcgctaaggagactaacaacaagctgcgta

SEQ ID NO: 38 - DNA, 60 bp, Artificial Sequence
  A polynucleotide encoding a part of a polypeptide of the present invention (number 18 of 22 synthetic gene fragments).
gtctccttacgatcttgttcttagcacgctcacgcttcattacgcagcttgttgttagtc

SEQ ID NO: 39 - DNA, 60 bp, Artificial Sequence
  A polynucleotide encoding a part of a polypeptide of the invention (number 19 of 22 synthetic gene fragments).
aacaagatcgctaaggagactaacaacaagaagaaggagttcgaggagactgctgagaag

SEQ ID NO: 40 - DNA, 60 bp, Artificial Sequence
  A polynucleotide encoding a part of a polypeptide of the present invention (number 20 of 22 synthetic gene fragments).
cctcgaactccttcttcatacgaaggatagcacgacgaaccttctcagcagtctcctcga

SEQ ID NO: 41 - DNA, 60 bp, Artificial Sequence
  A polynucleotide encoding a part of a polypeptide of the invention(number 21 of 22 synthetic gene fragments).
tatgaagaaggagttcgaggagactgctgagaaggttcgtcgtgctatcgagcagctggc

SEQ ID NO: 42 - DNA, 39 bp, Artificial Sequence
  A polynucleotide encoding a part of a polypeptide of the invention (number 22 of 22 synthetic gene fragments).
ctcgagttagtccatggcagccagctgctcgatagcacg

SEQ ID NO: 43 - protein, 98 aa, Human papillomavirus type 16
MHGDTPTLHEYMLDLQPETTDLYCYEQLNDSSEEEDEIDGPAGQAEPDRAHYNIVTFCCKCDSTLRLCVQSTHVDIRTLEDLLMGTLGIVCPICSQKP

SEQ ID NO: 44 - protein, 105 aa, Human papillomavirus type 18
MHGPKATLQDIVLHLEPQNEIPVDLLCHEQLSDSEEENDEIDGVNHQHLPARRAEPQRHTMLCMCCKCEARIKLVVESSADDLRAFQQLFLNTLSFVCPWCASQQ

SEQ ID NO: 45 - protein, 35 aa, Human papillomavirus type 16
MHGDTPTLHEYMLDLQPETTDLYCYEQLNDSSEEE

SEQ ID NO: 46 - protein, 35 aa, Human papillomavirus type 16
EQLNDSSEEEDEIDGPAGQAEPDRAHYNIVTFCCK

SEQ ID NO: 47 - protein, 35 aa, Human papillomavirus type 16
HYNIVTFCCKCDSTLRLCVQSTHVDIRTLEDLLMG

SEQ ID NO: 48 - protein, 23 aa, Human papillomavirus type 16
IRTLEDLLMGTLGIVCPICSQKP

SEQ ID NO: 49 - protein, 140 aa, Artificial Sequence
  A polypeptide comprising peptide fragments derived from HPV16 E7.
MHGDTPTLHEYMLDLQPETTDLYCYEQLNDSSEEELRMKEQLNDSSEEEDEIDGPAGQAEPDRAHYNIVTFCCKLRMKHYNIVTFCCKCDSTLRLCVQSTHVDIRTLEDLLMGLRMKIRTLEDLLMGTLGIVCPICSQKP

SEQ ID NO: 50 - DNA, 423 bp, Artificial Sequence
  A polynucleotide encoding a polynucleotide according to the present invention comprising sequences derived from HPV16 E7.
atgcatggtgataccccgaccctgcatgaatatatgctggatctgcaaccggaaaccaccgatctgtattgttatgagcagctgaatgatagcagcgaagaggaattacgcatgaaggaacagctgaacgattcaagcgaagaagaggacgaaattgacggtccggcaggtcaggcagaaccggatcgtgcacattacaacattgttaccttttgttgcaaactgagaatgaaacactacaatatcgtgaccttctgctgtaaatgtgatagcaccctgcgtctgtgtgttcagagcacccatgttgatattcgtacattagaggacctgctgatgggcctgcggatgaaaattcgtaccctggaagacctgttaatgggcaccctgggtattgtttgtccgatttgtagccagaaaccgtaa

SEQ ID NO: 51 - protein, 279 aa, Artificial Sequence
  A polypeptide accroding to the present invention comprising sequences derived from Mus musculus survivin.
MHHHHHHGAPALPQIWQLYLKNYRIATFKNWPFLEDLRMKNYRIATFKNWPFLEDCACTPERMAEAGFIHLRMKCACTPERMAEAGFIHCPTENEPDLAQCFFCLRMKCPTENEPDLAQCFFCFKELEGWEPDDNPIELRMKFKELEGWEPDDNPIEEHRKHSPGCAFLTVKLRMKEHRKHSPGCAFLTVKKQMEELTVSEFLKLDLRMKKQMEELTVSEFLKLDRQRAKNKIAKETNNKLRMKRQRAKNKIAKETNNKQKEFEETAKTTRQSIEQLAA

SEQ ID NO: 52 - DNA, 294 bp, Human papillomavirus type 16
atgcacggcgacacccccaccctgcacgagtacatgctggacctgcagcccgagaccaccgacctgtactgctacgagcagctgaacgacagcagcgaggaggaggacgagatcgacggccccgccggccaggccgagcccgacagggcccactacaacatcgtgaccttctgctgcaagtgcgacagcaccctgaggctgtgcgtgcagagcacccacgtggacatcaggaccctggaggacctgctgatgggcaccctgggcatcgtgtgccccatctgcagccagaagccc